(19) United States
(12) Patent Application Publication
(10) Pub. No.: US 2024/0228502 A1
(43) Pub. Date: Jul. 11, 2024

(54) 2-PHENYLAMINO PYRROLOPYRIMIDINES AS ACK1 INHIBITORS

(71) Applicant: H. LEE MOFFITT CANCER CENTER AND RESEARCH INSTITUTE, INC., Tampa, FL (US)

(72) Inventors: Nicholas LAWRENCE, Tampa, FL (US); Harshani LAWRENCE, Tampa, FL (US); Derek DUCKETT, Land O Lakes, FL (US); Gary REUTHER, Temple Terrace, FL (US)

(21) Appl. No.: 18/556,512

(22) PCT Filed: Apr. 22, 2022
(86) PCT No.: PCT/US2022/025919

§ 371 Date: Oct. 20, 2023

Related U.S. Application Data
(60) Provisional application No. 63/178,350, filed on Apr. 22, 2021.

(51) Int. Cl. C07D 487/04; A61K 31/519; A61K 31/541

(52) U.S. Cl. CPC C07D 487/04; A61K 31/519; A61K 31/541

(57) ABSTRACT

This disclosure provides compounds useful for treating medical disorder, and more particularly ACK1 inhibitors useful for treating cancers.

### CROSS-REFERENCE TO RELATED APPLICATIONS

This application claims the benefit of priority to U.S. Provisional Application No. 63/178,350, filed Apr. 22, 2021.

### STATEMENT REGARDING FEDERALLY SPONSORED RESEARCH OR DEVELOPMENT

This invention was made with government support under Grant No. R50-CA211447 awarded by the National Institutes of Health and the National Cancer Institute. The Government has certain rights in the invention.

### TECHNICAL FIELD

This disclosure relates to compounds useful for treating medical disorder, and more particularly to ACK1 inhibitors useful for treating cancers.

### BACKGROUND

ACK1 (activated CDC42 kinase 1), also known as TNK2, is an enzyme that is encoded by the TNK2 gene in humans. ACK1, a non-receptor tyrosine kinase, is activated or mutated in various types of cancers such as prostate, breast, ovarian, leukemia (MPN) and lung cancers. ACK1 also interacts with tyrosine-kinase B (AKT), androgen receptor (AR), a tumor suppressor oxidoreductase (W_{wox}), protooncogene tyrosine-protein kinase (FYN), and growth factor receptor protein 2 (Grb) by phosphorylating specific tyrosine residues (see Manser E, Leung T, Salihuddin H, Tan L, Lim L. A non-receptor tyrosine kinase that inhibits the GTPase activity of p21. Nature, 363, 364-367(1993); Mahajan K, Mahajan N, Shepherding AKT and Androgen Receptor by Ack1 Tyrosine Kinase, J. Cell. Physiol. 224: 327-333, 2010; Yokoyama N, Millerv W T. Biochemical Properties of the Cdc42-associated Tyrosine Kinase ACK1. J. Biol. Chem. 2003, 278, 47713-47723; and Mahajan K, Coppola D, Challa S, Fang B, Chen Y A, et al. (2010). Ack1 Mediated AKT/PKB Tyrosine 176 Phosphorylation Regulates Its Activation. PLoSONE 5(3): e9646. doi:10.1371/journal.pone.0009646).

Mechanistically, ACK1 phosphorylates Tyr176 of AKT and activated PI3K-independent AKT, thus facilitating the survival of breast cells by suppressing proapoptotic pathways. As a result, breast cancer is progressed. Contrarily, knockdown of ACK1 expression by siRNA caused suppression of AKT Tyr176 phosphorylation and increased the expression of proapoptotic genes such as Bim and FAS. The importance of ACK1 activation in tumor initiation and progression garners investigation of ACK1 inhibition as an attractive target for the development of anticancer drugs (see XianYun Jiao et sl. Synthesis and optimization of substituted furo[2,3-d]-pyrimidin-4-amines and 7H-pyrrolo[2,3-d]pyrimidin-4-amines as ACK1 inhibitors. Bioorg. Med. Chem. Lett. 22 (2012) 6212-6217; and Lawrence H, Mahajan K, Luo Y, Zhang D, Tindall N, Huseyin M, Gevariya H, Kazi S, Ozcan S, Mahajan N, Lawrence N, Development of Novel ACK1/TNK2 Inhibitors Using a Fragment-Based Approach. 2015, 58(6), 2746-63).

There is clearly a need for development of inhibitors of ACK1 which may be useful in the treatment of cancers. This disclosure addresses this as well as other needs.

### SUMMARY

The present disclosure provides compounds and compositions which are useful in the treatment of medical disorders, such as cancers, and methods of use for the same. In particular, this disclosure provides 2-phenylamino pyrrolopyrimidines which are ACK1 inhibitors having use in the treatment of cancers.

In one aspect, a compound is provided of Formula I

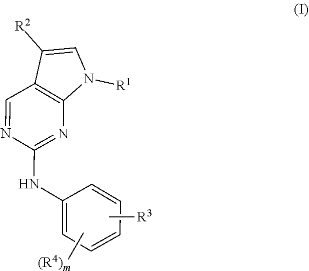

or a pharmaceutically acceptable salt thereof,
wherein all variables are as defined herein.

A pharmaceutical composition is also provided comprising a compound described herein, or a pharmaceutically acceptable salt thereof, and a pharmaceutically acceptable carrier or excipient.

A method of treating a cancer in a subject in need thereof is also provided, the method comprising administering to the subject a therapeutically effective amount of a compound described herein, or a pharmaceutically acceptable salt thereof, or a pharmaceutical composition as described herein.

A method of killing a tumor cell comprising contacting the tumor cell with an effective amount of a compound of any one of claims 1—described herein, or a pharmaceutically acceptable salt thereof, or a composition as described herein.

The details of one or more embodiments of the disclosure are set forth in the accompanying drawings and the description below. Other features, objects, and advantages of the disclosure will be apparent from the description and drawings, and from the claims.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

The present disclosure 2-phenylaminopyrrolopyrimidine compounds which are inhibitors of activated CDC42 kinase 1 (ACK1) and are useful in the treatment of cancers.

In one aspect, a compound is provided of Formula I

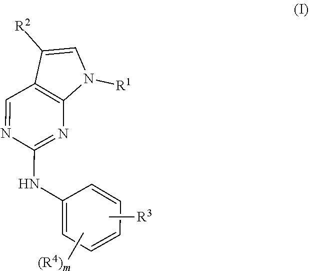

or a pharmaceutically acceptable salt thereof,
wherein:
R¹ is selected from —(C₀-C₃ alkyl)(3- to 8-membered monocyclic or bicyclic heterocyclyl) and —(C₀-C₃ alkyl)(6- to 10-membered monocyclic or bicyclic aryl), wherein R¹ may be optionally substituted with one or more (for example, 1, 2, 3, or 4) groups selected from Z as allowed by valency;
R² is selected from hydrogen, halogen, and C₁-C₆ alkyl;
R³ is selected from —(C₀-C₃ alkyl)(3- to 8-membered monocyclic or bicyclic heterocyclyl) and —(C₀-C₃ alkyl)(5- to 10-membered moncyclic or bicyclic heteroaryl), wherein R³ may be optionally substituted with one or more (for example, 1, 2, 3, or 4) groups selected from Z as allowed by valency;
m is 0, 1, 2, 3, or 4;
R⁴ is independently selected at each occurrence from hydrogen, halogen, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted one or more groups selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some embodiments of Formula I, R¹ is —(C₀-C₃ alkyl)(3- to 8-membered monocyclic or bicyclic heterocyclyl) optionally substituted with one or more Z groups. In some embodiments of Formula I, R¹ is —(C₀-C₃ alkyl)(5- to 6-membered monocyclic or bicyclic heterocyclyl) optionally substituted with one or more Z groups. In some embodiments of Formula I, R¹ is —(C₀-C₃ alkyl)(tetrahydrofuranyl or tetrohydropyranyl) optionally substituted with one or more Z groups. In some embodiments of Formula I, R¹ is selected from —CH₂(tetrahydrofuranyl) or —CH₂(tetrahydropyranyl) optionally substituted with one or more Z groups.

In some embodiments, R¹ is selected from:

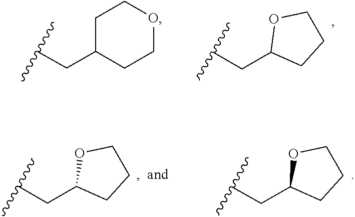 [SMILES: *CC1CCOCC1 ; *CC1CCCO1 ; *C[C@H]1CCCO1 ; *C[C@@H]1CCCO1]

In some embodiments of Formula I, R¹ is —(C₀-C₃ alkyl)(6- to 10-membered monocyclic or bicyclic aryl) optionally substituted with one or more Z groups. In some embodiments of Formula I, R¹ is —(C₀-C₃ alkyl)(phenyl) optionally substituted with one or more Z groups.

In some embodiments, R¹ is selected from:

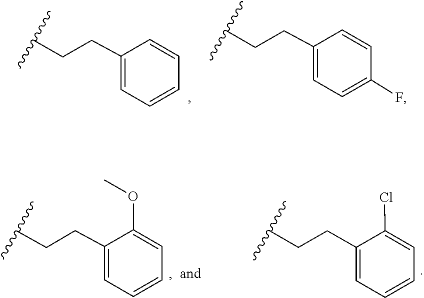 [SMILES: *CCc1ccccc1 ; *CCc1ccc(F)cc1 ; *CCc1ccccc1OC ; *CCc1ccccc1Cl]

In some embodiments, R¹ is phenyl optionally substituted with one or more Z groups. In some embodiments, R¹ is phenyl substituted with a group selected from —NHS(O)₂(C₁-C₆ alkyl) and

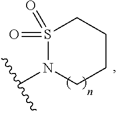

wherein n is 0 or 1.

In some embodiments R¹ is selected from:

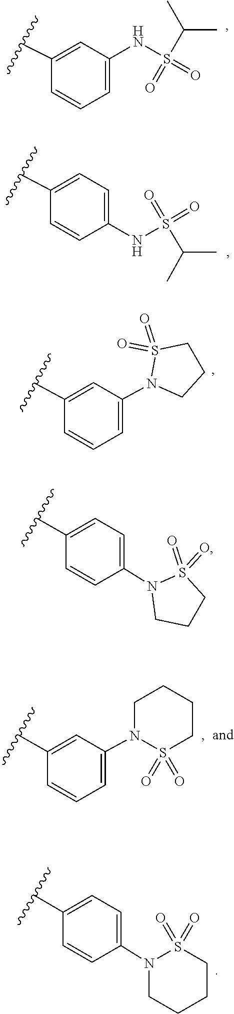

In some embodiments of Formula I, R² is hydrogen. In some embodiments of Formula I, R² is halogen. In some embodiments of Formula I, R² is fluoro. In some embodiments of Formula I, R² is C₁-C₆ alkyl. In some embodiments of Formula I, R² is methyl.

In some embodiments of Formula I,

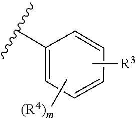

is selected from:

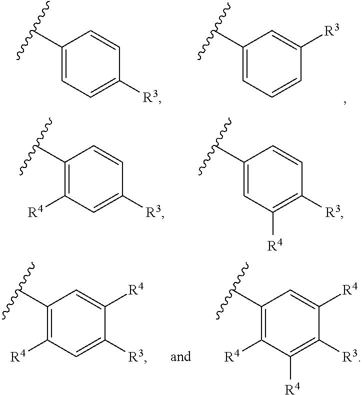

In some embodiments of Formula I,

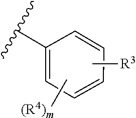

is selected from:

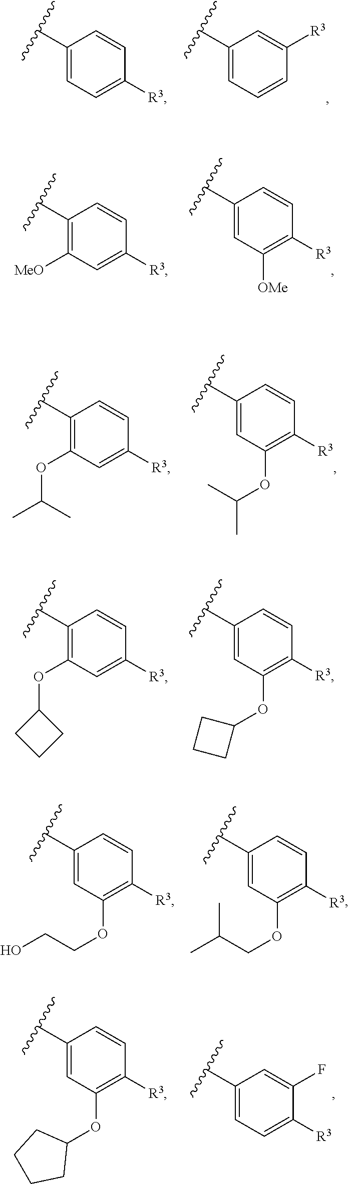

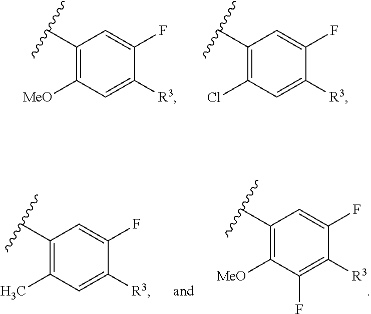

In some embodiments of Formula I,

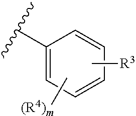

is selected from:

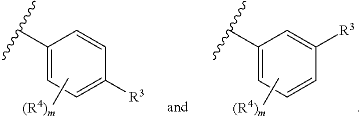

In some embodiments of Formula I, R³ is —(C₀-C₃ alkyl)(3- to 8-membered monocyclic or bicyclic heterocyclyl) optionally substituted with one or more Z groups. In some embodiments of Formula I, R³ is —(C₀-C₃ alkyl)(5- to 6-membered monocyclic heterocyclyl) optionally substituted with one or more Z groups. In some embodiments of Formula I, R³ is 5- to 6-membered monocyclic heterocyclyl optionally substituted with one or more Z groups. In some embodiments of Formula I, R³ is selected from piperidinyl or piperazinyl, each of which may be optionally substituted with one or more Z groups.

In some embodiments of Formula I, R³ is —(C₀-C₃ alkyl)(5- to 10-membered monocyclic or bicyclic heteroaryl) optionally substituted with one or more Z groups. In some embodiments of Formula I, R³ is —(C₀-C₃ alkyl)(5- to 6-membered monocyclic heteroaryl) optionally substituted with one or more Z groups. In some embodiments of Formula I, R³ is 5- to 6-membered monocyclic heteroaryl optionally substituted with one or more Z groups. In some embodiments of Formula I, R³ is pyrrolyl or pyrazolyl optionally substituted with one or more Z groups.

In some embodiments of Formula I, R³ is selected from:

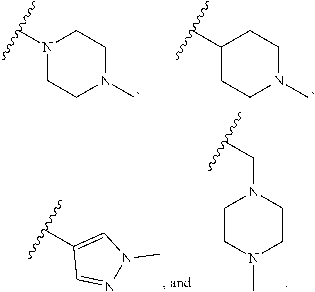

In some embodiments of Formula I,

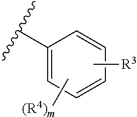

is selected from:

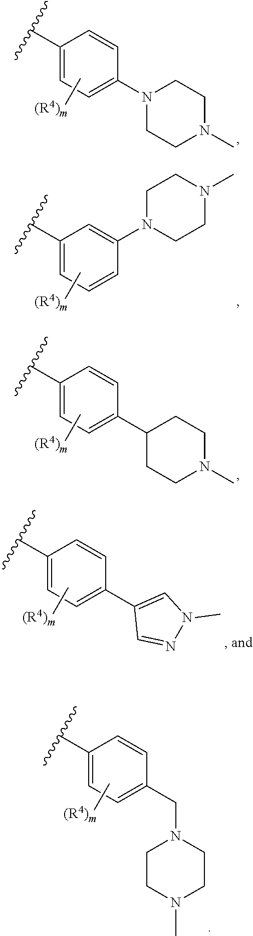

In some embodiments of Formula I,

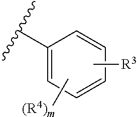

is selected from:

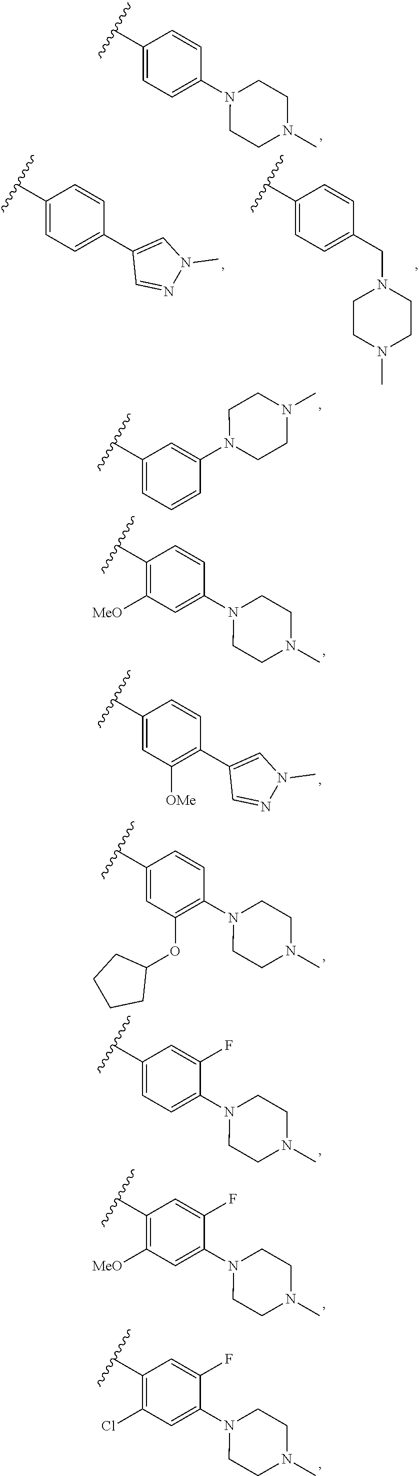

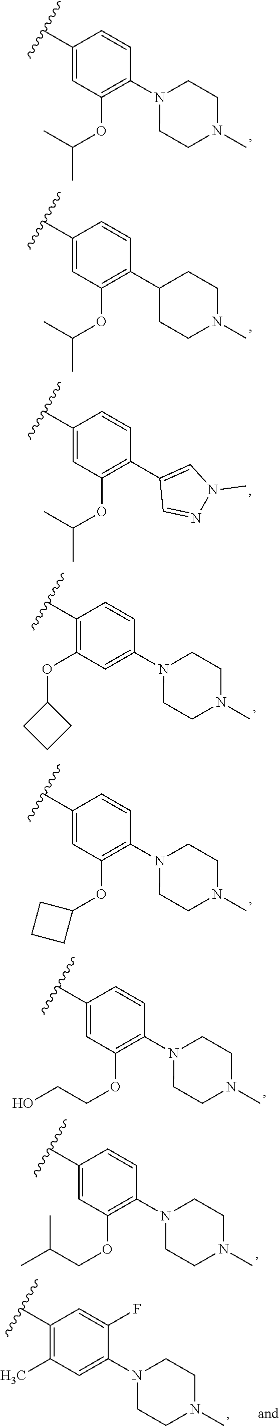

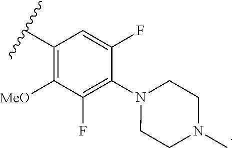 [SMILES: *c1cc(F)c(N2CCN(C)CC2)c(F)c1OC]

Representative examples of compounds of Formula I include:

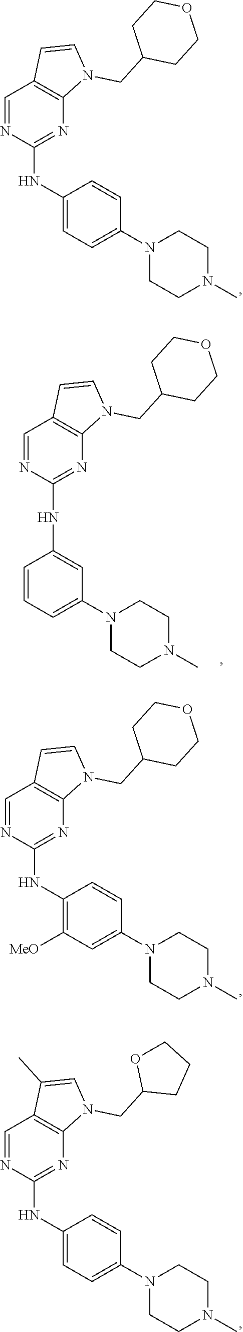

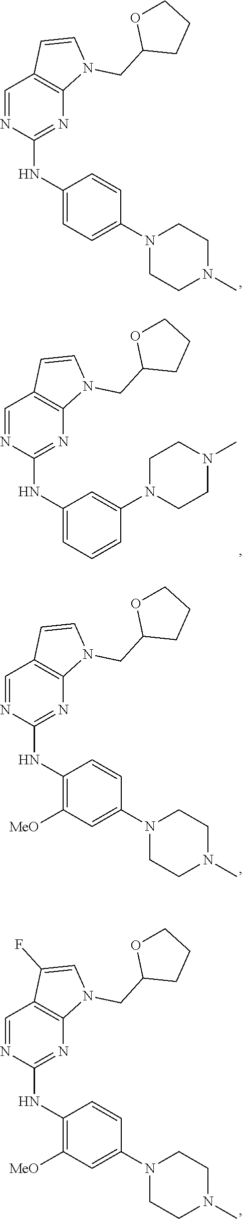

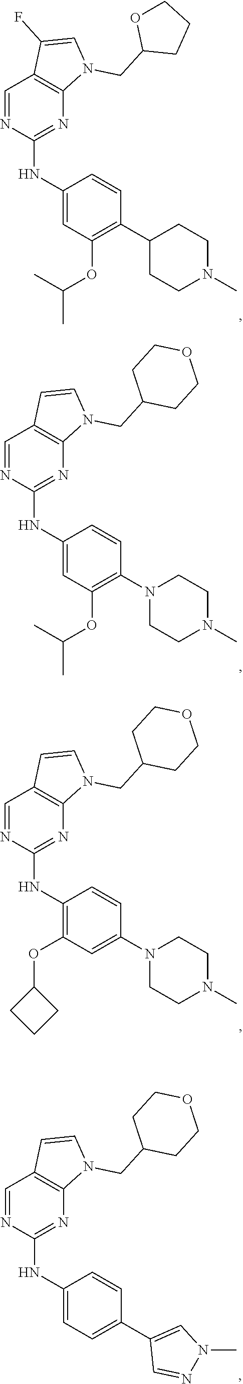 [SMILES: CC(C)Oc1cc(Nc2ncc3c(F)cn(CC4CCCO4)c3n2)ccc1C1CCN(C)CC1 ; CC(C)Oc1cc(Nc2ncc3ccn(CC4CCOCC4)c3n2)ccc1N1CCN(C)CC1 ; CN1CCN(c2ccc(Nc3ncc4ccn(CC5CCOCC5)c4n3)c(OC3CCC3)c2)CC1 ; Cn1cc(-c2ccc(Nc3ncc4ccn(CC5CCOCC5)c4n3)cc2)cn1]

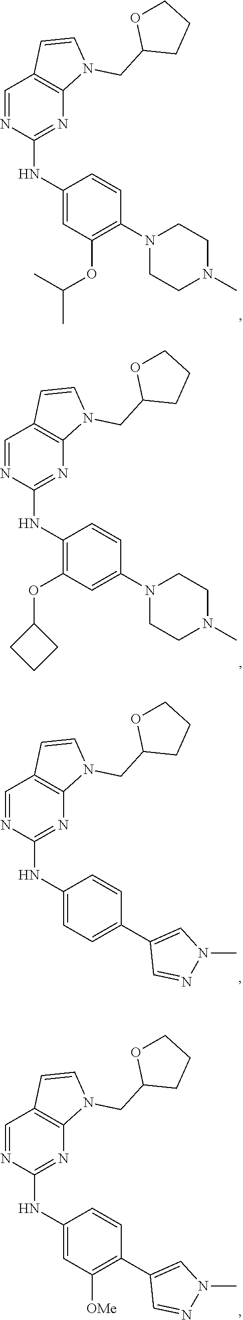 [SMILES: CC(C)Oc1cc(Nc2ncc3ccn(CC4CCCO4)c3n2)ccc1N1CCN(C)CC1 ; CN1CCN(c2ccc(Nc3ncc4ccn(CC5CCCO5)c4n3)c(OC3CCC3)c2)CC1 ; Cn1cc(-c2ccc(Nc3ncc4ccn(CC5CCCO5)c4n3)cc2)cn1 ; COc1cc(Nc2ncc3ccn(CC4CCCO4)c3n2)ccc1-c1cnn(C)c1]

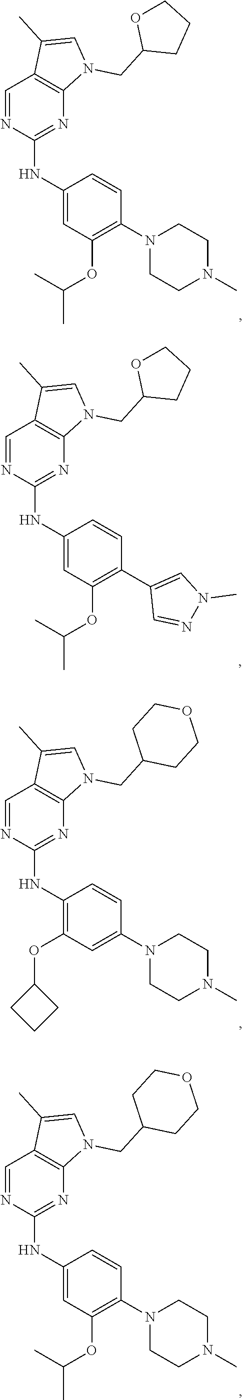 [SMILES: Cc1cn(CC2CCCO2)c2nc(Nc3ccc(N4CCN(C)CC4)c(OC(C)C)c3)ncc12 ; Cc1cn(CC2CCCO2)c2nc(Nc3ccc(-c4cnn(C)c4)c(OC(C)C)c3)ncc12 ; Cc1cn(CC2CCOCC2)c2nc(Nc3ccc(N4CCN(C)CC4)cc3OC3CCC3)ncc12 ; Cc1cn(CC2CCOCC2)c2nc(Nc3ccc(N4CCN(C)CC4)c(OC(C)C)c3)ncc12]

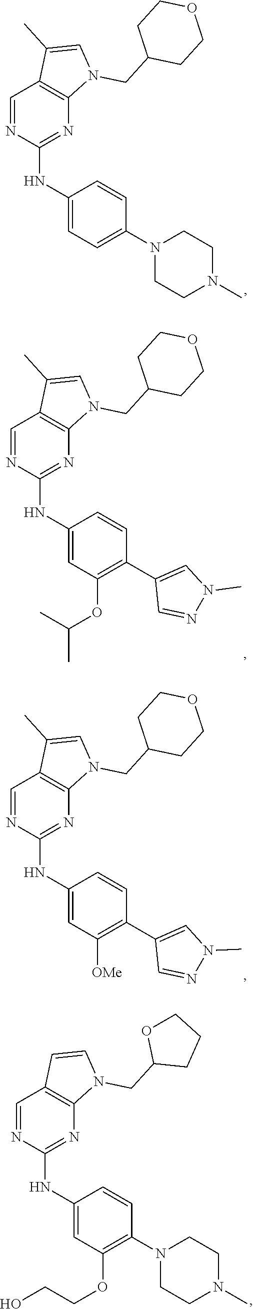

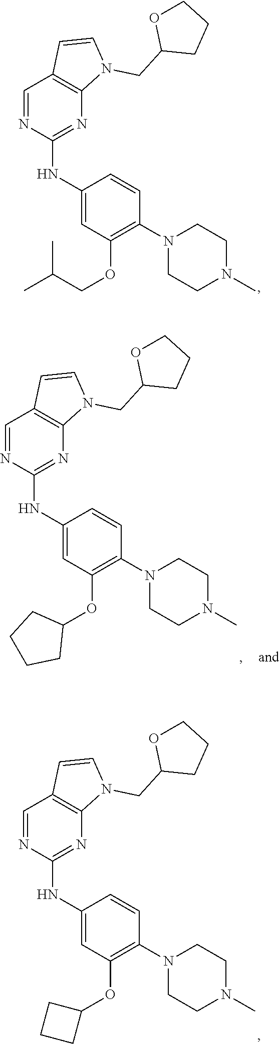 [SMILES: CC(C)COc1cc(Nc2ncc3ccn(CC4CCCO4)c3n2)ccc1N1CCN(C)CC1 ; CN1CCN(c2ccc(Nc3ncc4ccn(CC5CCCO5)c4n3)cc2OC2CCCC2)CC1 ; CN1CCN(c2ccc(Nc3ncc4ccn(CC5CCCO5)c4n3)cc2OC2CCC2)CC1]

or pharmaceutically acceptable salts thereof.

Further representative examples of compounds of Formula I include:

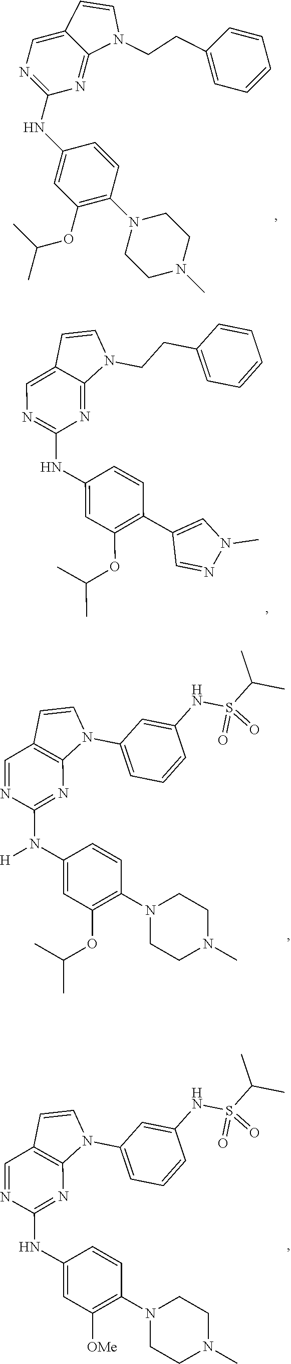

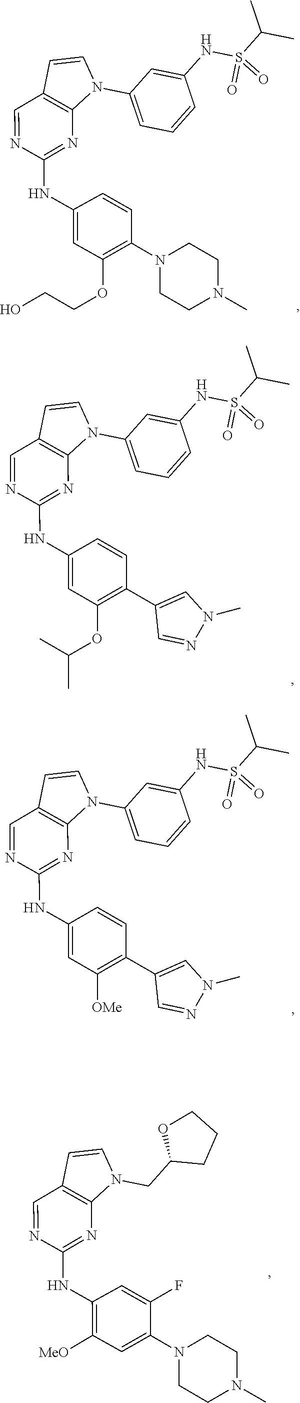

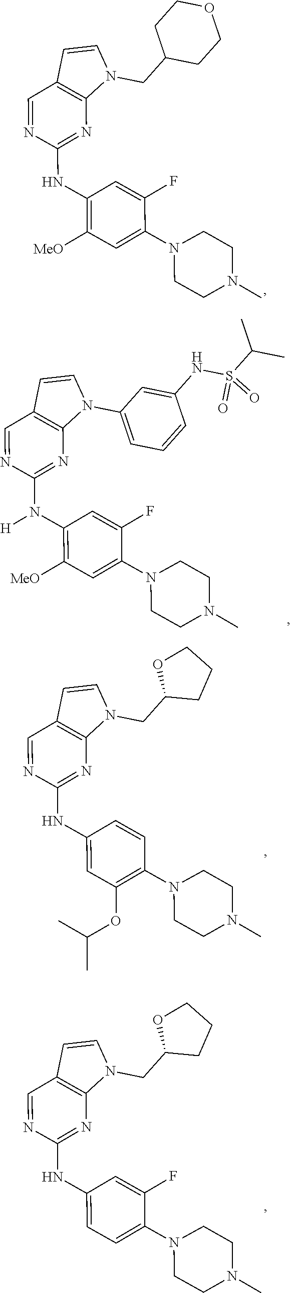

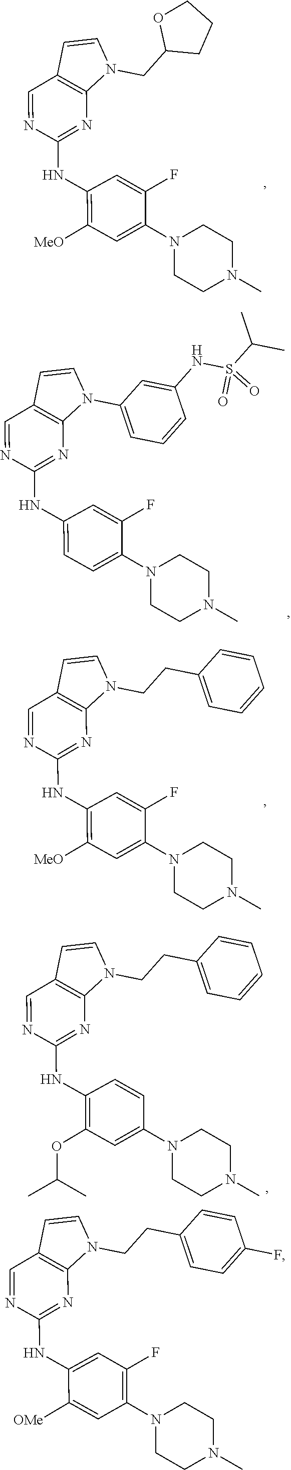

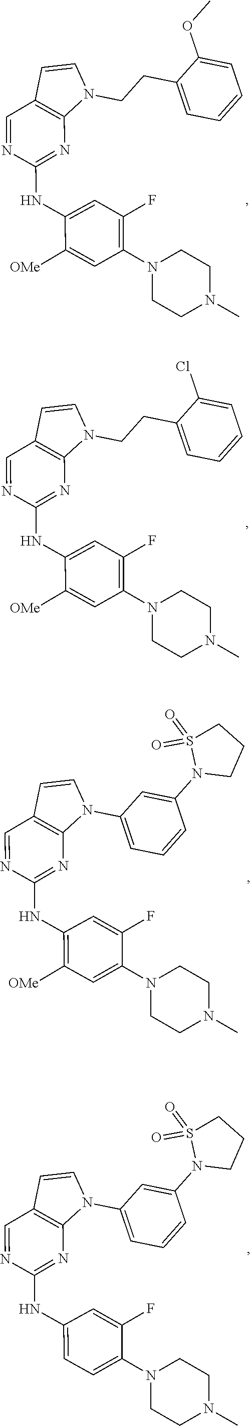 [SMILES: COc1ccccc1CCn1ccc2cnc(Nc3cc(F)c(N4CCN(C)CC4)cc3OC)nc21 ; COc1cc(N2CCN(C)CC2)c(F)cc1Nc1ncc2ccn(CCc3ccccc3Cl)c2n1 ; COc1cc(N2CCN(C)CC2)c(F)cc1Nc1ncc2ccn(-c3cccc(N4CCCS4(=O)=O)c3)c2n1 ; CN1CCN(c2ccc(Nc3ncc4ccn(-c5cccc(N6CCCS6(=O)=O)c5)c4n3)cc2F)CC1]

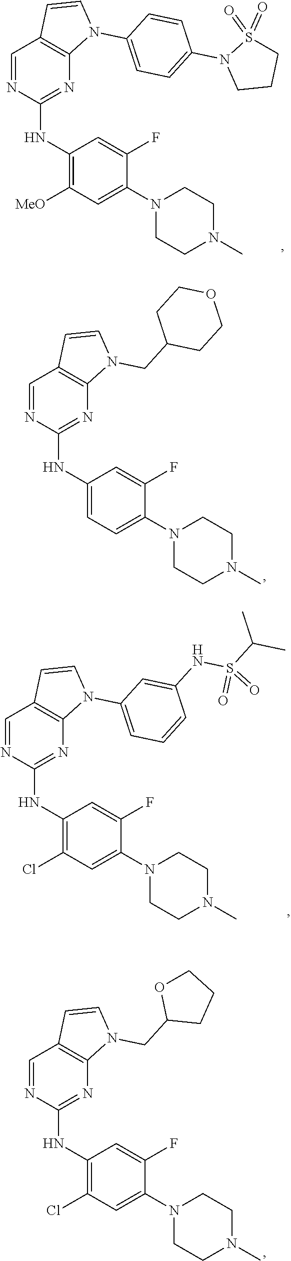

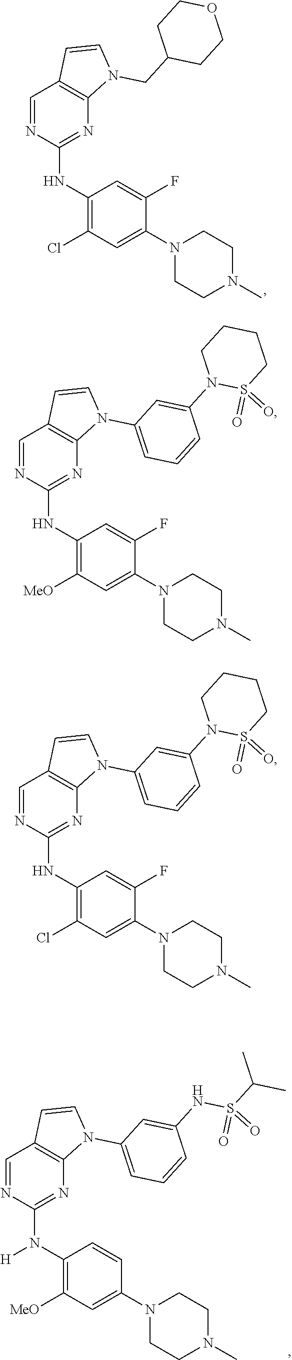

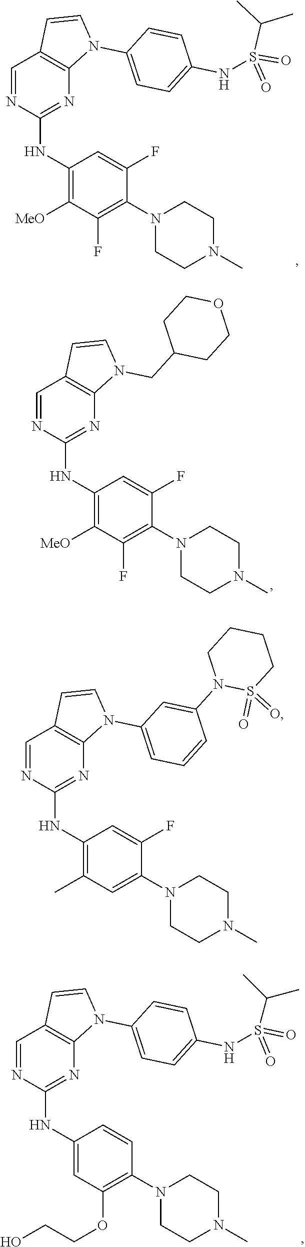

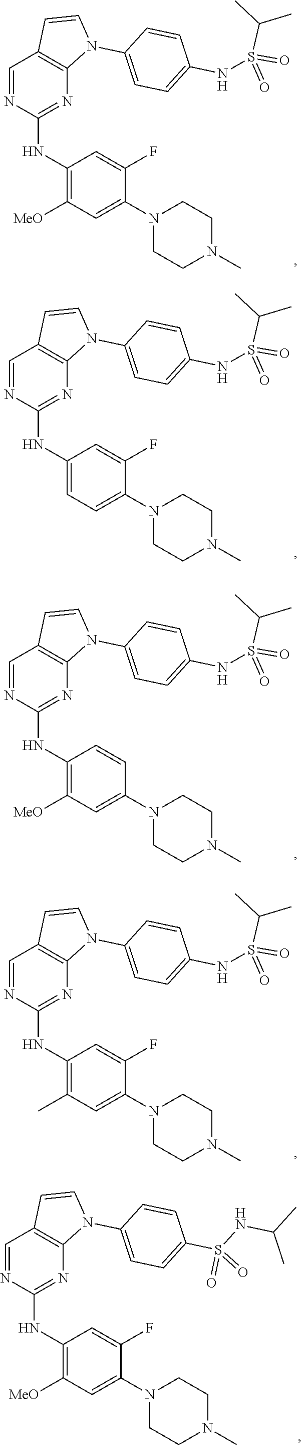

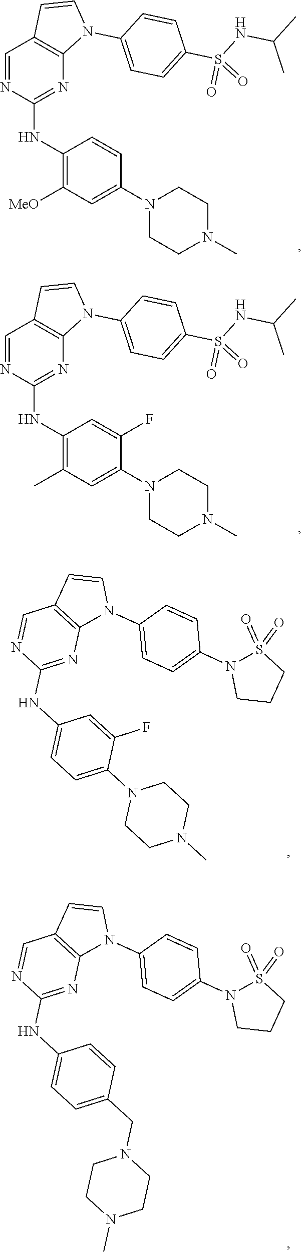 [SMILES: COc1cc(N2CCN(C)CC2)ccc1Nc1ncc2ccn(-c3ccc(S(=O)(=O)NC(C)C)cc3)c2n1 ; Cc1cc(N2CCN(C)CC2)c(F)cc1Nc1ncc2ccn(-c3ccc(S(=O)(=O)NC(C)C)cc3)c2n1 ; CN1CCN(c2ccc(Nc3ncc4ccn(-c5ccc(N6CCCS6(=O)=O)cc5)c4n3)cc2F)CC1 ; CN1CCN(Cc2ccc(Nc3ncc4ccn(-c5ccc(N6CCCS6(=O)=O)cc5)c4n3)cc2)CC1]

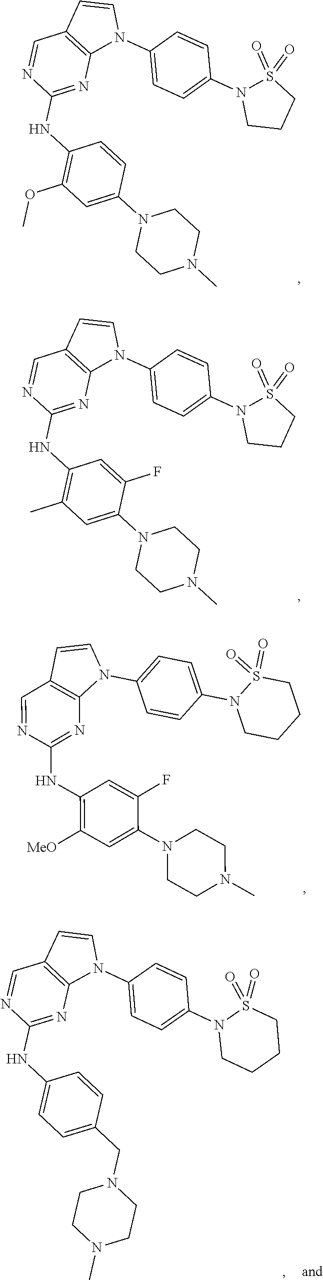 [SMILES: COc1cc(N2CCN(C)CC2)ccc1Nc1ncc2ccn(-c3ccc(N4CCCS4(=O)=O)cc3)c2n1 ; Cc1cc(N2CCN(C)CC2)c(F)cc1Nc1ncc2ccn(-c3ccc(N4CCCS4(=O)=O)cc3)c2n1 ; COc1cc(N2CCN(C)CC2)c(F)cc1Nc1ncc2ccn(-c3ccc(N4CCCCS4(=O)=O)cc3)c2n1 ; CN1CCN(Cc2ccc(Nc3ncc4ccn(-c5ccc(N6CCCCS6(=O)=O)cc5)c4n3)cc2)CC1]

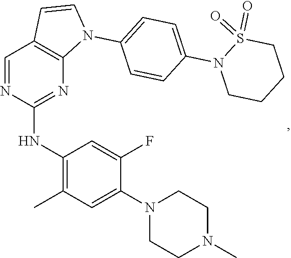 [SMILES: Cc1cc(N2CCN(C)CC2)c(F)cc1Nc1ncc2ccn(-c3ccc(N4CCCCS4(=O)=O)cc3)c2n1]

or pharmaceutically acceptable salts thereof.

Compounds of Formula I may be prepared according to the following representative, albeit non-limiting, procedures:

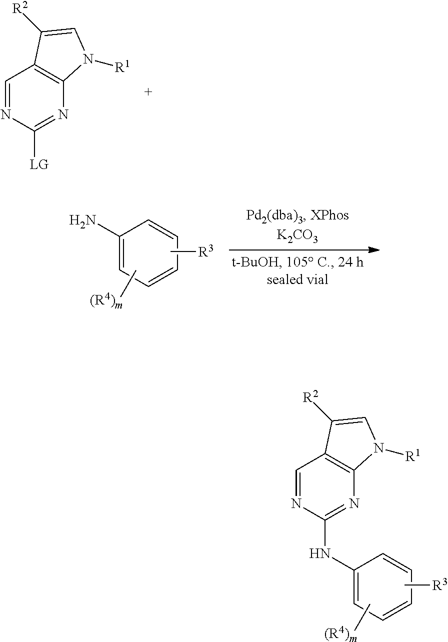

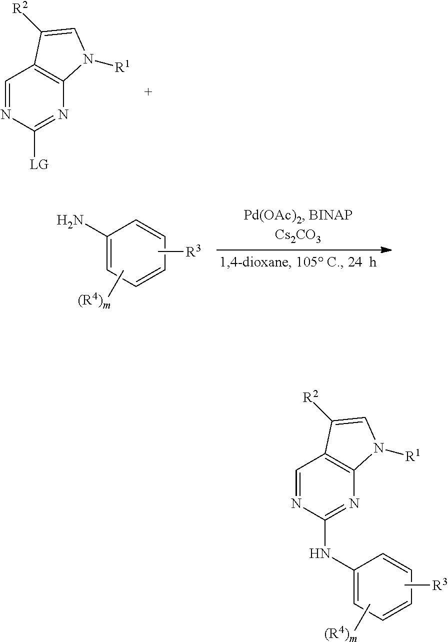

wherein LG is a leaving group. “Leaving group”, as used herein, refers to a molecule or a molecular fragment (e.g., an anion) that is displaced in a chemical reaction as a stable species, taking with it the bonding electrons. Examples of leaving groups include arylsulfonyloxy groups or alkylsulfonyloxy groups, such as mesylate or tosylate. Common anionic leaving groups also include halides such as Cl⁻, Br⁻, and I⁻.

Variations on compounds used in the processes for the preparation of compounds of Formula I can include the addition, subtraction, or movement of various constituents as described for each compounds. Similarly, when one or more chiral centers is present in a molecule, the chirality of the molecule can be changes. Additionally, the synthesis of the compounds used in these processes can involve the protection of various chemical groups, and further the compounds of Formula I prepared by the disclosed processes may be subsequently deprotected as appropriate. The use of protection and deprotection, and the selection of appropriate protecting groups, would be readily known to one skilled in the art. “Protecting group”, as used herein, refers to any convention functional group that allows one to obtain chemoselectivity in a subsequent chemical reaction. Protecting groups are described, for example, in Peter G. M. Wuts, Greene's Protective Groups in Organic Synthesis, 5^{th} Ed., Wiley & Sons, 2014. For a particular compound and/or a particular chemical reaction, a person skilled in the art knows how to select and implement appropriate protecting groups and their associated synthetic methods. Examples of amine protecting groups include acyl and alkoxy carbonyl groups, such a t-butoxycarbonyl (BOC) and [2-(trimethylsilyl)ethoxy]methoxy (SEM). Examples of carboxyl protecting groups include C₁-C₆ alkoxy groups, such as methyl, ethyl, and t-butyl. Examples of alcohol protecting groups include benzyl, trityl, silyl ethers, and the like.

The described processes, or reaction to produce the compounds used in the described processes, can be carried out in solvents indicated herein, or in solvents which can be selected by one of skill in the art of organic synthesis. Solvents can be substantially nonreactive with the starting materials (reactants), intermediates, or products under the conditions at which the reaction is carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H and ¹³C), infrared spectroscopy, spectrophotometry (e.g., UV-visible), or mass spectrometry, or by chromatography such as high performance liquid chromatography (HPLC) or thin layer chromatography (TLC).

The present disclosure also includes compounds of Formula I with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### Methods of Use

The present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some embodiments, the compounds can inhibit ACK1. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

The following embodiments of the present invention are also provided:

Embodiment 1. A compound of Formula I

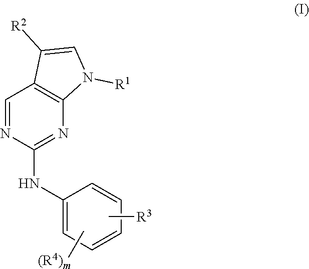

or a pharmaceutically acceptable salt thereof,
wherein:
R¹ is selected from —(C₀-C₃ alkyl)(3- to 8-membered monocyclic or bicyclic heterocyclyl) and —(C₀-C₃ alkyl)(6- to 10-membered monocyclic or bicyclic aryl), wherein R¹ may be optionally substituted with one or more (for example, 1, 2, 3, or 4) groups selected from Z as allowed by valency;
R² is selected from hydrogen, halogen, and C₁-C₆ alkyl;
R³ is selected from —(C₀-C₃ alkyl)(3- to 8-membered monocyclic or bicyclic heterocyclyl) and —(C₀-C₃ alkyl)(5- to 10-membered moncyclic or bicyclic heteroaryl), wherein R³ may be optionally substituted with one or more (for example, 1, 2, 3, or 4) groups selected from Z as allowed by valency;
m is 0, 1, 2, 3, or 4;
R⁴ is independently selected at each occurrence from hydrogen, halogen, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted one or more groups selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.
   Embodiment 2. The compound of embodiment 1, wherein R¹ is —(C₀-C₃ alkyl)(3- to 8-membered monocyclic or bicyclic heterocyclyl) optionally substituted with one or more Z groups.
   Embodiment 3. The compound of embodiment 2, wherein R¹ is —(C₀-C₃ alkyl)(5- to 6-membered monocyclic or bicyclic heterocyclyl) optionally substituted with one or more Z groups.
   Embodiment 4. The compound of embodiment 3, wherein R¹ is —(C₀-C₃ alkyl)(tetrahydrofuranyl or tetrohydropyranyl) optionally substituted with one or more Z groups.
   Embodiment 5. The compound of embodiment 4, R¹ is selected from —CH₂(tetrahydrofuranyl) or —CH₂(tetrahydropyranyl) optionally substituted with one or more Z groups.
   Embodiment 6. The compound of embodiment 5, wherein R¹ is selected from:

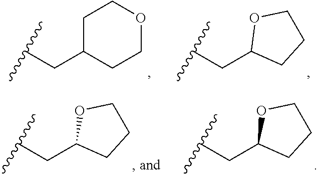

Embodiment 7. The compound of embodiment 1, wherein R¹ is —(C₀-C₃ alkyl)(6- to 10-membered monocyclic or bicyclic aryl) optionally substituted with one or more Z groups.
Embodiment 8. The compound of embodiment 7, wherein R¹ is —(C₀-C₃ alkyl)(phenyl) optionally substituted with one or more Z groups.
Embodiment 9. The compound of embodiment 8, wherein R¹ is selected from:

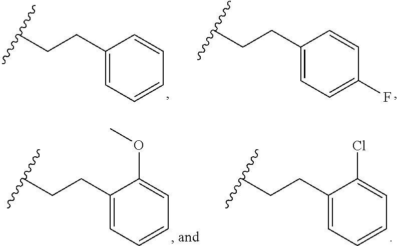

Embodiment 10. The compound of embodiment 1, wherein R¹ is phenyl optionally substituted with one or more Z groups.
Embodiment 11. The compound of embodiment 10, wherein R¹ is phenyl substituted with a group selected from —NHS(O)₂(C₁-C₆ alkyl) and

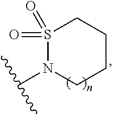

wherein n is 0 or 1.
Embodiment 12. The compound of embodiment 11, wherein R¹ is selected from:

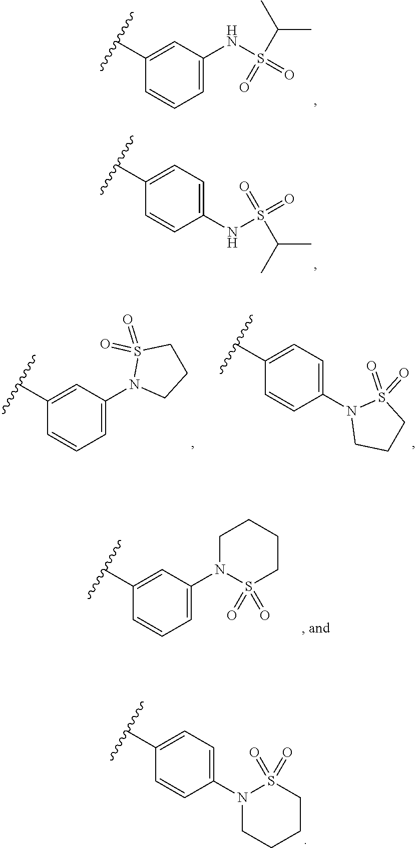 [SMILES: *c1cccc(NS(=O)(=O)C(C)C)c1 ; *c1ccc(NS(=O)(=O)C(C)C)cc1 ; *c1cccc(N2CCCS2(=O)=O)c1 ; *c1ccc(N2CCCS2(=O)=O)cc1 ; *c1cccc(N2CCCCS2(=O)=O)c1 ; *c1ccc(N2CCCCS2(=O)=O)cc1]

Embodiment 13. The compound of any one of embodiments 1-12, wherein R² is hydrogen.
Embodiment 14. The compound of any one of embodiments 1-12, wherein R² is fluoro.
Embodiment 15. The compound of any one of embodiments 1-12, wherein R² is methyl.
Embodiment 16. The compound of any one of embodiments 1-15, wherein

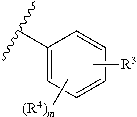

is selected from:

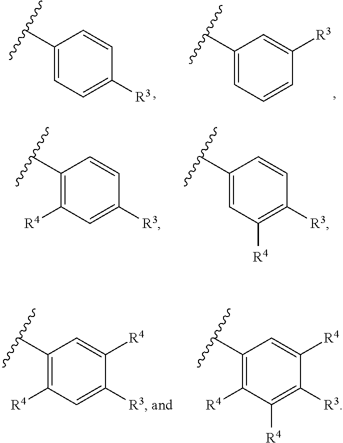

Embodiment 17. The compound of any one of embodiments 1-15, wherein

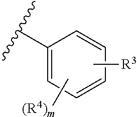

is selected from:

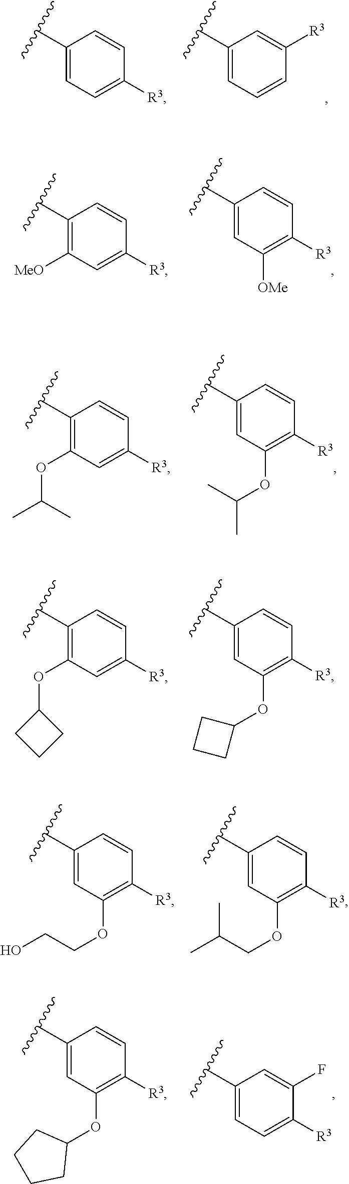

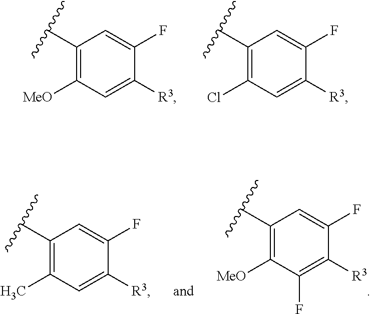

Embodiment 18. The compound of any one of embodiments 1-17, wherein R³ is selected from:

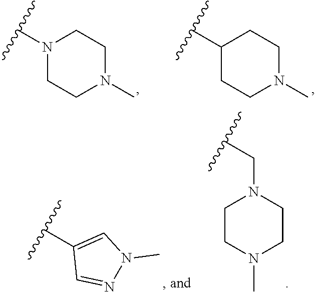

Embodiment 19. The compound of any one of embodiments 1-15, wherein

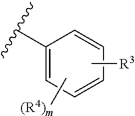

is selected from:

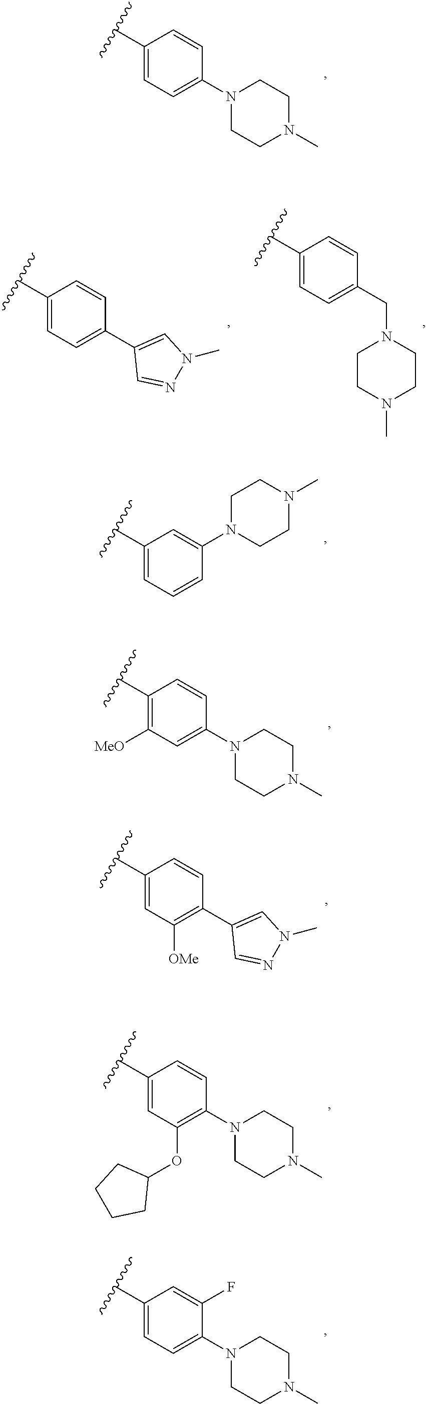

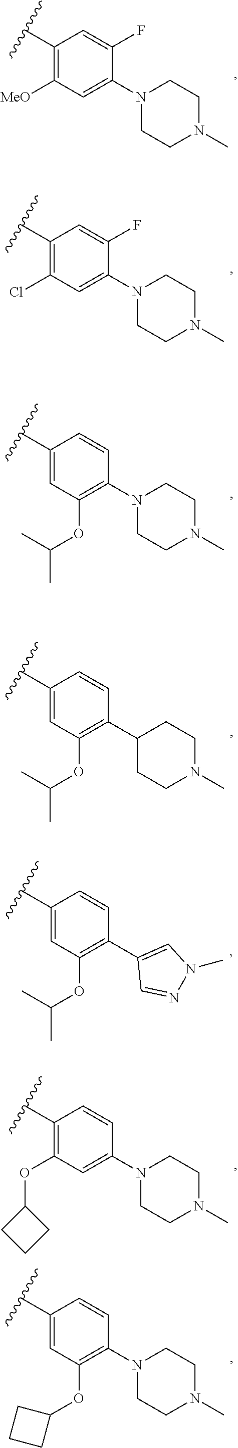

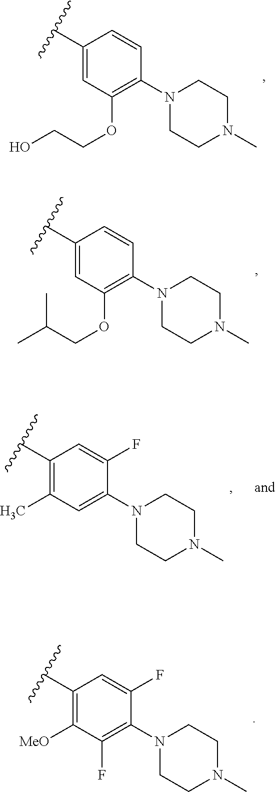

Embodiment 20. A compound selected from:

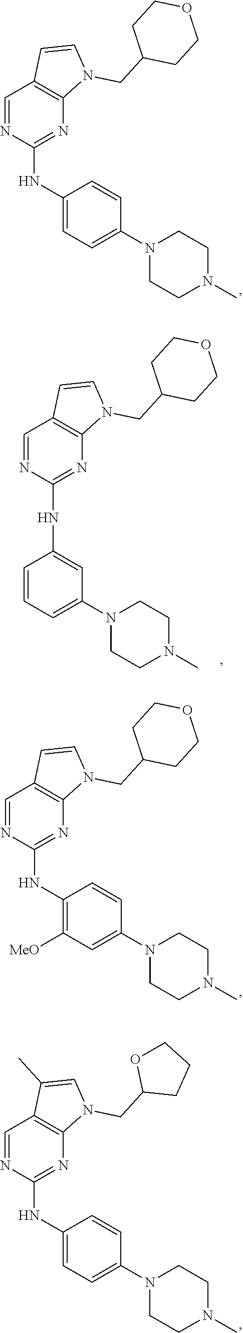

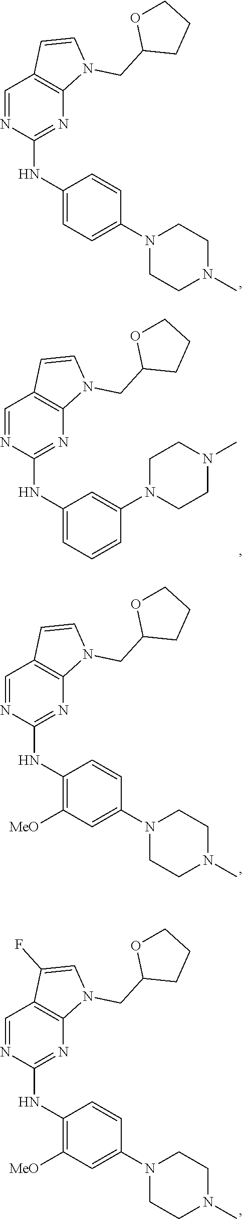

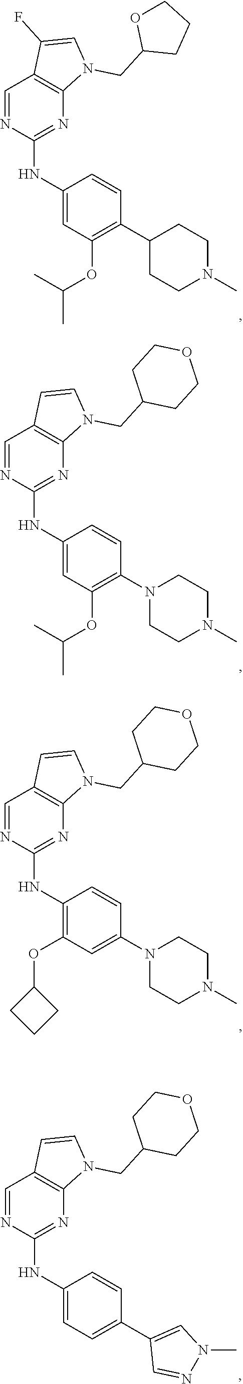 [SMILES: CC(C)Oc1cc(Nc2ncc3c(F)cn(CC4CCCO4)c3n2)ccc1C1CCN(C)CC1 ; CC(C)Oc1cc(Nc2ncc3ccn(CC4CCOCC4)c3n2)ccc1N1CCN(C)CC1 ; CN1CCN(c2ccc(Nc3ncc4ccn(CC5CCOCC5)c4n3)c(OC3CCC3)c2)CC1 ; Cn1cc(-c2ccc(Nc3ncc4ccn(CC5CCOCC5)c4n3)cc2)cn1]

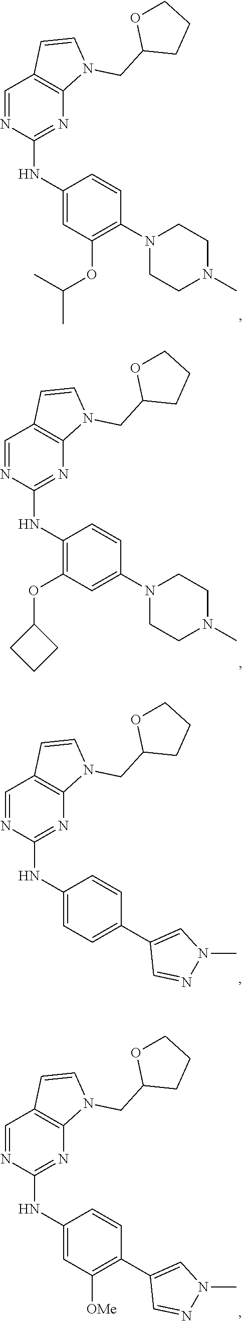 [SMILES: CC(C)Oc1cc(Nc2ncc3ccn(CC4CCCO4)c3n2)ccc1N1CCN(C)CC1 ; CN1CCN(c2ccc(Nc3ncc4ccn(CC5CCCO5)c4n3)c(OC3CCC3)c2)CC1 ; Cn1cc(-c2ccc(Nc3ncc4ccn(CC5CCCO5)c4n3)cc2)cn1 ; COc1cc(Nc2ncc3ccn(CC4CCCO4)c3n2)ccc1-c1cnn(C)c1]

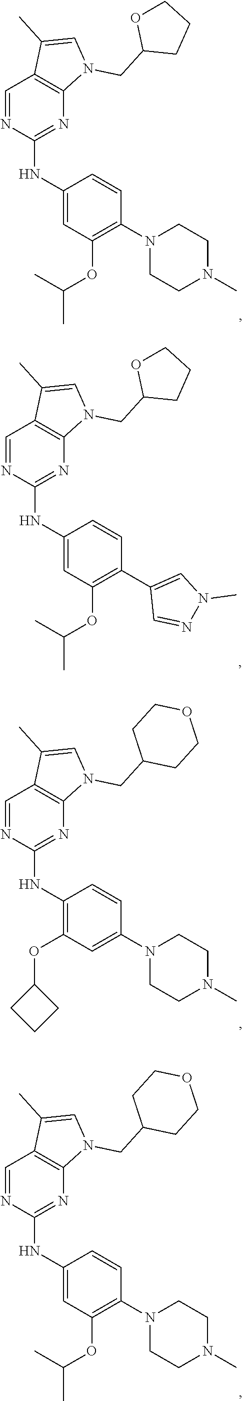 [SMILES: Cc1cn(CC2CCCO2)c2nc(Nc3ccc(N4CCN(C)CC4)c(OC(C)C)c3)ncc12 ; Cc1cn(CC2CCCO2)c2nc(Nc3ccc(-c4cnn(C)c4)c(OC(C)C)c3)ncc12 ; Cc1cn(CC2CCOCC2)c2nc(Nc3ccc(N4CCN(C)CC4)cc3OC3CCC3)ncc12 ; Cc1cn(CC2CCOCC2)c2nc(Nc3ccc(N4CCN(C)CC4)c(OC(C)C)c3)ncc12]

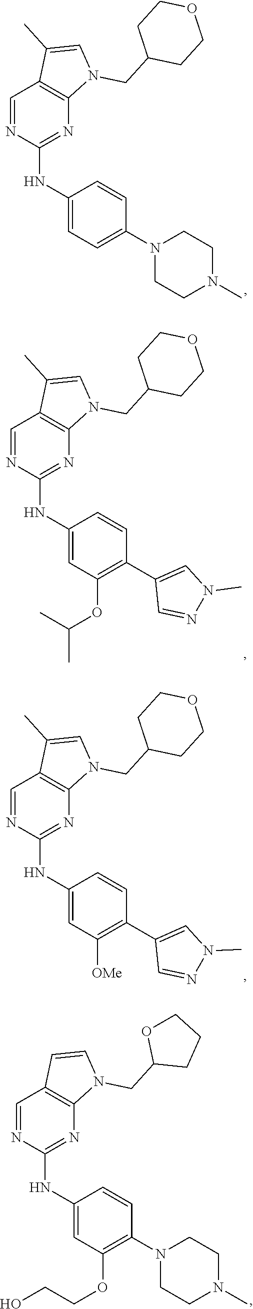

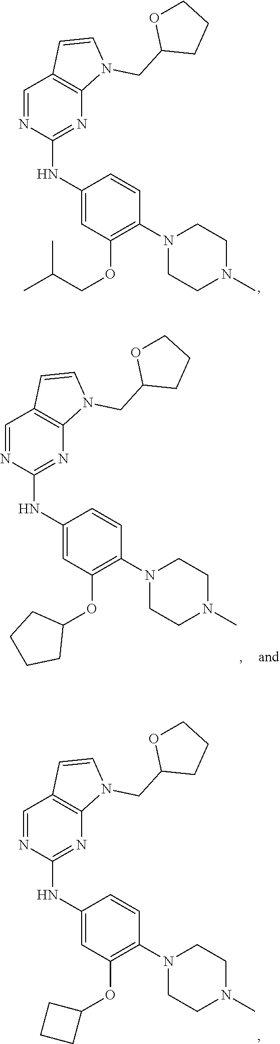 [SMILES: CC(C)COc1cc(Nc2ncc3ccn(CC4CCCO4)c3n2)ccc1N1CCN(C)CC1 ; CN1CCN(c2ccc(Nc3ncc4ccn(CC5CCCO5)c4n3)cc2OC2CCCC2)CC1 ; CN1CCN(c2ccc(Nc3ncc4ccn(CC5CCCO5)c4n3)cc2OC2CCC2)CC1]

or pharmaceutically acceptable salts thereof.
   Embodiment 21. A compound selected from:

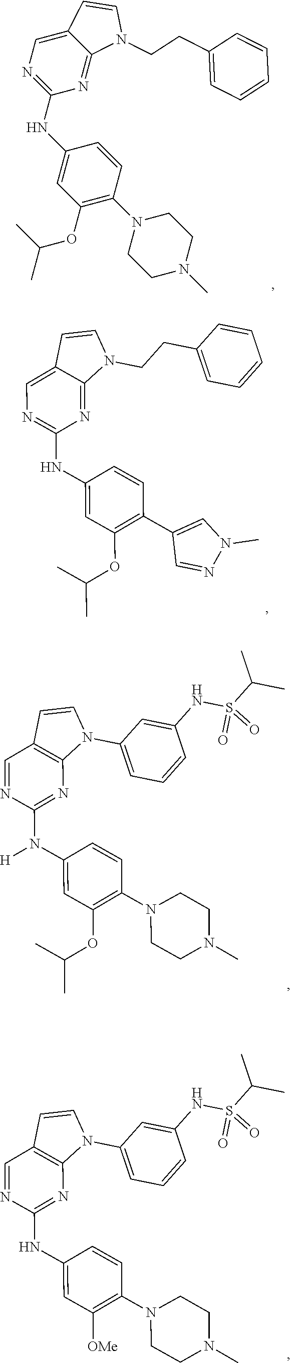

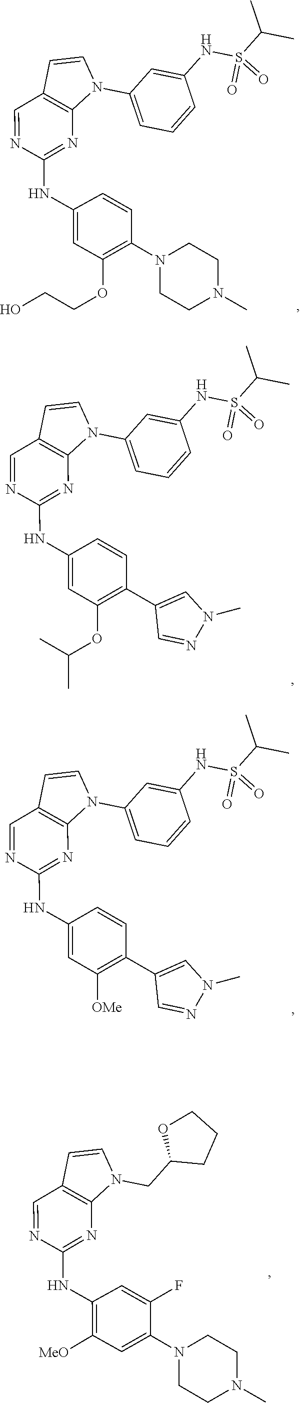

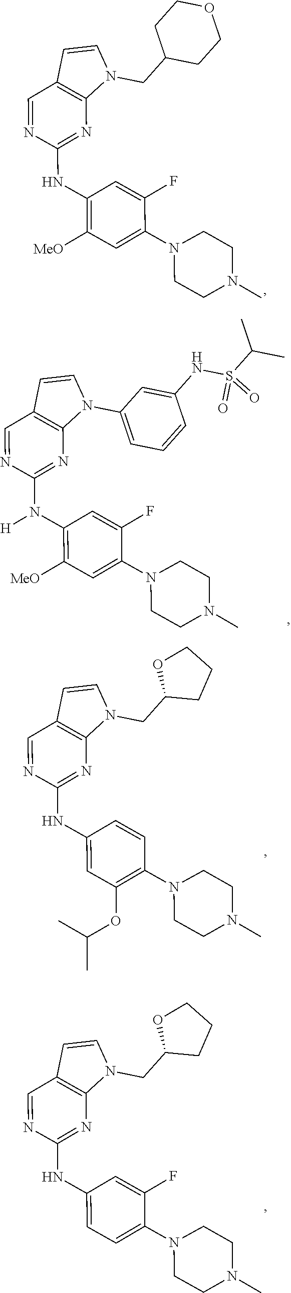

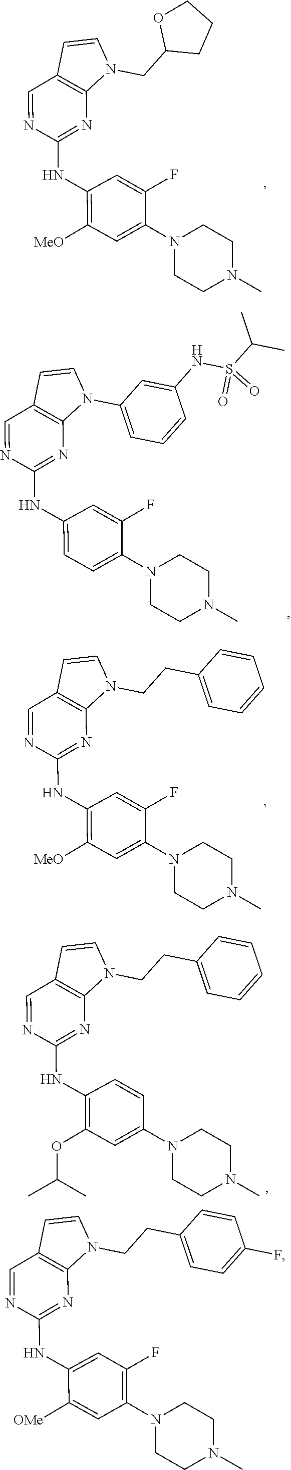

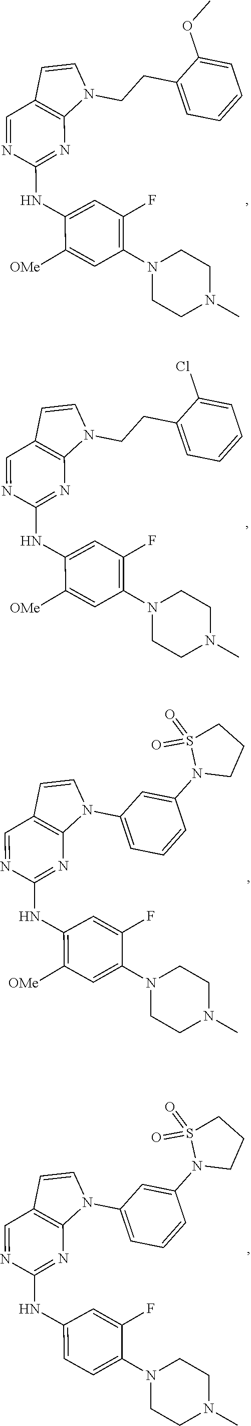 [SMILES: COc1ccccc1CCn1ccc2cnc(Nc3cc(F)c(N4CCN(C)CC4)cc3OC)nc21 ; COc1cc(N2CCN(C)CC2)c(F)cc1Nc1ncc2ccn(CCc3ccccc3Cl)c2n1 ; COc1cc(N2CCN(C)CC2)c(F)cc1Nc1ncc2ccn(-c3cccc(N4CCCS4(=O)=O)c3)c2n1 ; CN1CCN(c2ccc(Nc3ncc4ccn(-c5cccc(N6CCCS6(=O)=O)c5)c4n3)cc2F)CC1]

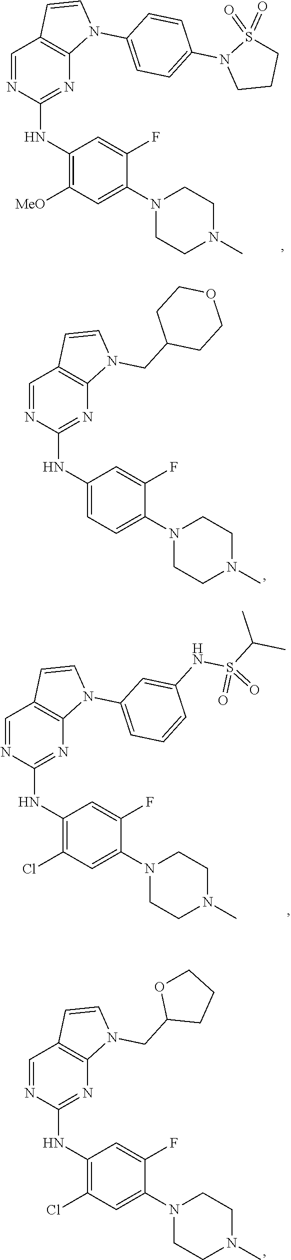

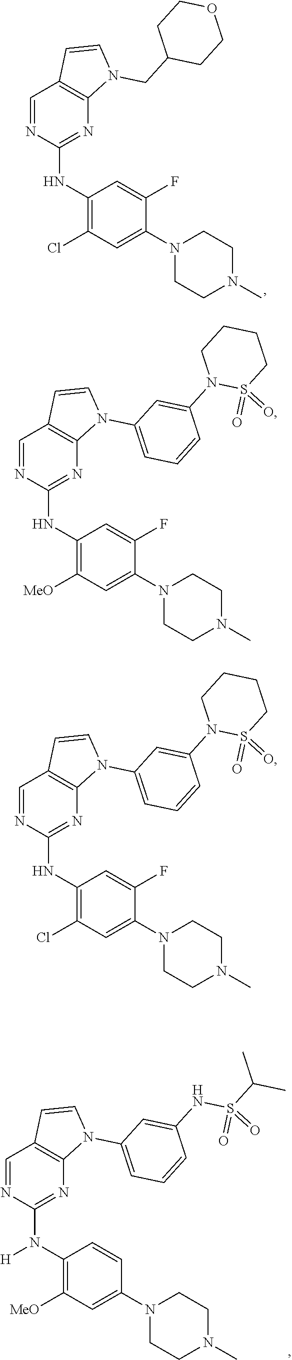

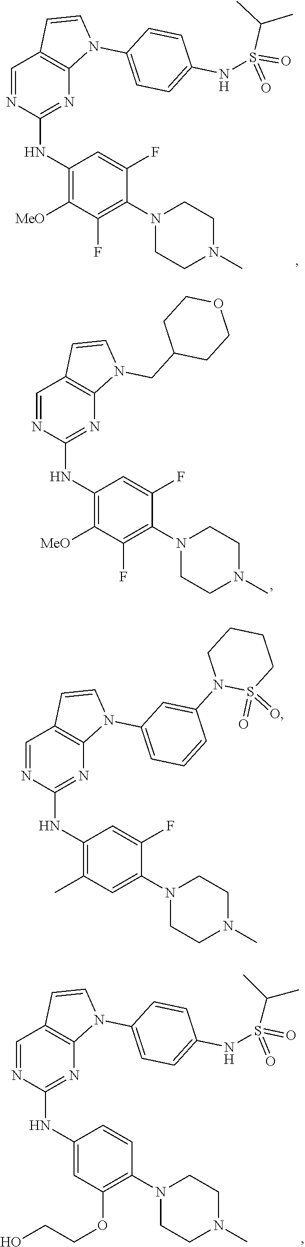

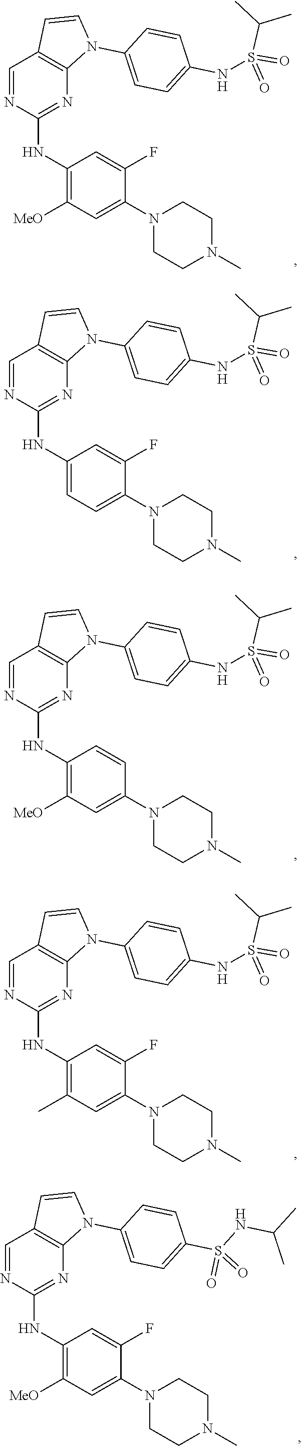

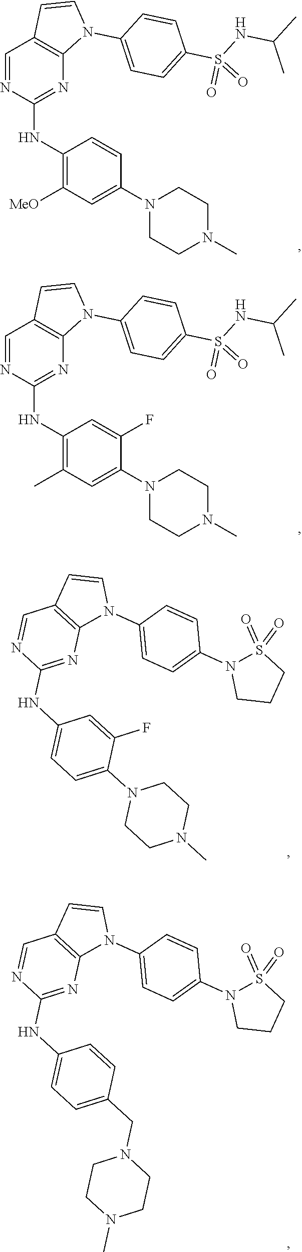 [SMILES: COc1cc(N2CCN(C)CC2)ccc1Nc1ncc2ccn(-c3ccc(S(=O)(=O)NC(C)C)cc3)c2n1 ; Cc1cc(N2CCN(C)CC2)c(F)cc1Nc1ncc2ccn(-c3ccc(S(=O)(=O)NC(C)C)cc3)c2n1 ; CN1CCN(c2ccc(Nc3ncc4ccn(-c5ccc(N6CCCS6(=O)=O)cc5)c4n3)cc2F)CC1 ; CN1CCN(Cc2ccc(Nc3ncc4ccn(-c5ccc(N6CCCS6(=O)=O)cc5)c4n3)cc2)CC1]

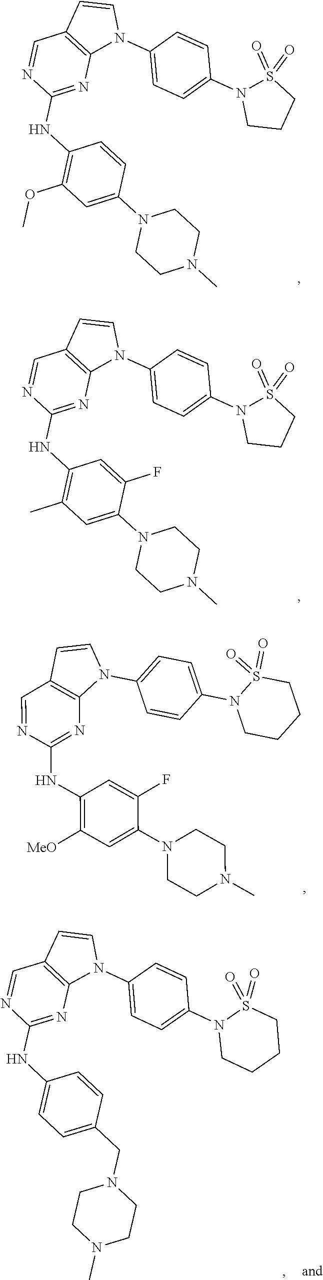 [SMILES: COc1cc(N2CCN(C)CC2)ccc1Nc1ncc2ccn(-c3ccc(N4CCCS4(=O)=O)cc3)c2n1 ; Cc1cc(N2CCN(C)CC2)c(F)cc1Nc1ncc2ccn(-c3ccc(N4CCCS4(=O)=O)cc3)c2n1 ; COc1cc(N2CCN(C)CC2)c(F)cc1Nc1ncc2ccn(-c3ccc(N4CCCCS4(=O)=O)cc3)c2n1 ; CN1CCN(Cc2ccc(Nc3ncc4ccn(-c5ccc(N6CCCCS6(=O)=O)cc5)c4n3)cc2)CC1]

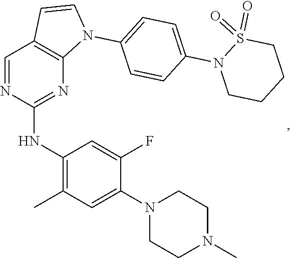 [SMILES: Cc1cc(N2CCN(C)CC2)c(F)cc1Nc1ncc2ccn(-c3ccc(N4CCCCS4(=O)=O)cc3)c2n1]

or pharmaceutically acceptable salts thereof.
   Embodiment 22. A pharmaceutical composition comprising a compound of any one of embodiments 1-21, or a pharmaceutically acceptable salt thereof, and a pharmaceutically acceptable carrier or excipient.
   Embodiment 23. A method of treating a cancer in a subject in need thereof comprising administering to the subject a therapeutically effective amount of a compound of any one of embodiments 1-21, or a pharmaceutically acceptable salt thereof, or a pharmaceutical composition of embodiment 22.
   Embodiment 24. The method of embodiment 23, further comprising an additional therapeutic agent.
   Embodiment 25. The method of embodiment 24, wherein the additional therapeutic agent comprises an anti-cancer agent or an anti-inflammatory agent.
   Embodiment 26. The method of any one of embodiments 23-25, further comprising administering an effective amount of ionizing radiation to the subject.
   Embodiment 27. A method of killing a tumor cell comprising contacting the tumor cell with an effective amount of a compound of any one of embodiments 1-21, or a pharmaceutically acceptable salt thereof, or a composition of embodiment 22.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are put forth so as to provide those of ordinary skill in the art with a complete disclosure and description of how the compounds, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

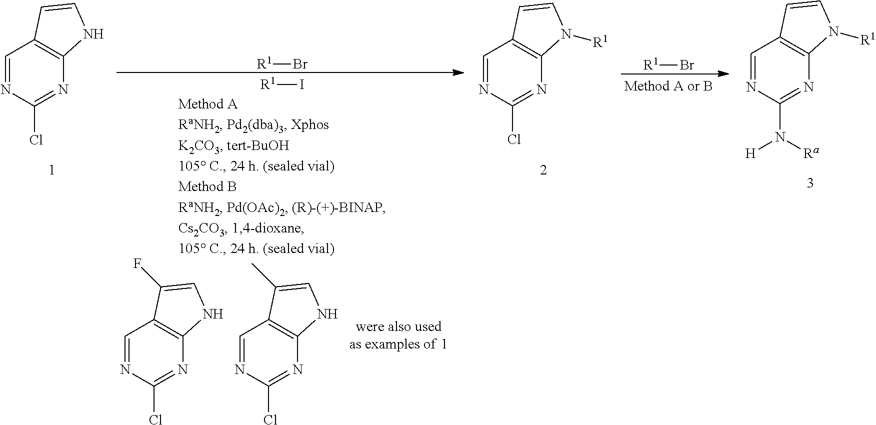

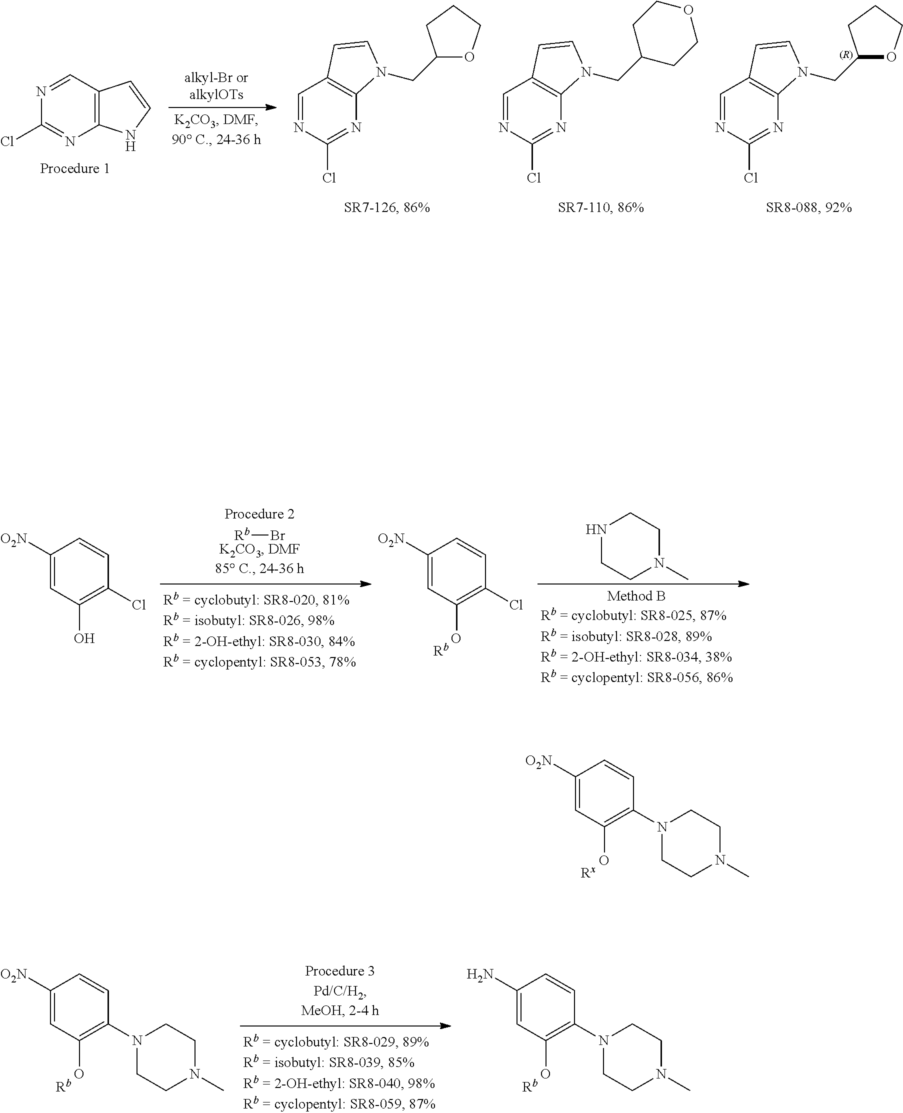

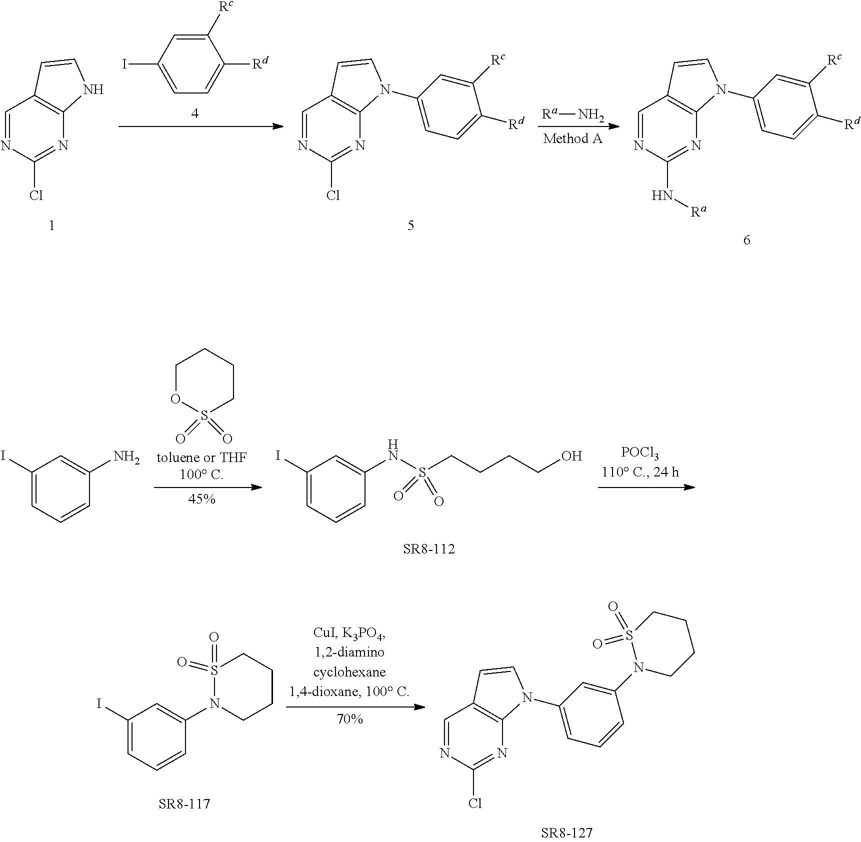

ACK1 inhibitors were prepared by addition of alkyl and aryl groups to the 7 position of 2-chloropyrrolopyrimidine 1, followed by substitution of the 2-chloro group of intermediate 2 with a functionalized aniline (R²NH₂), using Buchwald-Hartwig amination (see Dorel, R.; Grugel, C. P.; Haydl, A. M., The Buchwald-Hartwig Amination After 25 Years. Angewandte Chemie (International ed. in English) 2019, 58 (48), 17118-17129) methods A or B to provide pyrrolopyrimidines of type 3. The anilines (R²NH₂) were commercially available or prepared by methods such as alkylation of 2-chloro-5-nitrophenol (procedure 2) followed by Buchwald-Hartwig amination (Method B) and finally reduction (procedure 3). Arylation of the N-7 position of 2-chloropyrrolopyrimidine 1 can be achieved by reaction of an iodophenyl derivative 4 by copper catalysis using reported methods to provide 2-chloro-7-arylpyrrolopyrimdines 5 (see Oeser, P.; Koudelka, J.; Petrenko, A.; Tobrman, T., Recent Progress Concerning the N-Arylation of Indoles. Molecules (Basel, Switzerland) 2021, 26 (16)). Reaction of 5 with substituted anilines provides the N-7-aryl derivatives 6. Iodoarenes bearing cyclic sultams were prepared by reaction with butylsultone as reported (see Valente, C.; Guedes, R. C.; Moreira, R.; Iley, J.; Gut, J.; Rosenthal, P. J., Dipeptide vinyl sultams: synthesis via the Wittig-Horner reaction and activity against papain, falcipain-2 and Plasmodium falciparum. Bioorganic & medicinal chemistry letters 2006, 16 (15), 4115-9).

### General Procedure 1: Alkylation of 2-chloropyrrolo(2,3-d)pyrimidine 1

2-Chloropyrrolo(2,3-d)pyrimidine (1.00 g, 6.51 mmol) and potassium carbonate (2.25 g, 16.28 mmol) were dissolved in dry DMF (10 mL) in a pressure flask under Ar. Alkyl bromide or alkyl tosylate (1.1 eq) was added to the mixture and stirred at rt or 80° C. for 36-48 h. The mixture was cooled to room temperature and diluted with EtOAc (30 mL). The organic layer was washed with water (2×25 mL) and brine (1×25 mL) and dried over an h. Na₂SO₄. The solvent was removed under vacuum to afford a brown solid. Purification by trituration (DCM/hexane) or column chromatography (0-50% gradient elution, EtOAc:hexane) afforded the corresponding N-alkylated pyrrolopyrimidine as a solid.

### Buchwald-Hartwig Amination Reaction.¹ General Procedure A:

N-alkylated 2-chloro-7-hydro-pyrrolo (2,3-d)pyrimidine 2 (0.050 g, 1.0 eq.), substituted aniline (0.9-1.0 eq.), Xphos (20 mol %), and K₂CO₃ (1.5 eq.) were placed in a microwave vial (5 mL). The mixture was dissolved in tert-butanol (2.0 mL) and degassed for 10 mins using Ar stream through the solvent. Pd₂(dba)₃ (10 mol %) was added to the mixture. The vial was sealed and heated at 105° C. for 16-24 h. The mixture was diluted with EtOAc (25 mL) and washed with sat. NH₄Cl (15 mL) and brine (15 mL). The organic layer was dried (Na₂SO₄) and concentrated under vacuum. Purification by column chromatography (0-15% gradient elution, MeOH:DCM) afforded the product as a foam or solid (29-56% yield).

### Buchwald-Hartwig Amination Reaction.¹ General Procedure B:

N-alkylated 2-chloro-7-hydro-pyrrolo(2,3-d)pyrimidine (2, 0.050 g, 1.0 eq.), aniline derivative (0.9-1.0 eq.), (R)-(+)-BINAP (10 mol %), and K₂CO₃ (1.5 eq.) were placed in a microwave vial (5 mL). The mixture was dissolved in 1,4-dioxane (2.0 mL) and degassed for 10 mins using Ar stream through the solvent. Pd₂(OAc)₂ (10 mol %) was added to the mixture. The vial was sealed and placed at 105° C. for 20-24 h. The work-up and purification was the same as in general procedure A providing the product as a foam or solid (55-87% yield).

General procedure 2: Alkylation of 2-chloro-4-nitrophenol. 2-Chloro-4-nitrophenol (1.00 g, 5.76 mmol) and potassium carbonate (1.59 g, 11.56 mmol) were dissolved in dry DMF (10 mL) in a pressure flask under Ar. The alkyl bromide (1.1-2.2 eq) was added to the mixture and stirred at 85° C. for 24-36 h. The mixture was cooled to rt and diluted with EtOAc (30 mL). The organic layer was washed with water (2×25 mL) and brine (1×25 mL) and dried (Na₂SO₄). The solvent was removed under vacuum to afford yellowish brown solid. Purification by trituration (DCM/hexane) or column chromatography (0-50% gradient elution, EtOAc:hexane) afforded the corresponding O-alkylated product as a solid.

General procedure 3: Reduction of nitroarenes by catalytic hydrogenation. Two neck round bottom flask was charged with stirring bar and purged with argon. Required 10% Pd/C (100 mg/mmol) was added into the reaction flask under argon atmosphere. Methanol (2.5 mL/mmol) was added into the flask carefully along the side wall under argon. Then nitro substrate (1 mmol) was added into the mixture as a solids or solution (in methanol, 1-2 mL) carefully. The mixture was placed briefly under vacuum and then purged with argon with stirring. Finally, a hydrogen balloon was attached to the flask through a septum and placed briefly under vacuum and purged with hydrogen. The evacuation and purging steps were repeated 2 more times. The reaction was monitored at rt until the reduction completed. The mixture was filtered through Celite and rinsed with EtOAc (50 mL). The resulting filtrate was concentrated under reduced pressure. Purification column chromatography (0-25% gradient elution, MeOH:DCM) afforded the aniline derivatives.

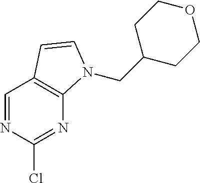 [SMILES: Clc1ncc2ccn(CC3CCOCC3)c2n1]

### 2-Chloro-7-((tetrahydro-2H-pyran-4-yl)methyl)-7H-pyrrolo[2,3-d]pyrimidine (SR7-110)

SR7-110 was obtained as a solid (1.408 g, 86% yield) from 4-bromoethylpyran (1.282 g, 7.160 mmol) by following general procedure 1. ¹H NMR (500 MHz, DMSO) δ 8.93 (s, 1H), 7.69 (d, J=3.5 Hz, 1H), 6.70 (d, J=3.5 Hz, 1H), 4.12 (d, J=7.4 Hz, 2H), 3.82 (ddd, J=11.6, 4.6, 2.0 Hz, 2H), 3.23 (td, J=11.6, 2.3 Hz, 2H), 2.11 (ttt, J=11.4, 7.6, 4.0 Hz, 1H), 1.36 (ddd, J=13.0, 4.3, 2.1 Hz, 2H), 1.26 (dtd, J=13.2, 11.6, 4.5 Hz, 2H); ¹³C NMR (126 MHz, DMSO) δ 152.6, 152.0, 151.8, 132.4, 118.0, 100.1, 66.9, 49.8, 35.7, 30.5.

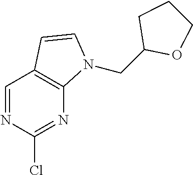 [SMILES: Clc1ncc2ccn(CC3CCCO3)c2n1]

2-Chloro-7-((tetrahydrofuran-2-yl)methyl)-7H-pyrrolo[2,3-d]pyrimidine (SR7-126). SR7-126 was obtained as a gum (1.325 g, 86% yield) from tertrahydrofurfural bromide (1,074 g, 6.510 mmol) by following general procedure 1. ¹H NMR (500 MHz, DMSO) δ 8.93 (s, 1H), 7.66 (d, J=3.6 Hz, 1H), 6.68 (d, J=3.5 Hz, 1H), 4.30 (m, 1H), 4.25 (m, 1H), 4.20 (m, 1H), 3.77 (m, 1H), 3.63 (ddd, J=8.2, 7.2, 6.3 Hz, 1H), 2.01-1.91 (m, 1H), 1.84-1.73 (m, 2H), 1.57 (m, 1H); ¹³C NMR (126 MHz, DMSO) δ 152.6, 152.0, 151.7, 132.6, 118.1, 100.0, 77.3. 67.8, 48.1, 28.9, 25.5.

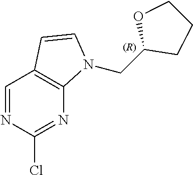

(R)-2-Chloro-7-((tetrahydrofuran-2-yl)methyl)-7H-pyrrolo[2,3-d]pyrimidine (SR8-088). SR8-088 was obtained as a thick oil (1.42 g, 92% yield) from (R)-(tetrahydrofuran-2-yl)methyl 4-methylbenzenesulfonate (prepared according to the reported procedure in Grubb, L. M.; Branchaud, B. P., Complete Retention of Configuration in a Cobaloxime π-Cation-Mediated Cyclization of an (ω-Hydroxy-β-hydroxyalkyl)cobaloxime. The Journal of Organic Chemistry 1997, 62 (2), 242-243) (1.84 g, 7.16 mmol) by following general procedure 1. ¹H NMR (500 MHz, DMSO) δ 8.93 (s, 1H), 7.66 (d, J=3.6 Hz, 1H), 6.68 (d, J=3.6 Hz, 1H), 4.31-4.24 (m, 2H), 4.23-4.17 (m, 1H), 3.77 (dd, J=14.9, 6.9 Hz, 1H), 3.63 (dd, J=14.1, 7.7 Hz, 1H), 2.02-1.89 (m, 1H), 1.84-1.70 (m, 2H), 1.61-1.51 (m, 1H). HPLC-MS (ESI+): m/z 237.9 [100% (M+H)⁺].

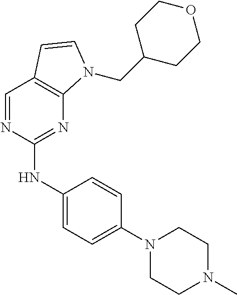 [SMILES: CN1CCN(c2ccc(Nc3ncc4ccn(CC5CCOCC5)c4n3)cc2)CC1]

N-(4-(4-Methylpiperazin-1-yl)phenyl)-7-((tetrahydro-2H-pyran-4-yl)methyl)-7H-pyrrolo[2,3-d]pyrimidin-2-amine (SR7-121). SR7-121 was obtained as a foam (0.028 g, 35% yield) from SR7-110 (0.050 g, 0.199 mmol) and 4(4-methylpiperazine)aniline (0.033 g, 0.199 mmol) by following the general procedure A. HPLC: >99% [t_{R}=13.0 min, gradient 5-95% MeOH and water (with 0.1% TFA), 20 min]. ¹H NMR (500 MHz, DMSO) δ 9.11 (s, 1H), 8.63 (s, 1H), 7.72 (d, J=9.1 Hz, 2H), 7.21 (d, J=3.6 Hz, 1H), 6.89 (d, J=9.1 Hz, 2H), 6.40 (d, J=3.6 Hz, 1H), 4.03 (d, J=7.1 Hz, 2H), 3.84 (ddd, J=11.4, 4.5, 2.0 Hz, 2H), 3.24 (td, J=11.7, 2.2 Hz, 2H), 3.06 (t, J=5.0 Hz, 4H), 2.46 (t, J=4.9 Hz, 4H), 2.23 (s, 3H), 2.15 (m, 1H), 1.43 (ddd, J=12.7, 4.1, 1.9 Hz, 2H), 1.29 (dtd, J=13.3, 11.7, 4.6 Hz, 2H). ¹³C NMR (126 MHz, DMSO) δ 156.6, 152.2, 150.8, 145.9, 134.3, 127.4, 119.6, 116.5, 112.0, 99.7, 67.0, 55.3, 49.6, 49.5, 46.3, 35.9, 30.8. HRMS (ESI+): m/z calcd for C₂₃H₃₁N₆O (M+H)⁺ 407.2554, found 407.2558 m/z calcd for C₂₃H₃₀N₆ONa (M+Na)⁺ 429.2373, 429.2376; HPLC-MS (ESI+): m/z 407.2 [20% (M+H)⁺], 204.2 [90%, ((M+2H)²⁺/2)].

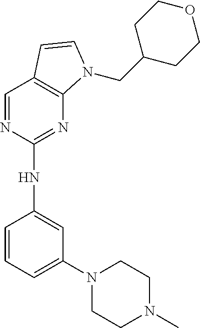 [SMILES: CN1CCN(c2cccc(Nc3ncc4ccn(CC5CCOCC5)c4n3)c2)CC1]

N-(3-(4-Methylpiperazin-1-yl)phenyl)-7-((tetrahydro-2H-pyran-4-yl)methyl)-7H-pyrrolo[2,3-d]pyrimidin-2-amine (SR7-122). SR7-122 was obtained as a foam (0.041 g, 51% yield) from SR7-110 (0.050 g, 0.199 mmol) and 3(4-methylpiperazine-1-yl)aniline (0.038 g, 0.199 mmol) by following the general procedure A. HPLC: >99% [t_{R}=13.6 min, gradient 5-95% MeOH and water (with 0.1% TFA), 20 min]. ¹H NMR (500 MHz, DMSO) δ 9.23 (s, 1H), 8.67 (s, 1H), 7.79 (t, J=2.2 Hz, 1H), 7.26 (d, J=3.5 Hz, 1H), 7.15 (ddd, J=8.1, 2.0, 0.9 Hz, 1H), 7.08 (t, J=8.0 Hz, 1H), 6.51 (ddd, J=8.1, 2.4, 0.9 Hz, 1H), 6.43 (d, J=3.5 Hz, 1H), 4.06 (d, J=7.3 Hz, 2H), 3.83 (ddd, J=11.5, 4.5, 1.9 Hz, 2H), 3.23 (d, J=2.1 Hz, 2H), 3.16 (t, J=5.0 Hz, 4H), 2.48 (bs, 4H), 2.24 (s, 3H), 2.18-2.08 (m, 1H), 1.39 (m, 2H), 1.34-1.18 (m, 2H); ¹³C NMR (126 MHz, DMSO) δ 156.3, 152.0, 151.9, 150.9, 142.5, 129.1, 127.5, 112.2, 109.9, 108.7, 105.7, 99.8, 66.9, 55.2, 49.4, 48.9, 46.3, 35.9, 30.7. HRMS (ESI+): m/z calcd for C₂₃H₃₁N₆O (M+H)⁺ 407.2554, found 407.2552 m/z calcd for C₂₃H₃₀N₆ONa (M+Na)⁺ 429.2373, found 429.2377; HPLC-MS (ESI+): m/z 407.2 [20% (M+H)⁺], 204.2 [90%, ((M+2H)²⁺/2)].

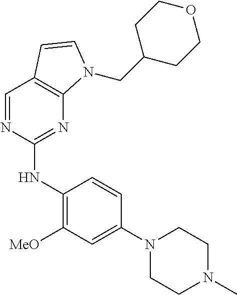 [SMILES: COc1cc(N2CCN(C)CC2)ccc1Nc1ncc2ccn(CC3CCOCC3)c2n1]

N-(2-Methoxy-4-(4-methylpiperazin-1-yl)phenyl)-7-((tetrahydro-2H-pyran-4-yl)methyl)-7H-pyrrolo[2,3-d]pyrimidin-2-amine (SR7-124). SR7-124 was obtained as a foam (0.023 g, 29% yield) from SR7-110 (0.050 g, 0.199 mmol) and 2-methoxy-4-(4-methylpiperazine-1-yl)aniline (0.040 g, 0.179 mmol) by following the general procedure A. HPLC: >99% [t_{R}=13.3 min, gradient 5-95% MeOH and water (with 0.1% TFA), 20 min]. ¹H NMR (500 MHz, DMSO-d6) δ 8.61 (s, 1H), 8.21 (d, J=8.8 Hz, 1H), 7.55 (s, 1H), 7.22 (d, J=3.5 Hz, 1H), 6.65 (d, J=2.6 Hz, 1H), 6.51 (dd, J=8.8, 2.6 Hz, 1H), 6.41 (d, J=3.5 Hz, 1H), 4.01 (d, J=7.2 Hz, 2H), 3.86 (s, 3H), 3.82 (ddd, J=11.4, 4.4, 2.0 Hz, 2H), 3.23 (td, J=11.7, 2.2 Hz, 3H), 3.10 (t, J=4.9 Hz, 4H), 2.47 (m, 4H), 2.23 (s, 3H), 2.17-2.06 (m, 1H), 1.41 (m, 2H), 1.36-1.20 (m, 2H); ¹³C NMR (126 MHz, DMSO) δ 156.4, 152.2, 150.9, 149.6, 147.1, 127.6, 122.5, 119.9, 112.3, 107.4, 100.7, 99.7, 67.0, 56.2, 55.2, 49.5, 46.3, 35.9, 30.7. HRMS (ESI+): m/z calcd for C₂₄H₃₃N₆O₂ (M+H)⁺ 437.2660, found 437.2663 m/z calcd for C₂₄H₃₂N₆O₂Na (M+Na)⁺ 459.2479, found 459.2483; HPLC-MS (ESI+): m/z 437.2 [10% (M+H)⁺], 459.2 [90%, ((M+2H)²⁺/2)].

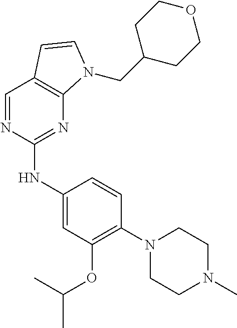 [SMILES: CC(C)Oc1cc(Nc2ncc3ccn(CC4CCOCC4)c3n2)ccc1N1CCN(C)CC1]

N-(3-isoPropoxy-4-(4-methylpiperazin-1-yl)phenyl)-7-((tetrahydro-2H-pyran-4-yl)methyl)-7H-pyrrolo[2,3-d]pyrimidin-2-amine (SR7-152). SR7-152 was obtained as an yellow foam (0.026 g, 31% yield) from SR7-110 (0.050 g, 0.198 mmol) and 3-isopropoxy-4-(4-methylpiperazin-1-yl)aniline (0.044 g, 0.179 mmol) by following the general procedure A. HPLC: >99% [t_{R}=14.6 min, gradient 5-95% MeOH and water (with 0.1% TFA), 20 min]. ¹H NMR (500 MHz, DMSO) δ 9.19 (s, 1H), 8.65 (s, 1H), 7.74 (d, J=2.4 Hz, 1H), 7.28-7.21 (m, 2H), 6.81 (d, J=8.6 Hz, 1H), 6.42 (d, J=3.6 Hz, 1H), 4.62 (m, 1H), 4.05 (d, J=7.3 Hz, 2H), 3.83 (m, 2H), 3.24 (td, J=11.8, 2.1 Hz, 2H), 2.94 (bs, 4H), 2.49-2.34 (bs, 4H), 2.23 (s, 3H), 2.14 (m, 1H), 1.40 (m, 2H), 1.31 (d, J=6.0 Hz, 6H), 1.26 (m, 2H); ¹³C NMR (126 MHz, DMSO) δ 156.4, 152.1, 150.9, 150.2, 137.1, 136.7, 127.3, 118.7, 112.1, 111.5, 107.9, 99.8, 70.1, 67.0, 55.6, 50.7, 49.3, 46.4, 35.9, 30.7, 22.6. HRMS (ESI+): m/z calcd for C₂₆H₃₇N₆O₂ (M+H)⁺ 465.2973, found 465.2965 m/z calcd for C₂₆H₃₆N₆O₂Na (M+Na)⁺ 487.2792, found 487.2761; HPLC-MS (ESI+): m/z 465.6 [40% (M+H)⁺], 233.4 [100%, ((M+2H)²⁺/2)].

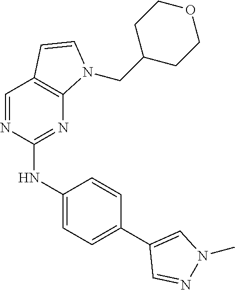 [SMILES: Cn1cc(-c2ccc(Nc3ncc4ccn(CC5CCOCC5)c4n3)cc2)cn1]

N-(4-(1-Methyl-1H-pyrazol-4-yl)phenyl)-7-((tetrahydro-2H-pyran-4-yl)methyl)-7H-pyrrolo[2,3-d]pyrimidin-2-amine (SR7-161). SR7-161 was obtained a solid (0.024 g, 35% yield) from SR7-110 (0.050 g, 0.198 mmol) and 4-(1-methyl-1H-pyrazol-4-yl)aniline (0.031 g, 0.178 mmol) by following the general procedure B. HPLC: >99% [t_{R}=16.7 min, gradient 5-95% MeOH and water (with 0.1% TFA), 20 min]. ¹H NMR (500 MHz, DMSO) δ 9.40 (s, 1H), 8.69 (s, 1H), 8.04 (d, J=0.8 Hz, 1H), 7.88 (d, J=8.7 Hz, 2H), 7.79 (d, J=0.8 Hz, 1H), 7.47 (d, J=8.7 Hz, 2H), 7.27 (d, J=3.6 Hz, 1H), 6.44 (d, J=3.5 Hz, 1H), 4.07 (d, J=7.2 Hz, 2H), 3.86 (s, 3H), 3.84 (dd, J=4.6, 1.9 Hz, 1H), 3.25 (td, J=11.7, 2.2 Hz, 2H), 2.22-2.11 (m, 1H), 1.44 (m, 2H), 1.37-1.25 (m, 2H); ¹³C NMR (126 MHz, DMSO) δ 156.2, 152.1, 150.8, 140.0, 136.0, 127.8, 127.4, 125.6, 125.4, 122.6, 118.8, 112.4, 99.8, 67.0, 49.6, 39.1, 35.9, 30.8. HRMS (ESI+): m/z calcd for C₂₂H₂₄N₆O (M+H)⁺ 389.2084, found 389.2079 m/z calcd for C₂₂H₂₄N₆ONa (M+Na)⁺ 411.1904, found 411.1916; HPLC-MS (ESI+): m/z 389.2 [100% (M+H)⁺], 195.2 [20%, ((M+2H)²⁺/2)].

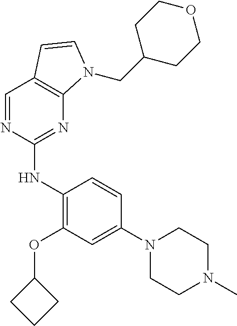 [SMILES: CN1CCN(c2ccc(Nc3ncc4ccn(CC5CCOCC5)c4n3)c(OC3CCC3)c2)CC1]

N-(2-Cyclobutoxy-4-(4-methylpiperazin-1-yl)phenyl)-7-((tetrahydro-2H-pyran-4-yl)methyl)-7H-pyrrolo[2,3-d]pyrimidin-2-amine (SR7-182). SR7-182 was obtained as a foam (0.047 g, 55% yield) from SR7-110 (0.050 g, 0.199 mmol) and 2-cyclobutoxy-4-(4-methylpiperazin-1-yl)aniline (0.047 g, 0.179 mmol) by following the general procedure B. HPLC: >99% [t_{R}=14.3 min, gradient 5-95% MeOH and water (with 0.1% TFA), 20 min]. ¹H NMR (500 MHz, DMSO) δ 8.62 (s, 1H), 8.28 (d, J=8.8 Hz, 1H), 7.51 (s, 1H), 7.23 (d, J=3.5 Hz, 1H), 6.51 (dd, J=8.9, 2.6 Hz, 1H), 6.46 (d, J=2.6 Hz, 1H), 6.42 (d, J=3.5 Hz, 1H), 4.84-4.74 (m, 1H), 4.03 (d, J=7.2 Hz, 2H), 3.82 (ddd, J=11.4, 4.5, 2.0 Hz, 2H), 3.23 (td, J=11.6, 2.2 Hz, 2H), 3.07 (t, J=5.0 Hz, 4H), 2.45 (m, 6H), 2.23 (s, 3H), 2.17-2.04 (m, 3H), 1.79 (m, 1H), 1.64 (m, 1H), 1.42 (m, 2H), 1.35-1.20 (m, 2H); ¹³C NMR (126 MHz, DMSO) δ 156.2, 152.2, 146.9, 146.8, 127.6, 122.7, 119.6, 112.3, 107.7, 101.9, 99.8, 71.6, 67.0, 55.2, 49.5, 49.4, 46.2, 36.0, 3087, 30.7, 13.2. HRMS (ESI+): m/z calcd for C₂₇H₃₇N₆O₂ (M+H)⁺ 477.2973, found 477.2963 m/z calcd for C₂₇H₃₆N₆O₂Na (M+Na)⁺ 499.2792, found 499.2787; HPLC-MS (ESI+): m/z 477.3 [30% (M+H)⁺], 239.2 [100%, ((M+2H)²⁺/2)].

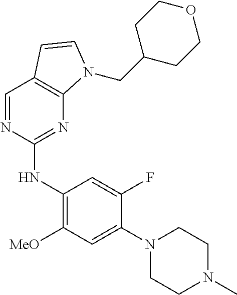 [SMILES: COc1cc(N2CCN(C)CC2)c(F)cc1Nc1ncc2ccn(CC3CCOCC3)c2n1]

N-(5-Fluoro-2-methoxy-4-(4-methylpiperazin-1-yl)phenyl)-7-((tetrahydro-2H-pyran-4-yl)methyl)-7H-pyrrolo[2,3-d]pyrimidin-2-amine (SR8-092). SR8-092 was obtained as a foam (0.044 g, 54% yield) from SR7-110 (0.050 g, 0.199 mmol) and 3-fluoro-6-methoxy-4-(4-methylpiperazin-1-yl)aniline (0.043 g, 0.179 mmol) by following the general procedure B. HPLC: >99% [t_{R}=14.6 min, gradient 5-95% MeOH and water (with 0.1% TFA), 20 min]. ¹H NMR (500 MHz, DMSO) δ 8.69 (s, 1H), 8.41 (d, J=15.3 Hz, 1H), 7.68 (d, J=1.3 Hz, 1H), 7.30 (d, J=3.6 Hz, 1H), 6.72 (d, J=8.2 Hz, 1H), 6.46 (d, J=3.5 Hz, 1H), 4.05 (d, J=7.2 Hz, 2H), 3.90 (s, 3H), 3.84 (ddd, J=11.3, 4.4, 2.0 Hz, 2H), 3.24 (m, 2H), 3.02 (t, J=4.9 Hz, 4H), 2.60-2.52 (m, 4H), 2.26 (s, 3H), 2.23-2.11 (m, 1H), 1.44 (m, 2H), 1.35-1.22 (m, 2H); ¹⁹F NMR (471 MHz, DMSO) δ −131.33 (dd, J=15.4, 8.3 Hz); ¹³C NMR (126 MHz, DMSO) δ 155.5, 151.9, 150.0, 148.1, 144.5, 128.3, 124.5, 112.9, 107.0, 106.8, 103.3, 99.8, 66.9, 56.9, 55.2, 50.8, 49.8, 46.2, 35.8, 30.8. HRMS (ESI+): m/z calcd for C₂₄H₃₂FN₆O₂(M+H)⁺ 455.2565, found 455.2565. m/z calcd for C₂₄H₃₁FN₆O₂Na (M+Na)⁺ 477.2385, found 477.2361; HPLC-MS (ESI+): m/z 455.1 [70% (M+H)⁺], 228.1 [100%, ((M+2H)²⁺/2)].

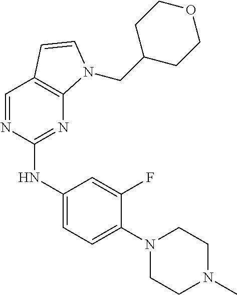 [SMILES: CN1CCN(c2ccc(Nc3ncc4ccn(CC5CCOCC5)c4n3)cc2F)CC1]

N-(3-Fluoro-4-(4-methylpiperazin-1-yl)phenyl)-7-((tetrahydro-2H-pyran-4-yl)methyl)-7H-pyrrolo[2,3-d]pyrimidin-2-amine (SR8-098). SR8-098 was obtained as a foam (0.048 g, 64% yield) from SR7-110 (0.050 g, 0.199 mmol) and 3-fluoro-4-(4-methylpiperazin-1-yl)aniline (0.037 g, 0.179 mmol) by following the general procedure B. HPLC: >98% [t_{R}=14.0 min, gradient 5-95% MeOH and water (with 0.1% TFA), 20 min]. ¹H NMR (600 MHz, DMSO) δ 9.44 (s, 1H), 8.68 (s, 1H), 7.94 (dd, J=15.9, 2.5 Hz, 1H), 7.44 (dd, J=8.8, 2.4 Hz, 1H), 7.27 (d, J=3.6 Hz, 1H), 6.97 (dd, J=10.1, 8.8 Hz, 1H), 6.44 (d, J=3.5 Hz, 1H), 4.05 (d, J=7.2 Hz, 2H), 3.84 (m, 2H), 3.24 (td, J=11.7, 2.1 Hz, 2H), 2.96 (bs, 4H), 2.51 (bs, 4H), 2.26 (s, 3H), 2.17 (m, 1H), 1.44 (m, 2H), 1.35-1.24 (m, 2H); ¹⁹F NMR (471 MHz, DMSO) δ −122.40 (dd, J=16.0, 10.1 Hz); ¹³C NMR (126 MHz, DMSO) δ 156.3, 156.0, 154.4, 151.9, 150.9, 137.5, 133.32, 128.0, 119.8, 114.3, 112.5, 106.6, 106.4, 99.7, 67.0, 55.2, 50.9, 49.7, 46.2, 35.9, 30.8. HRMS (ESI+): m/z calcd for C₂₃H₃₀FN₆O (M+H)⁺ 425.2460, found 425.2452. m/z calcd for C₂₃H₂₉FN₆ONa (M+Na)⁺ 447.2279, found 447.2272; HPLC-MS (ESI+): m/z 425.1 [80% (M+H)⁺], 213.1 [100%, ((M+2H)²⁺/2)].

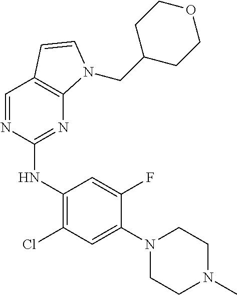 [SMILES: CN1CCN(c2cc(Cl)c(Nc3ncc4ccn(CC5CCOCC5)c4n3)cc2F)CC1]

N-(2-Chloro-5-fluoro-4-(4-methylpiperazin-1-yl)phenyl)-7-((tetrahydro-2H-pyran-4-yl)methyl)-7H-pyrrolo[2,3-d]pyrimidin-2-amine (SR8-124). SR8-124 was obtained as a foam (0.044 g, 54% yield) from SR7-110 (0.050 g, 0.199 mmol) and SR8-120 (0.044 g, 0.179 mmol) by following the general procedure B. HPLC: >99% [t_{R}=15.4 min, gradient 5-95% MeOH and water (with 0.1% TFA), 20 min]. ¹H NMR (500 MHz, DMSO) δ 8.70 (s, 1H), 8.17 (d, J=15.8 Hz, 1H), 8.13 (s, 1H), 7.30 (d, J=3.5 Hz, 1H), 7.11 (d, J=8.9 Hz, 1H), 6.46 (d, J=3.6 Hz, 1H), 4.01 (d, J=7.1 Hz, 2H), 3.83 (ddd, J=11.6, 4.4, 2.0 Hz, 2H), 3.24 (td, J=11.6, 2.1 Hz, 2H), 3.00 (t, J=4.8 Hz, 4H), 2.48 (m, 4H), 2.24 (s, 3H), 2.20-2.08 (m, 1H), 1.42 (m, 2H), 1.34-1.19 (m, 2H); ¹⁹F NMR (471 MHz, DMSO) δ −122.88 (dd, J=15.1, 8.9 Hz); HRMS (ESI+): m/z calcd for C₂₃H₂₉ClFN₆O (M+H)⁺ 459.2070, found 429.2055 m/z calcd for C₂₃H₂₈ClFN₆ONa (M+Na)⁺ 481.1889, found 481.1885; HPLC-MS (ESI+): m/z 425.1 [80% (M+H)⁺], 213.1 [100%, ((M+2H)²⁺/2)].

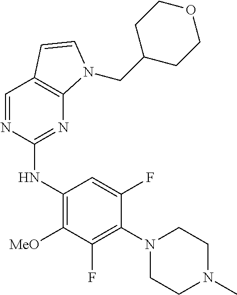 [SMILES: COc1c(Nc2ncc3ccn(CC4CCOCC4)c3n2)cc(F)c(N2CCN(C)CC2)c1F]

N-(3,5-Difluoro-2-methoxy-4-(4-methylpiperazin-1-yl)phenyl)-7-((tetrahydro-2H-pyran-4-yl)methyl)-7H-pyrrolo[2,3-d]pyrimidin-2-amine (SR8-140). SR8-140 was obtained as brown foam (0.042 g, 50% yield) from SR7-110 (0.050 g, 0.199 mmol) and YM3-023 (0.046 g, 0.179 mmol) by following the general procedure B. HPLC: >99% [t_{R}=12.1 min, gradient 5-95% MeOH and water (with 0.1% TFA), 20 min]. ¹H NMR (500 MHz, DMSO) δ 8.74 (s, 1H), 8.29 (dd, J=15.1, 2.0 Hz, 1H), 8.10 (d, J=1.2 Hz, 1H), 7.35 (d, J=3.5 Hz, 1H), 6.49 (d, J=3.5 Hz, 1H), 4.07 (d, J=7.2 Hz, 2H), 3.88 (s, 3H), 3.84 (ddd, J=11.4, 4.5, 1.9 Hz, 2H), 3.24 (td, J=11.6, 2.1 Hz, 2H), 3.09 (m, 4H), 2.46-2.41 (m, 4H), 2.23 (s, 3H), 2.23-2.13 (m, 1H), 1.48-1.39 (m, 2H), 1.34-1.25 (m, 2H); ¹⁹F NMR (471 MHz, DMSO) δ −125.23 (d, J=15.0 Hz), δ −138.08 (s); HRMS (ESI+): m/z calcd for C₂₄H₃₁F₂N₆O₂(M+H)⁺ 473.2471, found 473.2459 m/z calcd for C₂₄H₃₀F₂N₆O₂Na (M+Na)⁺ 495.2291, found 495.2272; HPLC-MS (ESI+): m/z 473.0 [100% (M+H)⁺], 237.1 [70%, ((M+2H)²⁺/2)].

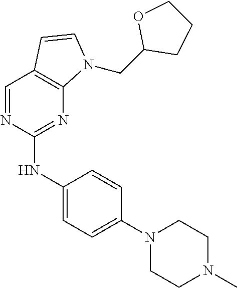 [SMILES: CN1CCN(c2ccc(Nc3ncc4ccn(CC5CCCO5)c4n3)cc2)CC1]

N-(4-(4-Methylpiperazin-1-yl)phenyl)-7-((tetrahydrofuran-2-yl)methyl)-7H-pyrrolo[2,3-d]pyrimidin-2-amine (SR7-131). SR7-131 was obtained as a foam (0.046 g, 56% yield) from SR7-126 (0.050 g, 0.210 mmol) and 4(4-methylpiperazine-1-yl)aniline (0.036 g, 0.189 mmol) by following the general procedure A. HPLC: >99% [t_{R}=12.8 min, gradient 5-95% MeOH and water (with 0.1% TFA), 20 min]. ¹H NMR (500 MHz, DMSO) δ 9.10 (s, 1H), 8.62 (s, 1H), 7.69 (d, J=9.1 Hz, 2H), 7.19 (d, J=3.6 Hz, 1H), 6.87 (d, J=9.2 Hz, 2H), 6.38 (d, J=3.7 Hz, 1H), 4.27-4.17 (m, 1H), 4.15 (m, 2H), 3.78 (m, 1H), 3.63 (m, 1H), 3.04 (m, 4H), 2.45 (t, J=5.0 Hz, 4H), 2.20 (s, 3H), 1.91 (m, 1H), 1.84-1.70 (m, 2H), 1.66-1.55 (m, 1H); ¹³C NMR (126 MHz, DMSO) δ 156.6, 152.2, 150.7, 145.9, 134.3, 127.5, 119.6, 116.5, 112.0, 99.7, 77.5, 67.8, 55.3, 49.6, 47.5, 46.3, 28.9, 25.5. HRMS (ESI+): m/z calcd for C₂₂H₂₉N₆O (M+H)⁺ 393.2397, found 393.2393 m/z calcd for C₂₂H₂₈N₆ONa (M+Na)⁺ 415.2217, found 415.2237; HPLC-MS (ESI+): m/z 393.2 [40% (M+H)⁺], 197.2 [100 ((M+2H)²⁺/2)].

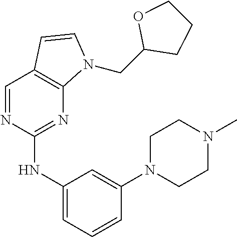 [SMILES: CN1CCN(c2cccc(Nc3ncc4ccn(CC5CCCO5)c4n3)c2)CC1]

N-(3-(4-Methylpiperazin-1-yl)phenyl)-7-((tetrahydrofuran-2-yl)methyl)-7H-pyrrolo[2,3-d]pyrimidin-2-amine (SR7-132). SR7-132 was obtained as a foam (0.040 g, 54% yield) from SR7-126 (0.050 g, 0.210 mmol) and 3-(4-methylpiperazine-1-yl)aniline (0.036 g, 0.189 mmol) by following the general procedure A. HPLC: >99% [t_{R}=13.4 min, gradient 5-95% MeOH and water (with 0.1% TFA), 20 min]. ¹H NMR (500 MHz, DMSO) δ 9.25 (s, 1H), 8.68 (s, 1H), 7.83 (t, J=2.1 Hz, 1H), 7.25 (d, J=3.6 Hz, 1H), 7.13 (d, J=8.5 Hz, 1H), 7.08 (t, J=8.0 Hz, 1H), 6.50 (ddd, J=8.0, 2.5, 1.1 Hz, 1H), 6.42 (d, J=3.6 Hz, 1H), 4.30-4.13 (m, 3H), 3.79 (ddd, J=8.2, 7.2, 6.0 Hz, 1H), 3.64 (dt, J=8.2, 7.0 Hz, 1H), 3.15 (t, J=5.0 Hz, 4H), 2.48 (t, J=5.0 Hz, 4H), 2.24 (s, 3H), 1.95-1.86 (m, 1H), 1.84-1.70 (m, 2H), 1.60 (m, 1H); ¹³C NMR (126 MHz, DMSO) δ 155.8, 151.9, 151.8, 150.9, 142.5, 129.1, 127.8, 112.2, 109.7, 108.6, 105.6, 99.8, 77.4, 67.8, 55.2, 48.9, 47.7, 46.3, 29.0, 25.5. HRMS (ESI+): m/z calcd for C₂₂H₂₉N₆O (M+H)⁺ 393.2397, found 393.2395 m/z calcd for C₂₂H₂₈N₆ONa (M+Na)⁺ 415.2217, found 415.2221; HPLC-MS (ESI+): m/z 393.2 [90% (M+H)⁺], 197.2 [100%, ((M+2H)²⁺/2)].

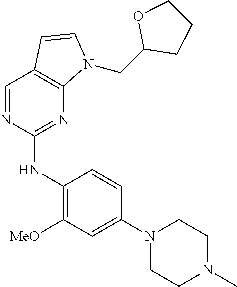 [SMILES: COc1cc(N2CCN(C)CC2)ccc1Nc1ncc2ccn(CC3CCCO3)c2n1]

N-(2-Methoxy-4-(4-methylpiperazin-1-yl)phenyl)-7-((tetrahydrofuran-2-yl)methyl)-7H-pyrrolo[2,3-d]pyrimidin-2-amine (SR7-133). SR7-133 was obtained as a foam (0.042 g, 53% yield) from SR7-126 (0.050 g, 0.210 mmol) and 2-methoxy-4-(4-methylpiperazine-1-yl)aniline (0.042 g, 0.189 mmol) by following the general procedure A. HPLC: >99% [t_{R}=13.4 min, gradient 5-95% MeOH and water (with 0.1% TFA), 20 min]. ¹H NMR (500 MHz, DMSO) δ 8.62 (s, 1H), 8.18 (d, J=8.8 Hz, 1H), 7.56 (s, 1H), 7.22 (d, J=3.6 Hz, 1H), 6.66 (d, J=2.6 Hz, 1H), 6.51 (dd, J=8.9, 2.6 Hz, 1H), 6.40 (d, J=3.6 Hz, 1H), 4.21 (m, 1H), 4.16 (dd, J=5.5, 1.5 Hz, 2H), 3.86 (s, 3H), 3.78 (ddd, J=8.2, 7.1, 6.1 Hz, 1H), 3.63 (dt, J=8.2, 6.8 Hz, 1H), 3.11 (t, J=5.0 Hz, 4H), 2.48 (m, 4H), 2.24 (s, 3H), 1.96-1.86 (m, 1H), 1.83-1.72 (m, 2H), 1.66-1.55 (m, 1H); ¹³C NMR (126 MHz, DMSO) δ 156.4, 152.3, 150.8, 149.7, 147.1, 127.8, 122.5, 120.1, 112.3, 107.4, 100.7, 99.7, 77.5, 67.8, 56.2, 55.4, 55.2, 49.5, 47.5, 46.2, 28.9, 25.5. HRMS (ESI+): m/z calcd for C₂₃H₃₁N₆O₂ (M+H)⁺ 423.2503, found 423.2496 m/z calcd for C₂₃H₃₀N₆O₂Na (M+Na)⁺ 445.2322, found 445.2311; HPLC-MS (ESI+): m/z 423.2 [40% (M+H)⁺], 212.2 [100%, ((M+2H)²⁺/2)].

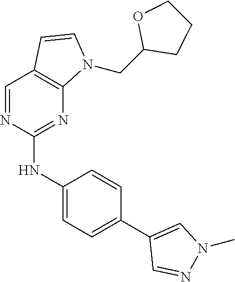 [SMILES: Cn1cc(-c2ccc(Nc3ncc4ccn(CC5CCCO5)c4n3)cc2)cn1]

N-(4-(1-Methyl-1H-pyrazol-4-yl)phenyl)-7-((tetrahydrofuran-2-yl)methyl)-7H-pyrrolo[2,3-d]pyrimidin-2-amine (SR7-159). SR7-159 was obtained as a yellow solid (0.023 g, 29% yield) from SR7-126 (0.050 g, 0.210 mmol) and 4-(1-methyl-1H-pyrazol-4-yl)aniline (0.036 g, 0.210 mmol) by following the general procedure B. HPLC: >99% [t_{R}=16.5 min, gradient 5-95% MeOH and water (with 0.1% TFA), 20 min]. ¹H NMR (500 MHz, DMSO) δ 9.40 (s, 1H), 8.69 (s, 1H), 8.03 (d, J=0.9 Hz, 1H), 7.88-7.82 (m, 2H), 7.78 (d, J=0.9 Hz, 1H), 7.47 (m, 2H), 7.26 (dd, J=3.4, 0.8 Hz, 1H), 6.43 (dd, J=3.5, 0.8 Hz, 1H), 4.25 (m, 1H), 4.21 (dd, J=5.5, 2.8 Hz, 2H), 3.86 (s, 3H), 3.80 (ddd, J=8.3, 7.0, 6.2 Hz, 1H), 3.65 (dt, J=8.3, 6.9 Hz, 1H), 1.93 (m, 1H), 1.80 (m, 2H), 1.68-1.57 (m, 1H); ¹³C NMR (126 MHz, DMSO) δ 156.2, 152.1, 150.8, 140.0, 136.0, 128.0, 127.4, 125.6, 125.4, 122.6, 118.8, 112.4, 99.8, 77.6, 67.8, 47.6, 39.1, 28.9, 25.5. HRMS (ESI+): m/z calcd for C₂₁H₂₂N₆O (M+H)⁺ 375.1928, found 375.1926 m/z calcd for C₂₁H₂₃N₆ONa (M+Na)⁺ 397.1747, found 397.1760; HPLC-MS (ESI+): m/z 375.2 [100% (M+H)⁺].

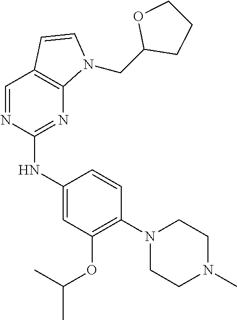 [SMILES: CC(C)Oc1cc(Nc2ncc3ccn(CC4CCCO4)c3n2)ccc1N1CCN(C)CC1]

N-(3-Isopropoxy-4-(4-methylpiperazin-1-yl)phenyl)-7-((tetrahydrofuran-2-yl)methyl)-7H-pyrrolo[2,3-d]pyrimidin-2-amine (SR7-166). SR7-166 was obtained as an off-white foam (0.064 g, 68% yield) from SR7-126 (0.050 g, 0.210 mmol) and 3-isopropoxy-4-(4-methylpiperazin-1-yl)aniline (0.047 g, 0.189 mmol) by following the general procedure B. HPLC: >99% [t_{R}=14.4 min, gradient 5-95% MeOH and water (with 0.1% TFA), 20 min]. ¹H NMR (500 MHz, DMSO) δ 9.20 (s, 1H), 8.65 (s, 1H), 7.75 (d, J=2.4 Hz, 1H), 7.26-7.20 (m, 2H), 6.81 (d, J=8.6 Hz, 1H), 6.41 (d, J=3.5 Hz, 1H), 4.60 (sep, J=6.1 Hz, 1H), 4.23 (m, 1H), 4.18 (m, 2H), 3.79 (ddd, J=8.2, 7.2, 6.0 Hz, 1H), 3.64 (m, 1H), 2.97 (bs, 4H), 2.55 (bs, 4H), 2.30 (s, 3H), 1.91 (m, 1H), 1.84-1.72 (m, 2H), 1.65-1.56 (m, 1H), 1.32 (d, J=6.1 Hz, 6H); ¹³C NMR (126 MHz, DMSO) δ 167.8, 156.4, 152.0, 150.9, 150.3, 137.2, 127.6, 118.8, 112.1, 111.2, 107.5, 99.8, 77.5, 70.1, 67.9, 55.4, 50.4, 47.6, 29.0, 25.5, 22.6, 22.6. HRMS (ESI+): m/z calcd for C₂₅H₃₅N₆O₂ (M+H)⁺ 451.2816, found 451.2812 m/z calcd for C₂₅H₃₄N₆O₂Na (M+Na)⁺ 473.2635, found 473.2625; HPLC-MS (ESI+): m/z 451.3 [40% (M+H)⁺], 226.3 [100%, ((M+2H)²⁺/2)].

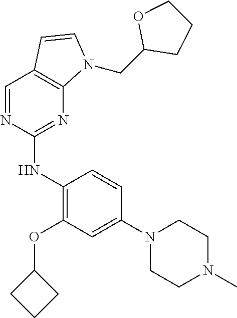 [SMILES: CN1CCN(c2ccc(Nc3ncc4ccn(CC5CCCO5)c4n3)c(OC3CCC3)c2)CC1]

N-(2-Cyclobutoxy-4-(4-methylpiperazin-1-yl)phenyl)-7-((tetrahydrofuran-2-yl)methyl)-7H-pyrrolo[2,3-d]pyrimidin-2-amine (SR7-169). SR7-169 was obtained as yellow foam (0.059 g, 61% yield) from SR7-126 (0.050 g, 0.210 mmol) and 2-cyclobutoxy-4-(4-methylpiperazin-1-yl)aniline (0.055 g, 0.210 mmol) by following the general procedure B. HPLC: >99% [t_{R}=14.6 min, gradient 5-95% MeOH and water (with 0.1% TFA), 20 min]. ¹H NMR (500 MHz, DMSO) δ 8.63 (s, 1H), 8.25 (d, J=8.7 Hz, 1H), 7.52 (s, 1H), 7.23 (d, J=3.5 Hz, 1H), 6.51 (dd, J=8.9, 2.6 Hz, 1H), 6.47 (d, J=2.6 Hz, 1H), 6.41 (d, J=3.6 Hz, 1H), 4.79 (p, J=7.1 Hz, 1H), 4.22 (m, 1H), 4.17 (dd, J=5.5, 2.0 Hz, 2H), 3.78 (ddd, J=8.2, 7.1, 6.1 Hz, 1H), 3.64 (dt, J=8.2, 6.9 Hz, 1H), 3.08 (m, 4H), 2.49-2.39 (m, 6H), 2.24 (s, 3H), 2.15-2.04 (m, 2H), 1.97-1.86 (m, 1H), 1.84-1.72 (m, 3H), 1.71-1.54 (m, 2H); ¹³C NMR (126 MHz, DMSO) δ 156.2, 152.2, 150.8, 147.0, 146.8, 127.8, 122.7, 119.8, 112.3, 107.7, 101.9, 99.8, 77.5, 71.6, 67.8, 55.2, 49.4, 47.5, 46.2, 30.8, 28.9, 25.5, 13.2. HRMS (ESI+): m/z calcd for C₂₆H₃₅N₆O₂ (M+H)⁺ 463.2816, found 463.2809; HPLC-MS (ESI+): m/z 463.3 [50% (M+H)⁺], 232.2 [100%, ((M+2H)²⁺/2)].

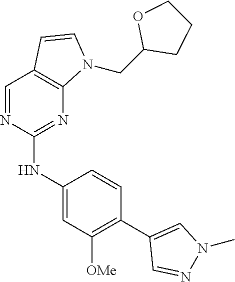 [SMILES: COc1cc(Nc2ncc3ccn(CC4CCCO4)c3n2)ccc1-c1cnn(C)c1]

N-(3-methoxy-4-(1-methyl-1H-pyrazol-4-yl)phenyl)-7-((tetrahydrofuran-2-yl)methyl)-7H-pyrrolo[2,3-d]pyrimidin-2-amine (SR7-172). SR7-172 was obtained as yellow foam (0.054 g, 64% yield) from SR7-126 (0.050 g, 0.210 mmol) and 3-methoxy-4-(1-methyl-1H-pyrazol-4-yl)aniline (0.043 g, 0.210 mmol) by following the general procedure B. HPLC: >99% [t_{R}=17.3 min, gradient 5-95% MeOH and water (with 0.1% TFA), 20 min]. ¹H NMR (500 MHz, DMSO) δ 9.47 (s, 1H), 8.70 (s, 1H), 8.05 (d, J=2.1 Hz, 1H), 8.01 (d, J=0.7 Hz, 1H), 7.82 (d, J=0.7 Hz, 1H), 7.46 (d, J=8.4 Hz, 1H), 7.31-7.24 (m, 2H), 6.44 (d, J=3.5 Hz, 1H), 4.34-4.18 (m, 3H), 3.92 (s, 3H), 3.86 (s, 3H), 3.79 (ddd, J=8.2, 7.2, 6.0 Hz, 1H), 3.64 (ddd, J=8.2, 7.2, 6.4 Hz, 1H), 1.94-1.86 (m, 1H), 1.85-1.71 (m, 2H), 1.66-1.53 (m, 1H); ¹³C NMR (126 MHz, DMSO) δ 156.1, 155.9, 151.8, 150.9, 141.1, 137.3, 129.1, 128.0, 127.1, 118.6, 113.7, 112.4, 110.8, 102.0, 99.8, 77.4, 67.9, 55.5, 47.8, 38.9, 28.9, 25.5. HRMS (ESI+): m/z calcd for C₂₂H₂₅N₆O₂ (M+H)⁺ 405.2034, found 405.2032 m/z calcd for C₂₂H₂₄N₆O₂Na (M+Na)⁺ 427.1853, found 427.1859; HPLC-MS (ESI+): m/z 405.2 [100% (M+H)⁺], 203.2 [10%, ((M+2H)²⁺/2)].

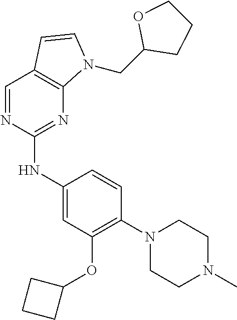 [SMILES: CN1CCN(c2ccc(Nc3ncc4ccn(CC5CCCO5)c4n3)cc2OC2CCC2)CC1]

N-(3-Cyclobutoxy-4-(4-methylpiperazin-1-yl)phenyl)-7-((tetrahydrofuran-2-yl)methyl)-7H-pyrrolo[2,3-d]pyrimidin-2-amine (SR8-036). SR8-036 was obtained as yellow foam (0.057 g, 65% yield) from SR7-126 (0.050 g, 0.210 mmol) and 3-cyclobutoxy-4-(4-methylpiperazin-1-yl)aniline (SR8-029) (0.049 g, 0.189 mmol) by following the general procedure B. HPLC: >99% [t_{R}=15.7 min, gradient 5-95% MeOH and water (with 0.1% TFA), 20 min]. ¹H NMR (500 MHz, DMSO) δ 9.20 (s, 1H), 8.65 (s, 1H), 7.48 (d, J=2.3 Hz, 1H), 7.33 (dd, J=8.6, 2.4 Hz, 1H), 7.23 (d, J=3.5 Hz, 1H), 6.80 (d, J=8.6 Hz, 1H), 6.41 (d, J=3.5 Hz, 1H), 4.67 (p, J=7.1 Hz, 1H), 4.29-4.16 (m, 3H), 3.80 (ddd, J=8.2, 7.1, 6.0 Hz, 1H), 3.65 (dt, J=8.1, 6.7 Hz, 1H), 2.97 (bs, 4H), 2.59 (bs, 4H), 2.50-2.42 (m, 1H), 2.32 (s, 3H), 2.14-2.02 (m, 2H), 1.97-1.86 (m, 1H), 1.87-1.73 (m, 3H), 1.73-1.66 (m, 1H), 1.66-1.57 (m, 1H); ¹³C NMR (126 MHz, DMSO) δ 156.4, 152.1, 150.8, 150.1, 137.3, 127.7, 118.6, 112.2, 110.6, 105.2, 99.8, 77.5, 71.3, 67.9, 55.2, 50.4, 47.6, 30.9, 29.0, 25.6. HRMS (ESI+): m/z calcd for C₂₆H₃₅N₆O₂ (M+H)⁺ 463.2816, found 463.2812, m/z calcd for C₂₆H₃₄N₆O₂Na (M+Na)⁺ 485.2635, found 485.2638; HPLC-MS (ESI+): m/z 463.3 [30% (M+H)⁺], 232.2 [100%, ((M+2H)²⁺/2)].

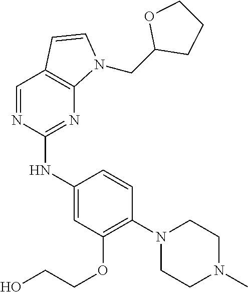 [SMILES: CN1CCN(c2ccc(Nc3ncc4ccn(CC5CCCO5)c4n3)cc2OCCO)CC1]

2-(2-(4-Methylpiperazin-1-yl)-5-((7-((tetrahydrofuran-2-yl)methyl)-7H-pyrrolo[2,3-d]pyrimidin-2-yl)amino)phenoxy)ethan-1-ol (SR8-042). SR8-042 was obtained as yellow foam (0.054 g, 63% yield) from SR7-126 (0.050 g, 0.210 mmol) and 2-(5-amino-2-(4-methylpiperazin-1-yl)phenoxy)ethan-1-ol (SR8-040) (0.048 g, 0.189 mmol) by following the general procedure B. HPLC: >99% [t_{R}=13.4 min, gradient 5-95% MeOH and water (with 0.1% TFA), 20 min]. ¹H NMR (500 MHz, DMSO) δ 9.21 (s, 1H), 8.65 (s, 1H), 7.80 (d, J=2.4 Hz, 1H), 7.27-7.17 (m, 2H), 6.81 (d, J=8.6 Hz, 1H), 6.41 (d, J=3.5 Hz, 1H), 4.85 (t, J=5.5 Hz, 1H), 4.24 (m, 1H), 4.20 (m, 1H), 4.03 (t, J=5.2 Hz, 2H), 3.82-3.71 (m, 3H), 3.63 (m, 1H), 2.96 (bs, 4H), 2.50-2.39 (bs, 4H), 2.24 (s, 3H), 1.98-1.87 (m, 1H), 1.84-1.70 (m, 2H), 1.67-1.52 (m, 1H); ¹³C NMR (126 MHz, DMSO) δ 156.3, 151.9, 151.7, 150.9, 137.3, 127.7, 118.5, 112.1, 111.0, 105.5, 99.7, 77.5, 70.6, 67.8, 60.3, 55.5, 50.7, 47.7, 46.3, 28.9, 25.5. HRMS (ESI+): m/z calcd for C₂₄H₃₃N₆O₃ (M+H)⁺ 453.2609, found 453.2613; HPLC-MS (ESI+): m/z 453.3 [40% (M+H)⁺], 227.2 [100%, ((M+2H)²⁺/2)].

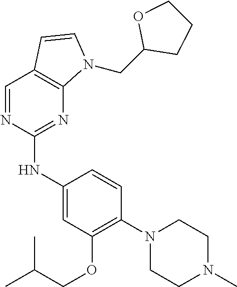 [SMILES: CC(C)COc1cc(Nc2ncc3ccn(CC4CCCO4)c3n2)ccc1N1CCN(C)CC1]

N-(3-isobutoxy-4-(4-methylpiperazin-1-yl)phenyl)-7-((tetrahydrofuran-2-yl)methyl)-7H-pyrrolo[2,3-d]pyrimidin-2-amine (SR8-054). SR8-054 was obtained as yellow foam (0.051 g, 58% yield) from SR7-126 (0.050 g, 0.210 mmol) and 3-isobutoxy-4-(4-methylpiperazin-1-yl)aniline (SR8-039) (0.050 g, 0.189 mmol) by following the general procedure B. HPLC: >99% [t_{R}=16.4 min, gradient 5-95% MeOH and water (with 0.1% TFA), 20 min]. ¹H NMR (600 MHz, DMSO) δ 9.20 (s, 1H), 8.65 (s, 1H), 7.80 (d, J=2.3 Hz, 1H), 7.22 (d, J=3.5 Hz, 1H), 7.19 (dd, J=8.6, 2.3 Hz, 1H), 6.79 (d, J=8.6 Hz, 1H), 6.41 (d, J=3.5 Hz, 1H), 4.28-4.12 (m, 3H), 3.77 (m, 3H), 3.63 (m, 1H), 2.96 (s, 4H), 2.51 (bs, 4H), 2.28 (s, 3H), 2.08 (m, 1H), 1.98-1.84 (m, 1H), 1.84-1.68 (m, 2H), 1.59 (m, 1H), 1.06 (d, J=6.7 Hz, 6H). HRMS (ESI+): m/z calcd for C₂₆H₃₇N₆O₂ (M+H)⁺ 465.2973, found 465.2969, m/z calcd for C₂₆H₃₆N₆O₂Na (M+Na)⁺ 487.2792, found 487.2749; HPLC-MS (ESI+): m/z 465.3 [30% (M+H)⁺], 233.2 [100%, ((M+2H)²⁺/2)].

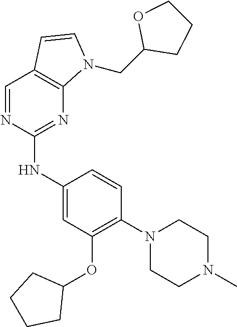 [SMILES: CN1CCN(c2ccc(Nc3ncc4ccn(CC5CCCO5)c4n3)cc2OC2CCCC2)CC1]

N-(3-(cyclopentyloxy)-4-(4-methylpiperazin-1-yl)phenyl)-7-((tetrahydrofuran-2-yl)methyl)-7H-pyrrolo[2,3-d]pyrimidin-2-amine (SR8-062). SR8-062 was obtained as yellow foam (0.058 g, 63% yield) from SR7-126 (0.050 g, 0.210 mmol) and 3-(cyclopentyloxy)-4-(4-methylpiperazin-1-yl)aniline (SR8-059) (0.052 g, 0.189 mmol) by following the general procedure B. HPLC: >99% [t_{R}=15.1 min, gradient 5-95% MeOH and water (with 0.1% TFA), 20 min]. ¹H NMR (600 MHz, DMSO) δ 9.19 (s, 1H), 8.65 (s, 1H), 7.70 (d, J=2.3 Hz, 1H), 7.23 (m, 2H), 6.78 (d, J=8.5 Hz, 1H), 6.41 (d, J=3.5 Hz, 1H), 4.84 (m, 1H), 4.26-4.16 (m, 3H), 3.78 (dd, J=7.6, 6.0 Hz, 1H), 3.63 (m, 1H), 2.93 (bs, 4H), 2.51 (bs, J=1.9 Hz, 4H), 2.25 (s, 3H), 1.96-1.85 (m, 3H), 1.84-1.72 (m, 6H), 1.68-1.51 (m, 3H). HRMS (ESI+): m/z calcd for C₂₇H₃₇N₆O₂ (M+H)⁺ 477.2973, found 477.2967, m/z calcd for C₂₇H₃₆N₆O₂Na (M+Na)⁺ 499.2792, found 499.2801; HPLC-MS (ESI+): m/z 477.3 [30% (M+H)⁺], 239.2 [100%, ((M+2H)²⁺/2)].

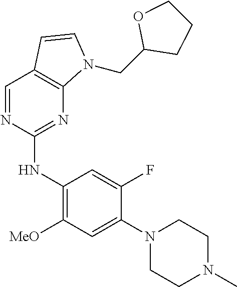 [SMILES: COc1cc(N2CCN(C)CC2)c(F)cc1Nc1ncc2ccn(CC3CCCO3)c2n1]

N-(5-Fluoro-2-methoxy-4-(4-methylpiperazin-1-yl)phenyl)-7-((tetrahydrofuran-2-yl)methyl)-7H-pyrrolo[2,3-d]pyrimidin-2-amine (SR8-096). SR8-096 was obtained as yellow foam (0.043 g, 52% yield) from SR7-126 (0.050 g, 0.210 mmol) and 3-fluoro-6-methoxy-4-(4-methylpiperazin-1-yl)aniline (YM2-091) (0.045 g, 0.189 mmol) by following the general procedure B. HPLC: >99% [t_{R}=14.4 min, gradient 5-95% MeOH and water (with 0.1% TFA), 20 min]. ¹H NMR (500 MHz, DMSO) δ 8.69 (s, 1H), 8.36 (d, J=15.2 Hz, 1H), 7.68 (d, J=1.3 Hz, 1H), 7.29 (d, J=3.6 Hz, 1H), 6.71 (d, J=8.2 Hz, 1H), 6.45 (d, J=3.5 Hz, 1H), 4.25 (m, 1H), 4.18 (d, J=5.9 Hz, 2H), 3.90 (s, 3H), 3.79 (dt, J=8.2, 6.6 Hz, 1H), 3.64 (dt, J=8.2, 6.9 Hz, 1H), 3.02 (t, J=4.9 Hz, 4H), 2.50 (bs, 4H), 2.25 (s, 3H), 1.99-1.88 (m, 1H), 1.84-1.75 (m, 2H), 1.70-1.58 (m, 1H); ¹⁹F NMR (471 MHz, DMSO) δ −131.16 (dd, J=15.1, 8.2 Hz); HRMS (ESI+): m/z calcd for C₂₃H₃₀FN₆O₂(M+H)⁺ 441.2409, found 441.2405, m/z calcd for C₂₃H₂₉FN₆O₂Na (M+Na)⁺ 463.2228, found 463.2241; HPLC-MS (ESI+): m/z 441.0 [40% (M+H)⁺], 221.1 [100%, ((M+2H)²⁺/2)].

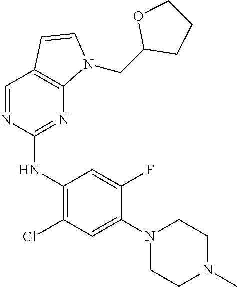 [SMILES: CN1CCN(c2cc(Cl)c(Nc3ncc4ccn(CC5CCCO5)c4n3)cc2F)CC1]

N-(2-Chloro-5-fluoro-4-(4-methylpiperazin-1-yl)phenyl)-7-((tetrahydrofuran-2-yl)methyl)-7H-pyrrolo[2,3-d]pyrimidin-2-amine (SR8-121). SR8-121 was obtained as yellow foam (0.046 g, 58% yield) from SR7-126 (0.050 g, 0.210 mmol) and SR8-120 (0.046 g, 0.189 mmol) by following the general procedure B. HPLC: >98% [t_{R}=15.1 min, gradient 5-95% MeOH and water (with 0.1% TFA), 20 min]. ¹H NMR (500 MHz, DMSO) δ 8.69 (s, 1H), 8.16-8.11 (m, 2H), 7.30 (d, J=3.6 Hz, 1H), 7.10 (d, J=8.8 Hz, 1H), 6.46 (d, J=3.6 Hz, 1H), 4.25-4.17 (m, 1H), 4.15 (d, J=5.7 Hz, 2H), 3.78 (dt, J=8.3, 6.6 Hz, 1H), 3.63 (dt, J=8.2, 6.9 Hz, 1H), 3.00 (t, J=4.9 Hz, 4H), 2.49-2.45 (m, 4H), 2.23 (s, 3H), 1.96-1.85 (m, 1H), 1.84-1.72 (m, 2H), 1.68-1.53 (m, 1H); ¹⁹F NMR (471 MHz, DMSO) δ −122.74 (dd, J=15.1, 8.9 Hz); HRMS (ESI+): m/z calcd for C₂₂H₂₇ClFN₆O (M+H)⁺ 445.1913, found 445.1809, m/z calcd for C₂₂H₂₆ClFN₆ONa (M+Na)⁺ 467.1733, found 467.1728; HPLC-MS (ESI+): m/z 445.1 [90% (M+H)⁺], 223.2 [100%, ((M+2H)²⁺/2)].

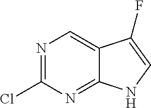 [SMILES: Fc1c[nH]c2nc(Cl)ncc12]

2-Chloro-5-fluoro-7H-pyrrolo[2,3-d]pyrimidine (SR7-136). SR7-136 was obtained as a brown solid (0.304 g, 27%) from the reaction of 2-chloropyrrolopyrimidine with Selectfluor™ according to a reported procedure (see WO2018200425). ¹H NMR (500 MHz, DMSO-d6) δ 12.30 (s, 1H), 9.04 (s, 1H), 7.64 (t, J=2.5 Hz, 1H); ¹⁹F NMR (471 MHz, DMSO-d6) δ −170.69 (d, J=1.7 Hz).

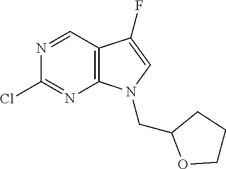 [SMILES: Fc1cn(CC2CCCO2)c2nc(Cl)ncc12]

2-Chloro-5-fluoro-7-((tetrahydrofuran-2-yl)methyl)-7H-pyrrolo[2,3-d]pyrimidine (SR7-140). SR7-140 (0.250 g, 1.462 mmol) was dissolved in dry DMF (5 mL) and K₂CO₃ (0.606 g, 4.386 mmol) and (tetrahydrofuran-2-yl)methyl 4-methylbenzenesulfonate (SR7-120) (0.412 g, 1.608 mmol) were added. The mixture was heated at 85° C. in a sealed vial for 34 h and allowed cool to rt. The resulting suspension was then diluted with EtOAc (40 mL) and washed with water (2×25 mL) followed by brine (1×25 mL). ¹H NMR (500 MHz, DMSO) δ 9.05 (d, J=1.1 Hz, 1H), 7.70 (d, J=2.3 Hz, 1H), 4.26-4.13 (m, 2H), 4.04-3.84 (m, 1H), 3.79-3.68 (m, 1H), 3.63 (m, 1H), 1.99-1.88 (m, 1H), 1.87-1.73 (m, 2H), 1.55 (m, 1H). ¹⁹F NMR (471 MHz, DMSO) δ −170.74.

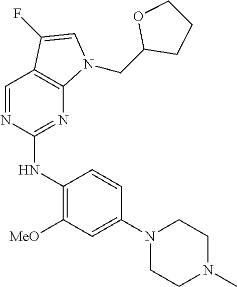 [SMILES: COc1cc(N2CCN(C)CC2)ccc1Nc1ncc2c(F)cn(CC3CCCO3)c2n1]

5-Fluoro-N-(2-methoxy-4-(4-methylpiperazin-1-yl)phenyl)-7-((tetrahydrofuran-2-yl)methyl)-7H-pyrrolo[2,3-d]pyrimidin-2-amine (SR7-143). SR7-143 was obtained as a foam (0.018 mg, 23% yield) from SR7-140 (0.050 g, 0.195 mmol) and 2-methoxy-4-(4-methylpiperazine-1-yl)aniline (0.038 g, 0.175 mmol) by following the general procedure A. HPLC: >99% [t_{R}=14.1 min, gradient 5-95% MeOH and water (with 0.1% TFA), 20 min]. ¹H NMR (500 MHz, DMSO) δ 8.69 (d, J=1.0 Hz, 1H), 8.02 (d, J=8.7 Hz, 1H), 7.77 (s, 1H), 7.16 (d, J=2.2 Hz, 1H), 6.65 (d, J=2.6 Hz, 1H), 6.51 (dd, J=8.8, 2.6 Hz, 1H), 4.19 (m, 1H), 4.13-4.03 (m, 2H), 3.84 (s, 3H), 3.77 (dt, J=8.1, 6.7 Hz, 1H), 3.63 (dt, J=8.2, 6.8 Hz, 1H), 3.12 (t, J=5.0 Hz, 4H), 2.47 (td, J=4.3, 2.3 Hz, 4H), 2.24 (s, 3H), 1.98-1.84 (m, 1H), 1.84-1.71 (m, 2H), 1.66-1.52 (m, 1H); ¹⁹F NMR (471 MHz, DMSO) δ −172.17; HRMS (ESI+): m/z calcd for C₂₃H₃₀FN₆O₂(M+H)⁺ 441.2409, found 441.2411, m/z calcd for C₂₃H₂₉FN₆O₂Na (M+Na)⁺ 463.2228, found 463.2248; HPLC-MS (ESI+): m/z 441.2 [100% (M+H)⁺], 221.2 [100%, ((M+2H)²⁺/2)].

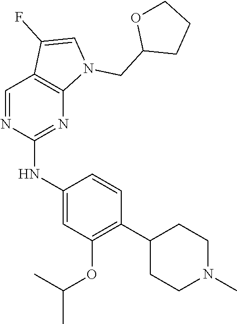 [SMILES: CC(C)Oc1cc(Nc2ncc3c(F)cn(CC4CCCO4)c3n2)ccc1C1CCN(C)CC1]

5-Fluoro-N-(3-isopropoxy-4-(4-methylpiperazin-1-yl)phenyl)-7-((tetrahydrofuran-2-yl)methyl)-7H-pyrrolo[2,3-d]pyrimidin-2-amine (SR7-144). SR7-144 was obtained as a yellow foam (0.021 g, 26% yield) from SR7-126 (0.050 g, 0.210 mmol) and 3-isopropoxy-4-(1-methylpiperadin-4-yl)aniline (0.043 g, 0.175 mmol) by following the general procedure A. HPLC: >98% [t_{R}=17.1 min, gradient 5-95% MeOH and water (with 0.1% TFA), 20 min]. ¹H NMR (500 MHz, DMSO) δ 9.49 (s, 1H), 8.76 (s, 1H), 7.80 (d, J=2.1 Hz, 1H), 7.20 (dd, J=9.4, 2.1 Hz, 2H), 7.03 (d, J=8.4 Hz, 1H), 4.63-4.51 (m, 1H), 4.26-4.17 (m, 1H), 4.16-4.05 (m, 2H), 3.79 (ddd, J=8.3, 7.1, 6.0 Hz, 1H), 3.64 (dt, J=8.2, 6.8 Hz, 1H), 3.30 (s, 2H), 2.92 (d, J=11.0 Hz, 2H), 2.76 (tt, J=9.6, 4.7 Hz, 1H), 2.25 (s, 3H), 2.13-1.97 (m, 1H), 1.95-1.86 (m, 1H), 1.84-1.72 (m, 2H), 1.72-1.63 (m, 3H), 1.64-1.47 (m, 1H), 1.33 (d, J=6.0 Hz, 6H); ¹⁹F NMR (471 MHz, DMSO) δ −172.14. HRMS (ESI+): m/z calcd for C₂₆H₃₅FN₅O₂(M+H)⁺ 468.2769, found 468.2763 m/z calcd for C₂₆H₃₄FN₅O₂Na (M+Na)⁺ 490.2589, found 490.2591; HPLC-MS (ESI+): m/z 468.3 [100% (M+H)⁺], 234.7 [100%, ((M+2H)²⁺/2)].

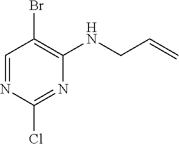

N-allyl-5-bromo-2-chloropyrimidin-4-amine (SR7-139). 5-bromo-2,4-dichloropyrimidine (2.00 g, 8.776 mmol) was dissolved in ethanol (40 mL) and DIPEA (2.293 mL, 13.164 mmol) and allyl bromide (0.789 mL, 10.532 mmol) were added. The mixture was stirred for 20 h at 50° C. and concentrated under reduced pressure. The resulting residue was dissolved in EtOAc (30 mL) and washed with sat. NH₄Cl (25 mL×1) and brine (25 mL×1). Purification by column chromatography (0-50% gradient elution, EtOAc:hexane) afforded the titled product as a solid (1.712 g, 79% yield). (see WO2007140222)¹H NMR (500 MHz, CDCl₃) δ 8.14 (s, 1H), 5.94 (ddt, J=17.1, 10.3, 5.7 Hz, 1H), 5.58 (s, 1H), 5.29 (m, 1H), 5.24 (dq, J=10.2, 1.3 Hz, 1H), 4.16 (tt, J=5.7, 1.5 Hz, 2H).

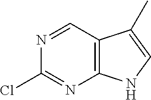

2-Chloro-5-methyl-7H-pyrrolo[2,3-d]pyrimidine (SR7-145).⁶ SR7-139 (2.00 g, 8.048 mmol) was dissolved in dry DMF (30 mL) and trimethylamine (2.243 mL, 16.096 mmol) and P(O-Tolyl)₃ were added. The mixture was purged with Ar for 10 min and Pd(OAc)₂ (0.181 g, 0.805 mmol) added. The sealed flask was stirred at 100° C. for 24 h and allowed reach to rt. The mixture was filtered through Celite and the filtrate was diluted with EtOAc (30 mL) and washed with sat. NH₄Cl (25 mL×1) and brine (25 mL×1) then concentrated under reduced pressure. Purification by column chromatography (0-50% gradient elution, EtOAc:hexane) afforded the titled product as a solid (0.942 g, 70% yield). ¹H NMR (500 MHz, DMSO) δ 12.01 (s, 1H), 8.89 (s, 1H), 7.35 (t, J=1.3 Hz, 1H), 2.29 (d, J=1.2 Hz, 3H).

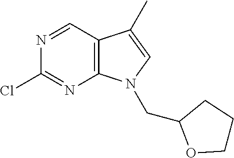 [SMILES: Cc1cn(CC2CCCO2)c2nc(Cl)ncc12]

2-Chloro-5-methyl-7-((tetrahydrofuran-2-yl)methyl)-7H-pyrrolo[2,3-d]pyrimidine (SR7-165). SR7-165 was obtained as a gum (0.550 g, 74% yield) from SR7-145 (0.500 g, 2.983 mmol) and tertrahydrofurfural bromide (0.492 g, 2.983 mmol) by following general procedure 1. ¹H NMR (500 MHz, DMSO) δ 8.90 (s, 1H), 7.40 (d, J=1.3 Hz, 1H), 4.22 (m, 1H), 4.16 (m, 2H), 3.77 (dt, J=8.3, 6.8 Hz, 1H), 3.63 (dt, J=8.2, 6.8 Hz, 1H), 2.30 (d, J=1.1 Hz, 3H), 2.01-1.89 (m, 1H), 1.83-1.73 (m, 2H), 1.55 (m, 1H).

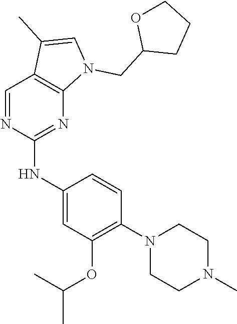 [SMILES: Cc1cn(CC2CCCO2)c2nc(Nc3ccc(N4CCN(C)CC4)c(OC(C)C)c3)ncc12]

N-(3-Isopropoxy-4-(4-methylpiperazin-1-yl)phenyl)-5-methyl-7-((tetrahydrofuran-2-yl)methyl)-7H-pyrrolo[2,3-d]pyrimidin-2-amine (SR7-175). SR7-175 was obtained as a yellow foam (0.054 g, 65% yield) from SR7-165 (0.050 g, 0.198 mmol) and 3-isopropoxy-4-(4-methylpiperazin-1-yl)aniline (0.044 g, 0.178 mmol) by following the general procedure B. HPLC: >99% [t_{R}=14.6 min, gradient 5-95% MeOH and water (with 0.1% TFA), 20 min]. ¹H NMR (500 MHz, DMSO) δ 9.16 (s, 1H), 8.62 (s, 1H), 7.75 (d, J=2.4 Hz, 1H), 7.22 (dd, J=8.6, 2.4 Hz, 1H), 6.96 (s, 1H), 6.80 (d, J=8.7 Hz, 1H), 4.59 (sep, J=6.1 Hz, 1H), 4.19 (m, 1H), 4.11 (d, J=5.7 Hz, 2H), 3.79 (dt, J=8.2, 6.6 Hz, 1H), 3.64 (dt, J=8.2, 6.9 Hz, 1H), 2.94 (s, 4H), 2.47 (s, 4H), 2.28-2.19 (m, 6H (two Me groups)), 1.95-1.85 (m, 1H), 1.85-1.74 (m, 2H), 1.64-1.51 (m, 1H), 1.31 (d, J=6.0 Hz, 6H); ¹³C NMR (126 MHz, DMSO) δ 156.4, 152.1, 150.3, 149.5, 137.2, 136.5, 124.5, 118.7, 112.8, 111.2, 109.1, 107.6, 77.6, 70.1, 67.8, 55.6, 50.7, 47.3, 46.3, 29.0, 25.5, 22.6, 10.0. HRMS (ESI+): m/z calcd for C₂₃H₃₇N₆O₂ (M+H)⁺ 465.2973, found 465.64 m/z calcd for C₂₃H₃₆N₆O₂Na (M+Na)⁺ 487.2792, found 487.2779; HPLC-MS (ESI+): m/z 465.3 [20% (M+H)⁺], 233.2 [100%, ((M+2H)²⁺/2)].

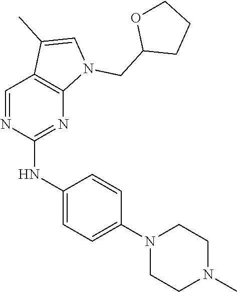 [SMILES: Cc1cn(CC2CCCO2)c2nc(Nc3ccc(N4CCN(C)CC4)cc3)ncc12]

5-Methyl-N-(4-(4-methylpiperazin-1-yl)phenyl)-7-((tetrahydrofuran-2-yl)methyl)-7H-pyrrolo[2,3-d]pyrimidin-2-amine (SR7-177). SR7-177 was obtained as a white foam (0.053 g, 73% yield) from SR7-165 (0.050 g, 0.198 mmol) and 4(4-methylpiperazine-1-yl)aniline (0.034 g, 0.178 mmol) by following the general procedure B. HPLC: >99% [t_{R}=12.7 min, gradient 5-95% MeOH and water (with 0.1% TFA), 20 min]. ¹H NMR (500 MHz, DMSO) δ 9.07 (s, 1H), 8.58 (s, 1H), 7.69 (d, J=9.1 Hz, 1H), 6.93 (d, J=1.2 Hz, 1H), 6.87 (d, J=9.1 Hz, 2H), 4.18 (m, 1H), 4.08 (d, J=5.6 Hz, 2H), 3.78 (dt, J=8.2, 6.7 Hz, 1H), 3.63 (dt, J=8.2, 6.8 Hz, 1H), 3.04 (t, J=4.9 Hz, 4H), 2.46 (t, J=4.9 Hz, 4H), 2.21 (m, 6H (two Me groups)), 1.90 (m, 1H), 1.79 (m, 2H), 1.65-1.54 (m, 1H); ¹³C NMR (126 MHz, DMSO) δ 156.6, 152.3, 149.4, 145.8, 134.4, 124.4, 119.6, 116.5, 112.7, 109.0, 77.6, 67.8, 55.2, 49.6, 47.2, 46.3, 28.9, 25.5, 10.0. HRMS (ESI+): m/z calcd for C₂₃H₃₁N₆O (M+H)⁺ 407.2554, found 407.2548 m/z calcd for C₂₃H₃₀N₆ONa (M+Na)⁺ 429.2373, found 423.2369; HPLC-MS (ESI+): m/z 407.2 [40% (M+H)⁺], 204.2 [100%, ((M+2H)²⁺/2)].

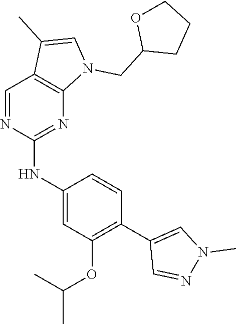 [SMILES: Cc1cn(CC2CCCO2)c2nc(Nc3ccc(-c4cnn(C)c4)c(OC(C)C)c3)ncc12]

N-(3-isoPropoxy-4-(1-methyl-1H-pyrazol-4-yl)phenyl)-5-methyl-7-((tetrahydrofuran-2-yl)methyl)-7H-pyrrolo[2,3-d]pyrimidin-2-amine (SR7-179). SR7-179 was obtained as a white foam (0.041 g, 52% yield) from SR7-165 (0.050 g, 0.198 mmol) and 3-isopropoxy-4-(1-methyl-1H-pyrazol-4-yl)aniline (0.041 g, 0.178 mmol) by following the general procedure B. HPLC: >99% [t_{R}=17.6 min, gradient 5-95% MeOH and water (with 0.1% TFA), 20 min]. ¹H NMR (500 MHz, DMSO) δ 9.40 (s, 1H), 8.67 (s, 1H), 8.00 (d, J=2.1 Hz, 1H), 7.98 (s, 1H), 7.83 (s, 1H), 7.43 (d, J=8.4 Hz, 1H), 7.24 (dd, J=8.5, 2.1 Hz, 1H), 7.01 (s, 1H), 4.69 (sep, J=6.0 Hz, 1H), 4.20 (m, 1H), 4.15 (dd, J=5.6, 2.0 Hz, 2H), 3.86 (s, 3H), 3.80 (ddd, J=8.2, 7.0, 6.2 Hz, 1H), 3.65 (dt, J=8.2, 6.8 Hz, 1H), 2.24 (d, J=1.3 Hz, 3H), 1.91 (m, 1H), 1.85-1.75 (m, 2H), 1.60 (m, 1H), 1.41 (d, J=6.0, 6H); ¹³C NMR (126 MHz, DMSO) δ 156.2, 153.9, 151.9, 149.6, 141.0, 137.4, 128.9, 127.4, 124.8, 118.9, 114.5, 113.0, 110.8, 109.2, 103.7, 77.5, 69.9, 67.9, 47.4, 39.0, 29.1, 25.5, 22.6, 22.5, 10.0. HRMS (ESI+): m/z calcd for C₂₅H₃₁N₆O₂ (M+H)⁺ 407.2554, found 407.2548 m/z calcd for C₂₅H₃₀N₆O₂Na (M+Na)⁺ 446.2503, found 447.2497; HPLC-MS (ESI+): m/z 447.2 [100% (M+H)⁺], 224.2 [20%, ((M+2H)²⁺/2)].

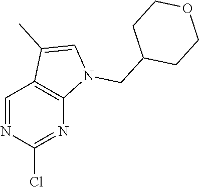 [SMILES: Cc1cn(CC2CCOCC2)c2nc(Cl)ncc12]

2-Chloro-5-methyl-7-((tetrahydro-2H-pyran-4-yl)methyl)-7H-pyrrolo[2,3-d]pyrimidine (SR7-183). SR7-183 was obtained as a gum (0.313 g, 79% yield) from SR7-145 (0.250 g, 1.497 mmol) 4-(bromomethyl)tetrahydro-2H-pyran (0.295 g, 1.640 mmol) by following general procedure 1. ¹H NMR (500 MHz, DMSO) δ 8.90 (s, OH), 7.42 (q, J=1.2 Hz, 1H), 4.05 (d, J=7.4 Hz, 2H), 3.81 (ddd, J=11.5, 4.3, 1.9 Hz, 2H), 3.22 (td, J=11.6, 2.3 Hz, 2H), 2.27 (s, 3H), 2.07 (m, 1H), 1.36 (m, 2H), 1.25 (dtd, J=13.2, 11.6, 4.5 Hz, 2H); ¹³C NMR (126 MHz, DMSO) δ 152.8, 152.0, 150.5, 129.3, 118.4, 109.6, 66.9, 49.6, 35.7, 30.5, 9.8.

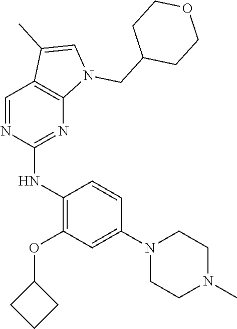 [SMILES: Cc1cn(CC2CCOCC2)c2nc(Nc3ccc(N4CCN(C)CC4)cc3OC3CCC3)ncc12]

N-(2-Cyclobutoxy-4-(4-methylpiperazin-1-yl)phenyl)-5-methyl-7-((tetrahydro-2H-pyran-4-yl)methyl)-7H-pyrrolo[2,3-d]pyrimidin-2-amine (SR8-005). SR8-005 was obtained as yellow solid (0.064 g, 77% yield) from SR7-183 (0.050 g, 0.188 mmol) and 2-cyclobutoxy-4-(4-methylpiperazin-1-yl)aniline (0.044 g, 0.169 mmol) by following the general procedure B. HPLC: >99% [t_{R}=15.2 min, gradient 5-95% MeOH and water (with 0.1% TFA), 20 min]. ¹H NMR (500 MHz, DMSO) δ 8.59 (s, 1H), 8.31 (d, J=8.8 Hz, 1H), 7.49 (s, 1H), 6.97 (s, 1H), 6.52 (dd, J=8.9, 2.6 Hz, 1H), 6.47 (d, J=2.6 Hz, 1H), 4.79 (m, 1H), 3.97 (d, J=7.1 Hz, 2H), 3.83 (ddd, J=11.5, 4.4, 2.0 Hz, 2H), 3.23 (td, J=11.7, 2.2 Hz, 2H), 3.09 (t, J=4.9 Hz, 4H), 2.45 (m, 6H), 2.26 (s, 3H), 2.23 (s, 3H), 2.16-2.04 (m, 3H), 1.85-1.75 (m, 1H), 1.72-1.59 (m, 1H), 1.42 (m, 2H), 1.33-1.20 (m, 2H); ¹³C NMR (126 MHz, DMSO) δ 156.2, 152.3, 149.6, 146.8, 146.6, 124.6, 122.9, 119.4, 112.9, 109.2, 107.7, 101.9, 71.6, 67.0, 55.1, 49.4, 49.2, 46.1, 40.6, 40.5, 40.4, 40.3, 40.4, 40.2, 40.1, 40.0, 39.8, 39.7, 39.5, 36.01 30.8, 13.2, 10.0. HRMS (ESI+): m/z calcd for C₂₈H₃₉N₆O₂ (M+H)⁺ 491.3129, found 491.3127 m/z calcd for C₂₈H₃₈N₆O₂Na (M+Na)⁺ 513.2948, found 513.2931; HPLC-MS (ESI+): m/z 491.3 [30% (M+H)⁺], 246.3 [100%, ((M+2H)²⁺/2)].

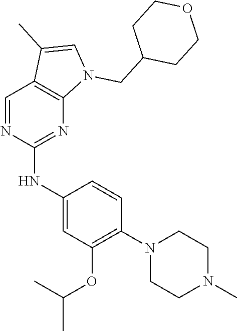 [SMILES: Cc1cn(CC2CCOCC2)c2nc(Nc3ccc(N4CCN(C)CC4)c(OC(C)C)c3)ncc12]

N-(3-isoPropoxy-4-(4-methylpiperazin-1-yl)phenyl)-5-methyl-7-((tetrahydro-2H-pyran-4-yl)methyl)-7H-pyrrolo[2,3-d]pyrimidin-2-amine (SR8-006). SR8-006 was obtained as a yellow foam (0.060 g, 65.7% yield) from SR7-183 (0.050 g, 0.188 mmol) and 3-isopropoxy-4-(4-methylpiperazin-1-yl)aniline (0.042 g, 0.169 mmol) by following the general procedure B. HPLC: >99% [t_{R}=15.1 min, gradient 5-95% MeOH and water (with 0.1% TFA), 20 min]. ¹H NMR (500 MHz, DMSO) δ 9.16 (s, 1H), 8.61 (s, 1H), 7.75 (d, J=2.4 Hz, 1H), 7.22 (dd, J=8.7, 2.4 Hz, 1H), 6.96 (s, 1H), 6.80 (d, J=8.6 Hz, 1H), 4.61 (sept, J=6.1 Hz, 1H), 3.96 (d, J=7.3 Hz, 2H), 3.82 (ddd, J=11.5, 4.6, 1.9 Hz, 2H), 3.23 (td, J=11.7, 2.1 Hz, 2H), 2.97 (s, 4H), 2.58 (s, 4H), 2.31 (s, 3H), 2.22 (s, 3H), 2.09 (m, 1H), 1.40 (ddd, J=12.6, 4.1, 1.8 Hz, 2H), 1.31 (d, J=6.0 Hz, 6H), 1.28-1.18 (m, 2H); ¹³C NMR (126 MHz, DMSO) δ 156.42, 152.14, 150.23, 149.6, 137.3, 124.2, 118.9, 112.7, 111.3, 109.2, 107.6, 70.1, 67.0, 55.3, 50.3, 49.0, 40.6, 40.5, 40.4, 40.3, 40.3, 40.2, 40.1, 40.0, 39.9, 39.8, 39.7, 39.5, 36.0, 30.7, 22.6, 21.5, 10.0. HRMS (ESI+): m/z calcd for C₂₇H₃₉N₆O₂ (M+H)⁺ 479.3129, found 479.3125 m/z calcd for C₂₇H₃₈N₆O₂Na (M+Na)⁺ 501.2948, found 501.2936; HPLC-MS (ESI+): m/z 479.2 [40% (M+H)⁺], 240.2 [100%, ((M+2H)²⁺/2)].

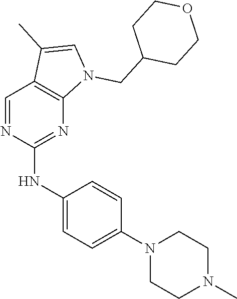 [SMILES: Cc1cn(CC2CCOCC2)c2nc(Nc3ccc(N4CCN(C)CC4)cc3)ncc12]

5-Methyl-N-(4-(4-methylpiperazin-1-yl)phenyl)-7-((tetrahydro-2H-pyran-4-yl)methyl)-7H-pyrrolo[2,3-d]pyrimidin-2-amine (SR8-008). SR8-008 was obtained as a white foam (0.058 g, 82% yield) from SR7-183 (0.050 g, 0.188 mmol) and 4(4-methylpiperazine-1-yl)aniline (0.032 g, 0.169 mmol) by following the general procedure B. HPLC: >99% [t_{R}=13.6 min, gradient 5-95% MeOH and water (with 0.1% TFA), 20 min]. ¹H NMR (500 MHz, DMSO) δ 9.08 (s, 1H), 8.59 (s, 1H), 7.71 (d, J=9.1 Hz, 2H), 6.93 (d, J=1.2 Hz, 1H), 6.88 (d, J=9.1 Hz, 2H), 3.95 (d, J=7.1 Hz, 2H), 3.83 (ddd, J=11.4, 4.4, 2.0 Hz, 2H), 3.24 (td, J=11.7, 2.1 Hz, 2H), 3.06 (t, J=5.0 Hz, 4H), 2.49 (bs, 4H), 2.25 (s, 3H), 2.22 (s, 3H), 2.15-2.04 (m, 1H), 1.42 (m, 2H), 1.34-1.20 (m, 2H); ¹³C NMR (126 MHz, DMSO) δ 156.6, 152.3, 149.4, 145.8, 134.5, 124.3, 119.5, 116.5, 112.7, 109.1, 67.0, 55.2, 49.5, 49.2, 46.2, 36.0, 30.8, 10.0. HRMS (ESI+): m/z calcd for C₂₄H₃₃N₆O (M+H)⁺ 421.2710, found 421.2707 m/z calcd for C₂₄H₃₂N₆ONa (M+Na)⁺ 443.2530, found 443.2524; HPLC-MS (ESI+): m/z 421.3 [40% (M+H)⁺], 211.2 [100%, ((M+2H)²⁺/2)].

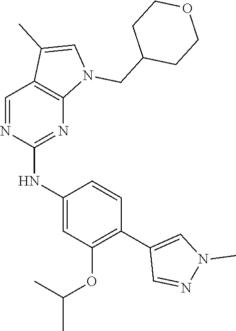 [SMILES: Cc1cn(CC2CCOCC2)c2nc(Nc3ccc(-c4cnn(C)c4)c(OC(C)C)c3)ncc12]

N-(3-isoPropoxy-4-(1-methyl-1H-pyrazol-4-yl)phenyl)-5-methyl-7-((tetrahydro-2H-pyran-4-yl)methyl)-7H-pyrrolo[2,3-d]pyrimidin-2-amine (SR8-009). SR8-009 was obtained as a white foam (0.040 g, 51% yield) from SR7-183 (0.050 g, 0.188 mmol) and 3-isopropoxy-4-(1-methyl-1H-pyrazol-4-yl)aniline (0.039 g, 0.169 mmol) by following the general procedure B. HPLC: >99% [t_{R}=17.4 min, gradient 5-95% MeOH and water (with 0.1% TFA), 20 min]. ¹H NMR (500 MHz, DMSO) δ 9.39 (s, 1H), 8.67 (s, 1H), 7.99-7.97 (m, 2H), 7.83 (d, J=0.8 Hz, 1H), 7.44 (d, J=8.4 Hz, 1H), 7.26 (dd, J=8.5, 2.1 Hz, 1H), 7.01 (d, J=1.2 Hz, 1H), 4.72 (sept, J=6.1 Hz, 1H), 4.01 (d, J=7.3 Hz, 2H), 3.87 (s, 3H), 3.84 (m, 2H), 3.24 (td, J=11.7, 2.1 Hz, 2H), 2.25 (d, J=1.2 Hz, 3H), 2.13 (tdd, J=15.4, 8.2, 2.8 Hz, 1H), 1.42 (s, 2H), 1.41 (d, J=6.0 Hz, 6H), 1.34-1.21 (m, 2H); ¹³C NMR (126 MHz, DMSO) δ 156.3, 153.9, 152.0, 149.6, 141.0, 137.4, 128.9, 127.5, 124.5, 118.9, 114.7, 112.9, 110.9, 109.3, 103.9, 70.0, 67.0, 49.1, 38.9, 36.0, 30.8, 22.48, 10.0. HRMS (ESI+): m/z calcd for C₂₆H₃₃N₆O₂ (M+H)⁺ 461.2660, found 461.2657 m/z calcd for C₂₆H₃₂N₆O₂Na (M+Na)⁺ 483.2479, found 483.2494; HPLC-MS (ESI+): m/z 461.3 [100% (M+H)⁺], 231.2 [10%, ((M+2H)²⁺/2)].

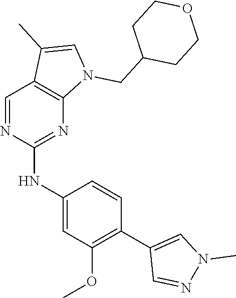 [SMILES: COc1cc(Nc2ncc3c(C)cn(CC4CCOCC4)c3n2)ccc1-c1cnn(C)c1]

N-(3-Methoxy-4-(1-methyl-1H-pyrazol-4-yl)phenyl)-5-methyl-7-((tetrahydro-2H-pyran-4-yl)methyl)-7H-pyrrolo[2,3-d]pyrimidin-2-amine (SR8-013). SR8-013 was obtained as yellow foam (0.049 g, 67% yield) from SR7-183 (0.050 g, 0.188 mmol) and 3-methoxy-4-(1-methyl-1H-pyrazol-4-yl)aniline (0.034 g, 0.169 mmol) by following the general procedure B. HPLC: >99% [t_{R}=16.6 min, gradient 5-95% MeOH and water (with 0.1% TFA), 20 min]. ¹H NMR (500 MHz, DMSO) δ 9.43 (s, 1H), 8.67 (s, 1H), 8.03 (d, J=2.0 Hz, 1H), 8.01 (d, J=0.8 Hz, 1H), 7.82 (d, J=0.8 Hz, 1H), 7.45 (d, J=8.4 Hz, 1H), 7.29 (dd, J=8.4, 2.1 Hz, 1H), 7.01 (d, J=1.3 Hz, 1H), 4.02 (d, J=7.3 Hz, 2H), 3.93 (s, 3H), 3.86 (s, 3H), 3.83 (ddd, J=11.6, 4.7, 1.9 Hz, 2H), 3.23 (td, J=11.7, 2.1 Hz, 2H), 2.25 (s, 3H), 2.17-2.07 (m, 1H), 1.45-1.35 (m, 2H), 1.33-1.21 (m, 2H); ¹³C NMR (126 MHz, DMSO) δ 156.2, 155.9, 151.9, 149.6, 141.2, 137.3, 129.1, 127.2, 124.7, 118.6, 113.7, 113.0, 110.8, 109.2, 101.9, 67.0, 55.6, 49.2, 38.9, 35.9, 30.8, 10.0. HRMS (ESI+): m/z calcd for C₂₄H₂₉N₆O₂ (M+H)⁺ 433.2347, found 433.2348 m/z calcd for C₂₄H₂₈N₆O₂Na (M+Na)⁺ 455.2166, found 455.2189; HPLC-MS (ESI+): m/z 433.2 [100% (M+H)⁺], 217.2 [10%, ((M+2H)²⁺/2)].

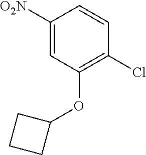 [SMILES: O=[N+]([O-])c1ccc(Cl)c(OC2CCC2)c1]

1-Chloro-2-cyclobutoxy-4-nitrobenzene (SR8-020). The cyclobutyl ether SR8-020 was obtained as a yellow solid (1.059 g 81% yield) from bromocyclobutane (0.856 g, 6.336 mmol) by following the general procedure 2. ¹H NMR (500 MHz, DMSO) δ 7.84 (dd, J=8.7, 2.5 Hz, 1H), 7.76 (d, J=8.7 Hz, 1H), 7.69 (d, J=2.5 Hz, 1H), 4.99 (m, 1H), 2.55-2.45 (m, 4H), 2.19-2.07 (m, 2H), 1.93-1.78 (m, 1H), 1.71 (m, 1H).

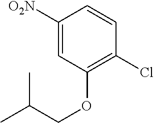

1-Chloro-2-isobutoxy-4-nitrobenzene (SR8-026). The isobutyl ether SR8-026 was obtained as a yellow solid (1.971 g 99% yield) from 1-bromo-2-methylpropane (1.421 g, 10.368 mmol) by following the general procedure 2. ¹H NMR (500 MHz, DMSO) δ 7.88 (d, J=2.5 Hz, 1H), 7.84 (dd, J=8.7, 2.6 Hz, 1H), 7.75 (d, J=8.6 Hz, 1H), 4.01 (d, J=6.4 Hz, 2H), 2.10 (dh, J=6.6, 6.5 Hz, 1H), 1.03 (d, J=6.7 Hz, 6H).

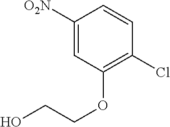 [SMILES: O=[N+]([O-])c1ccc(Cl)c(OCCO)c1]

2-(2-Chloro-5-nitrophenoxy)ethan-1-ol (SR8-030). The alcohol SR8-030 was obtained as a brown solid (1.050 g 84% yield) from 2-bromoethanol (1.574 g, 12.672 mmol) by following the general procedure 2. ¹H NMR (500 MHz, DMSO) δ 7.95 (d, J=2.5 Hz, 1H), 7.84 (dd, J=8.7, 2.6 Hz, 1H), 7.75 (d, J=8.7 Hz, 1H), 4.98 (t, J=5.3 Hz, 1H), 4.26 (dt, J=5.3, 4.4 Hz, 2H), 3.79 (t, J=4.6 Hz, 2H).

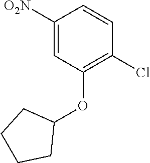 [SMILES: O=[N+]([O-])c1ccc(Cl)c(OC2CCCC2)c1]

1-Chloro-2-(cyclopentyloxy)-4-nitrobenzene (SR8-053). SR8-053 was obtained as a brown solid (1.621 g, 78% yield) from bromocyclopentane (1.931 g, 12.962 mmol) by following the general procedure 2. ¹H NMR (500 MHz, DMSO) δ 7.87 (d, J=2.5 Hz, 1H), 7.81 (dd, J=8.7, 2.5 Hz, 1H), 7.73 (d, J=8.7 Hz, 1H), 5.12 (tt, J=5.8, 3.0 Hz, 1H), 2.03-1.90 (m, 2H), 1.84-1.68 (m, 4H), 1.65-1.54 (m, 2H); ¹³C NMR (126 MHz, DMSO) δ 153.9, 147.6, 131.2, 130.0, 116.6, 109.7, 81.8, 32.5, 24.0.

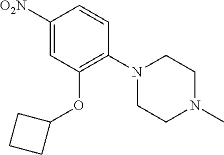 [SMILES: CN1CCN(c2ccc([N+](=O)[O-])cc2OC2CCC2)CC1]

1-(2-cyclobutoxy-4-nitrophenyl)-4-methylpiperazine (SR8-025). SR8-025 was obtained as a dark brown solid (1.165 g, 87% yield) by following the general procedure B. ¹H NMR (500 MHz, DMSO) δ 7.81 (dd, J=8.9, 2.6 Hz, 1H), 7.48 (d, J=2.6 Hz, 1H), 7.00 (d, J=8.9 Hz, 1H), 4.83 (p, J=7.1 Hz, 1H), 3.23 (t, J=5.0 Hz, 4H), 2.47 (m, 4H), 2.23 (s, 3H), 2.16-2.03 (m, 2H), 1.89-1.78 (m, 1H), 1.78-1.66 (m, 1H).

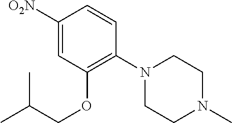 [SMILES: CC(C)COc1cc([N+](=O)[O-])ccc1N1CCN(C)CC1]

1-(2-isoButoxy-4-nitrophenyl)-4-methylpiperazine (SR8-028). SR8-028 was obtained as a brown solid (1.693 g, 89%) from SR8-026 (1.50 g, 6.531 mmol) and 1-methylpiperazine (0.851 g, 8.494 mmol) by following the general procedure B. ¹H NMR (500 MHz, DMSO) δ 7.83 (dd, J=8.9, 2.6 Hz, 1H), 7.66 (d, J=2.6 Hz, 1H), 7.00 (d, J=8.9 Hz, 1H), 3.89 (d, J=6.1 Hz, 2H), 3.22 (t, J=4.9 Hz, 4H), 2.47 (t, J=4.9 Hz, 4H), 2.23 (s, 3H), 2.09 (hept, J=6.6 Hz, 1H), 1.04 (d, J=6.7 Hz, 6H).

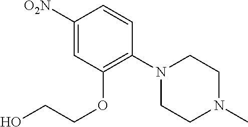 [SMILES: CN1CCN(c2ccc([N+](=O)[O-])cc2OCCO)CC1]

2-(2-(4-Methylpiperazin-1-yl)-5-nitrophenoxy)ethan-1-ol (SR8-034). The alcohol SR8-034 was obtained as a dark brown solid (0.514 g, 38% yield) by following the general procedure B. ¹H NMR (500 MHz, DMSO) δ 7.81 (dd, J=8.9, 2.6 Hz, 1H), 7.68 (d, J=2.6 Hz, 1H), 6.99 (d, J=8.9 Hz, 1H), 4.90 (t, J=5.2 Hz, 1H), 4.11 (m, 2H), 3.76 (t, J=5.0 Hz, 2H), 3.25 (d, J=5.1 Hz, 4H), 2.45 (d, J=5.2 Hz, 4H), 2.22 (s, 3H); ¹³C NMR (126 MHz, DMSO) δ 150.3, 148.1, 140.9, 118.3, 117.2, 108.2, 71.0, 60.0, 55.0, 49.6, 46.2.

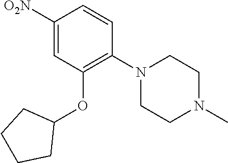 [SMILES: CN1CCN(c2ccc([N+](=O)[O-])cc2OC2CCCC2)CC1]

1-(2-(cyclopentyloxy)-4-nitrophenyl)-4-methylpiperazine (SR8-056). The ether SR8-056 was obtained as a dark brown solid (1.740 g, 86% yield) by following the general procedure B. ¹H NMR (500 MHz, DMSO) δ 7.81 (dd, J=8.9, 2.6 Hz, 1H), 7.64 (d, J=2.6 Hz, 1H), 6.99 (d, J=8.9 Hz, 1H), 5.00-4.94 (m, 1H), 3.19 (t, J=4.8 Hz, 4H), 2.46 (t, J=4.9 Hz, 4H), 2.22 (s, 3H), 1.97-1.87 (m, 2H), 1.83-1.59 (m, 6H).

### General Procedure 3: Reduction of Nitro Using Pd/C Under H₂

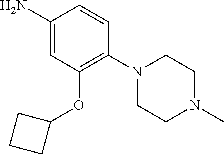 [SMILES: CN1CCN(c2ccc(N)cc2OC2CCC2)CC1]

3-Cyclobutoxy-4-(4-methylpiperazin-1-yl)aniline (SR8-029). The aniline SR8-029 was obtained as a black gum (0.904 g, 89% yield) following the general procedure 3. ¹H NMR (500 MHz, DMSO) δ 6.58 (d, J=8.2 Hz, 1H), 6.07 (d, J=2.4 Hz, 1H), 6.05 (dd, J=8.2, 2.4 Hz, 1H), 4.65 (bs, 2H), 4.52 (p, J=6.9 Hz, 1H), 2.81 (bs, 4H), 2.47-2.30 (m, 6H), 2.21 (s, 3H), 2.01 (m, 2H), 1.82-1.72 (m, 1H), 1.68-1.57 (m, 1H); ¹³C NMR (126 MHz, DMSO) δ 151.3, 145.1, 131.7, 119.7, 106.2, 101.6, 71.1, 55.5, 51.0, 46.2, 31.0, 13.3.

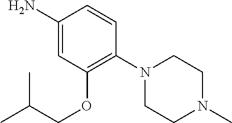 [SMILES: CC(C)COc1cc(N)ccc1N1CCN(C)CC1]

3-isoButoxy-4-(4-methylpiperazin-1-yl)aniline (SR8-039). The isobutyl ether SR8-039 was obtained as a black gum (1.261 g, 85% yield) following the general procedure 3. ¹H NMR (500 MHz, DMSO) δ 6.58 (d, J=8.2 Hz, 1H), 6.21 (d, J=2.4 Hz, 1H), 6.06 (dd, J=8.3, 2.4 Hz, 1H), 4.66 (bs, 2H), 3.63 (d, J=6.3 Hz, 2H), 2.82 (bs, 4H), 2.42 (bs, 4H), 2.20 (s, 3H), 2.01 (hept, J=6.6 Hz, 1H), 1.00 (d, J=6.8 Hz, 6H).

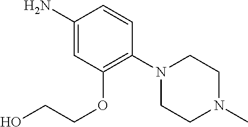 [SMILES: CN1CCN(c2ccc(N)cc2OCCO)CC1]

2-(5-Amino-2-(4-methylpiperazin-1-yl)phenoxy)ethan-1-ol (SR8-040). The aniline SR8-040 was obtained as a black gum (0.436 g, 98% yield) following the general procedure 3. ¹H NMR (500 MHz, DMSO) δ 6.61 (d, J=8.3 Hz, 1H), 6.24 (d, J=2.4 Hz, 1H), 6.10 (dd, J=8.4, 2.4 Hz, 1H), 4.85 (t, J=5.5 Hz, 1H), 4.69 (bs, 2H), 3.90 (t, J=5.2 Hz, 2H), 3.66 (q, J=5.3 Hz, 2H), 2.83 (bs, 4H), 2.41 (s, 4H), 2.20 (s, 3H).

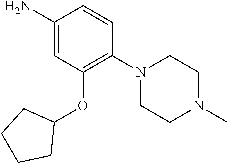 [SMILES: CN1CCN(c2ccc(N)cc2OC2CCCC2)CC1]

3-(Cyclopentyloxy)-4-(4-methylpiperazin-1-yl)aniline (SR8-059). The aniline SR8-059 was obtained as a black gum (1.352 g, 87% yield) following the general procedure 3. ¹H NMR (600 MHz, DMSO) δ 6.57 (d, J=8.3 Hz, 1H), 6.20 (d, J=2.4 Hz, 1H), 6.06 (dd, J=8.3, 2.4 Hz, 1H), 4.69 (d, J=5.6 Hz, 2H), 2.81 (bs, 4H), 2.46 (bs, 4H), 2.23 (s, 3H), 1.87-1.77 (m, 2H), 1.72 (m, 4H), 1.59 (m, 2H).

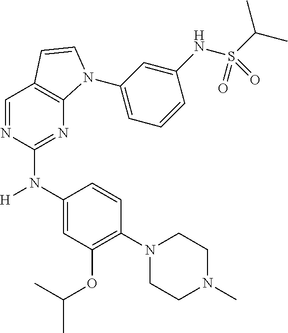 [SMILES: CC(C)Oc1cc(Nc2ncc3ccn(-c4cccc(NS(=O)(=O)C(C)C)c4)c3n2)ccc1N1CCN(C)CC1]

N-(3-(2-((3-isoPropoxy-4-(4-methylpiperazin-1-yl)phenyl)amino)-7H-pyrrolo[2,3-d]pyrimidin-7-yl)phenyl)propane-2-sulfonamide (SR8-080). The pyrrolopyrimidine SR8-080 was obtained as a yellow foam (0.157 g, 75% yield) from SR8-063 (0.130 g, 0.371 mmol) and 3-isopropoxy-4-(4-methylpiperazin-1-yl)aniline (0.090 g, 0.371 mmol) by following the general procedure A. HPLC: >99% [t_{R}=16.8 min, gradient 5-95% MeOH and water (with 0.1% TFA), 20 min]. ¹H NMR (500 MHz, DMSO) δ 10.05 (s, 1H), 9.28 (s, 1H), 8.77 (s, 1H), 7.58 (m, 1H), 7.53-7.49 (m, 3H), 7.48 (d, J=3.7 Hz, 1H), 7.30-7.23 (m, 2H), 6.79 (d, J=8.7 Hz, 1H), 6.66 (d, J=3.7 Hz, 1H), 4.33 (hept, J=6.1 Hz, 1H), 3.29 (m, 1H), 2.94 (bs, 4H), 2.53 (m, 4H), 2.33-2.19 (s, 3H), 1.23 (d, J=6.8 Hz, 6H), 1.15 (d, J=6.0 Hz, 6H); ¹³C NMR (126 MHz, DMSO) δ 156.8, 151.9, 151.6, 150.3, 140.2, 138.6, 136.8, 136.4, 130.6, 126.5, 119.2, 118.8, 117.2, 114.7, 112.8, 111.3, 107.1, 102.4, 69.9, 60.2, 55.4, 52.0, 50.4, 22.4, 16.6. HRMS (ESI+): m/z calcd for C₂₉H₃₈N₇O₃S (M+H)⁺ 564.2751, found 564.2748 m/z calcd for C₂₉H₃₇N₇O₃SNa (M+Na)⁺ 586.2571, found 586.2566; HPLC-MS (ESI): m/z 564.2 [100% (M+H)⁺], 562.2 [100%, (M−1)⁻].

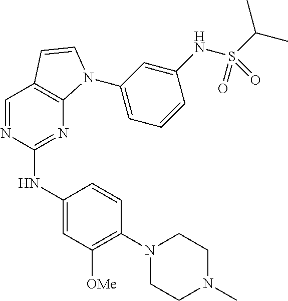 [SMILES: COc1cc(Nc2ncc3ccn(-c4cccc(NS(=O)(=O)C(C)C)c4)c3n2)ccc1N1CCN(C)CC1]

N-(3-(2-((3-Methoxy-4-(4-methylpiperazin-1-yl)phenyl)amino)-7H-pyrrolo[2,3-d]pyrimidin-7-yl)phenyl)propane-2-sulfonamide (SR8-082). The pyrrolopyrimidine SR8-082 was obtained as a yellow foam (0.056 g, 74% yield) from SR8-063 (0.050 g, 0.142 mmol) and 3-methoxy-4-(4-methylpiperazin-1-yl)aniline (0.031 g, 0.142 mmol) by following the general procedure A. HPLC: >99% [t_{R}=16.0 min, gradient 5-95% MeOH and water (with 0.1% TFA), 20 min]. ¹H NMR (500 MHz, DMSO) δ 10.04 (s, 1H), 9.31 (s, 1H), 8.79 (s, 1H), 7.59 (t, J=2.0 Hz, 1H), 7.50 (m, 4H), 7.29 (dd, J=8.6, 2.4 Hz, 1H), 7.25 (dt, J=7.1, 2.0 Hz, 1H), 6.81 (d, J=8.6 Hz, 1H), 6.67 (d, J=3.7 Hz, 1H), 3.59 (s, 3H), 3.35 (d, J=6.8 Hz, 1H), 2.91 (s, 4H), 2.51 (m, 4H), 2.27 (s, 3H), 1.24 (d, J=6.7 Hz, 6H); ¹³C NMR (126 MHz, DMSO) δ 156.8, 152.4, 151.9, 151.7, 140.2, 138.6, 136.9, 130.6, 126.5, 119.4, 118.5, 117.3, 114.8, 112.8, 110.8, 103.7, 102.4, 55.5, 55.3, 52.1, 50.6, 16.6.

HRMS (ESI+): m/z calcd for C₂₇H₃₄N₇O₃S (M+H)⁺ 536.2438, found 536.2447 m/z calcd for C₂₇H₃₃N₇O₃S Na (M+Na)⁺ 558.2258, found 558.2266; HPLC-MS (ESI): m/z 536.2 [90% (M+H)⁺], 534.2 [100%, (M−1)⁻].

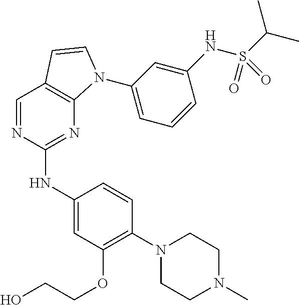 [SMILES: CC(C)S(=O)(=O)Nc1cccc(-n2ccc3cnc(Nc4ccc(N5CCN(C)CC5)c(OCCO)c4)nc32)c1]

N-(3-(2-((3-(2-Hydroxyethoxy)-4-(4-methylpiperazin-1-yl)phenyl)amino)-7H-pyrrolo[2,3-d]pyrimidin-7-yl)phenyl)propane-2-sulfonamide (SR8-083). The pyrrolopyrimidine SR8-083 was obtained as a pale yellow foam (0.042 g, 52% yield) from SR8-063 (0.050 g, 0.142 mmol) and 2-(5-amino-2-(4-methylpiperazin-1-yl)phenoxy)ethan-1-ol (SR8-040) (0.036 g, 0.142 mmol) by following the general procedure A. HPLC: >99% [t_{R}=15.5 min, gradient 5-95% MeOH and water (with 0.1% TFA), 20 min]. ¹H NMR (500 MHz, DMSO) δ 10.03 (s, 1H), 9.29 (s, 1H), 8.78 (s, 1H), 7.59 (t, J=2.1 Hz, 1H), 7.54 (m, 2H), 7.50 (d, J=3.8 Hz, 2H), 7.45 (d, J=2.4 Hz, 1H), 7.35 (dd, J=8.6, 2.4 Hz, 1H), 7.25 (m, 1H), 6.81 (d, J=8.6 Hz, 1H), 6.67 (d, J=3.7 Hz, 1H), 4.78 (d, J=6.2 Hz, 1H), 3.83 (t, J=5.0 Hz, 2H), 3.66 (t, J=4.7 Hz, 2H), 3.36 (m, 1H), 2.95 (s, 4H), 2.48-2.41 (m, 4H), 2.23 (s, 3H), 1.24 (d, J=6.7 Hz, 6H); ¹³C NMR (126 MHz, DMSO) δ 156.8, 151.9, 151.7, 140.2, 138.6, 136.7, 136.2, 130.6, 126.4, 119.2, 118.5, 117.3, 114.7, 112.9, 111.4, 105.9, 102.4, 70.6, 60.2, 55.5, 52.0, 50.7, 46.3, 16.6. HRMS (ESI+): m/z calcd for C₂₈H₃₆N₇O₄S (M+H)⁺ 566.2544, found 566.2529 m/z calcd for C₂₈H₃₅N₇O₄S Na (M+Na)⁺ 588.2363, found 588.2349; HPLC-MS (ESI): m/z 566.3 [100% (M+H)⁺], 564.2 [100%, (M−1)⁻].

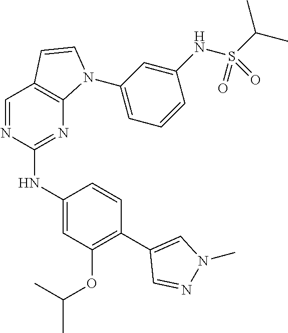 [SMILES: CC(C)Oc1cc(Nc2ncc3ccn(-c4cccc(NS(=O)(=O)C(C)C)c4)c3n2)ccc1-c1cnn(C)c1]

N-(3-(2-((3-isoPropoxy-4-(1-methyl-1H-pyrazol-4-yl)phenyl)amino)-7H-pyrrolo[2,3-d]pyrimidin-7-yl)phenyl)propane-2-sulfonamide (SR8-086). The pyrrolopyrimidine SR8-086 was obtained as a yellow foam (0.041 g, 53% yield) from SR8-063 (0.050 g, 0.142 mmol) and 3-isopropoxy-4-(1-methyl-1H-pyrazol-4-yl)aniline (0.033 g, 0.142 mmol) by following the general procedure A. HPLC: >96% [t_{R}=16.0 min, gradient 5-95% MeOH and water (with 0.1% TFA), 20 min]. ¹H NMR (500 MHz, DMSO) δ 10.09 (s, 1H), 9.51 (s, 1H), 8.83 (s, 1H), 7.95 (d, J=0.7 Hz, 1H), 7.81 (d, J=0.8 Hz, 1H), 7.75 (d, J=2.1 Hz, 1H), 7.59 (t, J=2.0 Hz, 1H), 7.57-7.47 (m, 3H), 7.44 (d, J=8.5 Hz, 1H), 7.28 (ddd, J=8.0, 2.2, 1.2 Hz, 1H), 7.25 (m, 1H), 6.69 (d, J=3.6 Hz, 1H), 4.31 (h, J=6.0 Hz, 1H), 3.85 (s, 3H), 3.29 (m, 1H), 1.20 (m, 12H); ¹³C NMR (126 MHz, DMSO) δ 156.6, 153.9, 151.9, 151.5, 140.5, 140.2, 138.6, 137.4, 130.7, 128.9, 127.4, 126.9, 119.3, 118.9, 117.4, 114.8, 114.8, 113.1, 110.9, 103.6, 102.3, 69.8, 52.0, 38.9, 22.3, 16.5. HRMS (ESI+): m/z calcd for C₂₈H₃₂N₇O₃S (M+H)⁺ 546.2282, found 546.2299 m/z calcd for C₂₈H₃₁N₇O₃S Na (M+Na)⁺ 568.2101, found 568.2123; HPLC-MS (ESI): m/z 546.0 [100% (M+H)⁺], 544.2 [100%, (M−1)⁻].

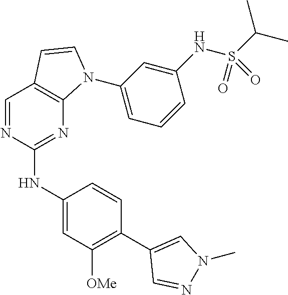 [SMILES: COc1cc(Nc2ncc3ccn(-c4cccc(NS(=O)(=O)C(C)C)c4)c3n2)ccc1-c1cnn(C)c1]

N-(3-(2-((3-Methoxy-4-(1-methyl-1H-pyrazol-4-yl)phenyl)amino)-7H-pyrrolo[2,3-d]pyrimidin-7-yl)phenyl)propane-2-sulfonamide (SR8-089). The pyrrolopyrimidine SR8-089 was obtained as a yellow foam (0.053 g, 72% yield) from SR8-063 (0.050 g, 0.142 mmol) and 3-methoxy-4-(1-methyl-1H-pyrazol-4-yl)aniline (0.029 g, 0.142 mmol) by following the general procedure A. HPLC: >96% [t_{R}=15.3 min, gradient 5-95% MeOH and water (with 0.1% TFA), 20 min]. ¹H NMR (500 MHz, DMSO) δ 10.07 (s, 1H), 9.54 (s, 1H), 8.84 (s, 1H), 7.98 (s, 1H), 7.80 (s, 1H), 7.70 (d, J=2.0 Hz, 1H), 7.59 (t, J=1.6 Hz, 1H), 7.52 (m, 3H), 7.46 (d, J=8.4 Hz, 1H), 7.34 (dd, J=8.5, 2.1 Hz, 1H), 7.27 (tq, J=5.8, 2.1 Hz, 1H), 6.70 (d, J=3.6 Hz, 1H), 3.85 (s, 3H), 3.64 (s, 3H), 3.30 (d, J=6.7 Hz, 1H), 1.22 (d, J=6.8 Hz, 6H); ¹³C NMR (126 MHz, DMSO) δ 156.6, 155.8, 151.9, 151.6, 140.6, 140.2, 138.6, 137.3, 130.7, 129.2, 127.2, 126.9, 119.5, 118.5, 117.4, 114.9, 114.1, 113.1, 111.0, 102.3, 102.0, 55.4, 52.1, 38.9, 16.5. HRMS (ESI+): m/z calcd for C₂₆H₂₈N₇O₃S (M+H)⁺ 518.1969, found 518.1979 m/z calcd for C₂₆H₂₇N₇O₃SNa (M+Na)⁺ 540.1788, found 540.1779; HPLC-MS (ESI): m/z 518.0 [100% (M+H)⁺], 516.2 [100%, (M−1)⁻].

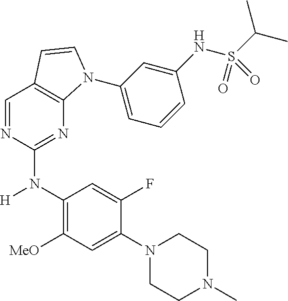 [SMILES: COc1cc(N2CCN(C)CC2)c(F)cc1Nc1ncc2ccn(-c3cccc(NS(=O)(=O)C(C)C)c3)c2n1]

N-(3-(2-((5-Fluoro-2-methoxy-4-(4-methylpiperazin-1-yl)phenyl)amino)-7H-pyrrolo[2,3-d]pyrimidin-7-yl)phenyl)propane-2-sulfonamide (SR8-093). The pyrrolopyrimidine SR8-093 was obtained as a pale yellow foam (0.059 g, 75% yield) from SR8-063 (0.050 g, 0.142 mmol) and 5-fluoro-2-methoxy-4-(4-methylpiperazin-1-yl)aniline (0.034 g, 0.142 mmol) by following the general procedure A. HPLC: >99% [t_{R}=17.7 min, gradient 5-95% MeOH and water (with 0.1% TFA), 20 min]. ¹H NMR (500 MHz, DMSO) δ 10.03 (s, 1H), 8.83 (s, 1H), 8.28 (d, J=15.0 Hz, 1H), 7.77 (d, J=1.2 Hz, 1H), 7.70 (t, J=2.1 Hz, 1H), 7.61 (d, J=3.7 Hz, 1H), 7.58 (ddd, J=8.0, 2.1, 1.0 Hz, 1H), 7.50 (t, J=8.1 Hz, 1H), 7.25 (ddd, J=8.0, 2.1, 1.0 Hz, 1H), 6.75-6.67 (m, 2H), 3.88 (s, 3H), 3.37 (m, 1H), 3.02 (bs, 4H), 2.59-2.50 (bs, 4H), 2.26 (bs, 3H), 1.28 (d, J=6.9 Hz, 6H); ¹⁹F NMR (471 MHz, DMSO) δ −130.84 (dd, J=15.1, 8.1 Hz); HRMS (ESI+): m/z calcd for C₂₇H₃₃FN₇O₃S (M+H)⁺ 554.2344, found 554.2361 m/z calcd for C₂₇H₃₂FN₇O₃SNa (M+Na)⁺ 576. 2164, found 576.2176; HPLC-MS (ESI): m/z 554.0 [100% (M+H)⁺], 552.2 [100%, (M−1)⁻].

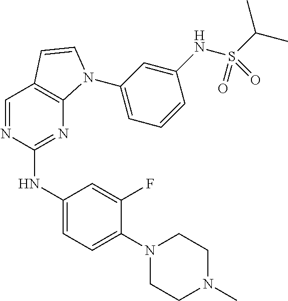 [SMILES: CC(C)S(=O)(=O)Nc1cccc(-n2ccc3cnc(Nc4ccc(N5CCN(C)CC5)c(F)c4)nc32)c1]

N-(3-(2-((3-Fluoro-4-(4-methylpiperazin-1-yl)phenyl)amino)-7H-pyrrolo[2,3-d]pyrimidin-7-yl)phenyl)propane-2-sulfonamide (SR8-097). The pyrrolopyrimidine SR8-097 was obtained as a pale yellow foam (0.056 g, 75% yield) from SR8-063 (0.050 g, 0.142 mmol) and 3-fluoro-4-(4-methylpiperazin-1-yl)aniline (0.030 g, 0.142 mmol) by following the general procedure A. HPLC: >99% [t_{R}=16.4 min, gradient 5-95% MeOH and water (with 0.1% TFA), 20 min]. ¹H NMR (500 MHz, DMSO) δ 10.04 (s, 1H), 9.54 (s, 1H), 8.82 (s, 1H), 7.77 (dd, J=15.6, 2.5 Hz, 1H), 7.63 (t, J=2.1 Hz, 1H), 7.58 (ddd, J=7.9, 2.0, 1.0 Hz, 1H), 7.55 (d, J=3.7 Hz, 1H), 7.52 (t, J=8.0 Hz, 1H), 7.50-7.46 (m, 1H), 7.26 (ddd, J=8.0, 2.1, 1.1 Hz, 1H), 6.97 (dd, J=10.1, 8.8 Hz, 1H), 6.69 (d, J=3.7 Hz, 1H), 3.37 (m, 1H), 2.95 (bs, 4H), 2.48 (bs, 4H), 2.26 (s, 3H), 1.26 (d, J=6.7 Hz, 6H); ¹⁹F NMR (471 MHz, DMSO) δ −122.13 (dd, J=15.7, 10.0 Hz); HRMS (ESI+): m/z calcd for C₂₆H₃₁FN₇O₂S (M+H)⁺ 524.2238, found 524.2242 m/z calcd for C₂₆H₃₀FN₇O₂SNa (M+Na)⁺ 546.2058, found 546.2054; HPLC-MS (ESI): m/z 524.0 [100% (M+H)⁺], 522.2 [100%, (M−1)⁻].

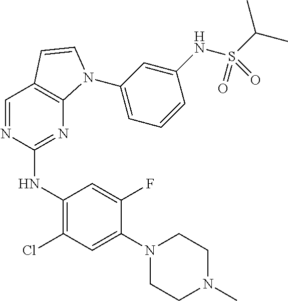 [SMILES: CC(C)S(=O)(=O)Nc1cccc(-n2ccc3cnc(Nc4cc(F)c(N5CCN(C)CC5)cc4Cl)nc32)c1]

N-(3-(2-((2-Chloro-5-fluoro-4-(4-methylpiperazin-1-yl)phenyl)amino)-7H-pyrrolo[2,3-d]pyrimidin-7-yl)phenyl)propane-2-sulfonamide (SR8-114). The pyrrolopyrimidine SR8-114 was obtained as a pale yellow foam (0.036 g, 63% yield) from SR8-063 (0.040 g, 0.114 mmol) and SR8-120 (0.025 g, 0.102 mmol) by following the general procedure A. HPLC: >99% [t_{R}=15.1 min, gradient 5-95% MeOH and water (with 0.1% TFA), 20 min]. ¹H NMR (500 MHz, DMSO) δ 9.99 (s, 1H), 8.82 (s, 1H), 8.23 (s, 1H), 8.08 (d, J=14.9 Hz, 1H), 7.62 (t, J=2.1 Hz, 1H), 7.60 (d, J=3.7 Hz, 1H), 7.57 (ddd, J=8.0, 2.1, 0.9 Hz, 1H), 7.46 (t, J=8.1 Hz, 1H), 7.23 (ddd, J=8.1, 2.1, 0.9 Hz, 1H), 7.10 (d, J=8.8 Hz, 1H), 6.72 (d, J=3.7 Hz, 1H), 3.35 (sept, J=6.8 Hz, 1H), 3.00 (t, J=4.8 Hz, 4H), 2.49 (bs, 4H), 2.25 (s, 3H), 1.25 (d, J=6.8 Hz, 6H); ¹⁹F NMR (471 MHz, DMSO) δ −122.40 (dd, J=15.0, 8.9 Hz); HRMS (ESI+): m/z calcd for C₂₆H₃₀ClFN₇O₂S (M+H)⁺ 558.1849, found 558.1839 m/z calcd for C₂₆H₂₉ClFN₇O₂SNa (M+Na)⁺ 580.1668, found 580.1645; HPLC-MS (ESI): m/z 557.9 [100% (M+H)⁺], 557.2 [100%, (M−1)⁻].

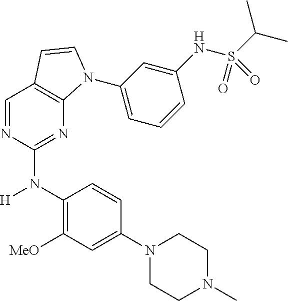 [SMILES: COc1cc(N2CCN(C)CC2)ccc1Nc1ncc2ccn(-c3cccc(NS(=O)(=O)C(C)C)c3)c2n1]

N-(3-(2-((2-Methoxy-4-(4-methylpiperazin-1-yl)phenyl)amino)-7H-pyrrolo[2,3-d]pyrimidin-7-yl)phenyl)propane-2-sulfonamide (SR8-136). The pyrrolopyrimidine SR8-136 was obtained as a yellow foam (0.050 g, 74% yield) from AM1-063 (0.050 g, 0.142 mmol) and 2-methoxy-4-(4-methylpiperidine-1-yl)aniline (0.028 g, 0.128 mmol) by following the general procedure A. HPLC: >99% [t_{R}=8.9 min, isocratic 40% MeOH and 60% water (with 0.1% TFA), 20 min]. ¹H NMR (500 MHz, DMSO) δ 10.01 (s, 1H), 8.74 (s, 1H), 8.09 (d, J=8.8 Hz, 1H), 7.77 (t, J=2.1 Hz, 1H), 7.67 (s, 1H), 7.55 (d, J=3.7 Hz, 1H), 7.53-7.47 (m, 2H), 7.21 (ddd, J=7.6, 2.2, 1.4 Hz, 1H), 6.66 (d, J=3.7 Hz, 1H), 6.64 (d, J=2.6 Hz, 1H), 6.51 (dd, J=8.8, 2.6 Hz, 1H), 3.84 (s, 3H), 3.36 (m, 1H), 3.11 (m, 4H), 2.48 (m, 4H), 2.24 (s, 3H), 1.27 (d, J=6.8 Hz, 6H); ¹³C NMR (126 MHz, DMSO) δ 156.9, 151.8, 149.9, 147.4, 140.0, 138.6, 130.6, 126.1, 121.9, 120.7, 118.5, 117.1, 114.3, 113.2, 107.5, 102.6, 100.6, 56.2, 55.2, 52.1, 49.4, 46.2, 16.6. HRMS (ESI+): m/z calcd for C₂₇H₃₄N₇O₃S (M+H)⁺ 536.2438, found 536.2423 m/z calcd for C₂₇H₃₃N₇O₃S Na (M+Na)⁺ 558.2258, found 558.2245; HPLC-MS (ESI+): m/z 536.1 [70% (M+H)⁺], 268.6 [100%, ((M+2H)²⁺/2)].

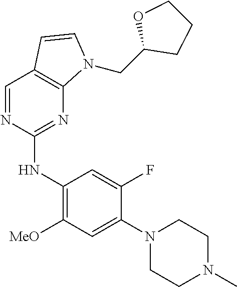 [SMILES: COc1cc(N2CCN(C)CC2)c(F)cc1Nc1ncc2ccn(C[C@H]3CCCO3)c2n1]

(R)—N-(5-fluoro-2-methoxy-4-(4-methylpiperazin-1-yl)phenyl)-7-((tetrahydrofuran-2-yl)methyl)-7H-pyrrolo[2,3-d]pyrimidin-2-amine (SR8-090). The pyrrolopyrimidine SR8-090 was obtained as a yellow foam (0.043 g, 52% yield) from SR8-088 (0.050 g, 0.210 mmol) and 5-fluoro-2-methoxy-4-(4-methylpiperazin-1-yl)aniline (0.045 g, 0.189 mmol) by following the general procedure B. HPLC: >99% [t_{R}=14.7 min, gradient 5-95% MeOH and water (with 0.1% TFA), 20 min]. ¹H NMR (500 MHz, DMSO) δ 8.69 (s, 1H), 8.36 (d, J=15.2 Hz, 1H), 7.68 (d, J=1.2 Hz, 1H), 7.29 (d, J=3.6 Hz, 1H), 6.71 (d, J=8.2 Hz, 1H), 6.45 (d, J=3.5 Hz, 1H), 4.25 (m, 1H), 4.18 (d, J=5.7 Hz, 2H), 3.90 (s, 3H), 3.79 (dt, J=8.2, 6.6 Hz, 1H), 3.64 (dt, J=8.2, 6.9 Hz, 1H), 3.02 (bs, 4H), 2.50 (bs, 4H), 2.26 (s, 3H), 1.99-1.88 (m, 1H), 1.84-1.76 (m, 2H), 1.69-1.57 (m, 1H); ¹⁹F NMR (471 MHz, DMSO) δ −131.16 (dd, J=15.2, 8.2 Hz); HRMS (ESI+): m/z calcd for C₂₃H₃₀FN₆O₂ (M+H)⁺ 441.2409, found 441.2411 m/z calcd for C₂₃H₂₉FN₆O₂Na (M+Na)⁺ 463.2228, found 463.2245; HPLC-MS (ESI+): m/z 441.0 [40% (M+H)⁺], 221.1 [100%, ((M+2H)²⁺/2)].

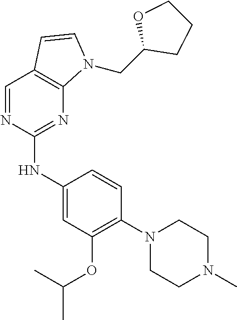 [SMILES: CC(C)Oc1cc(Nc2ncc3ccn(C[C@H]4CCCO4)c3n2)ccc1N1CCN(C)CC1]

(R)—N-(3-isoPropoxy-4-(4-methylpiperazin-1-yl)phenyl)-7-((tetrahydrofuran-2-yl)methyl)-7H-pyrrolo[2,3-d]pyrimidin-2-amine (SR8-091). The pyrrolopyrimidine SR8-091 was obtained as a pale yellow foam (0.059 g, 69% yield) from SR8-088 (0.050 g, 0.210 mmol) and 3-isopropoxy-4-(4-methylpiperazin-1-yl)aniline (0.047 g, 0.189 mmol) by following the general procedure B. HPLC: >98% [t_{R}=14.8 min, gradient 5-95% MeOH and water (with 0.1% TFA), 20 min]. ¹H NMR (500 MHz, DMSO) δ 9.20 (s, 1H), 8.65 (s, 1H), 7.74 (d, J=2.4 Hz, 1H), 7.28-7.18 (m, 2H), 6.81 (d, J=8.7 Hz, 1H), 6.41 (d, J=3.5 Hz, 1H), 4.60 (hept, J=6.1 Hz, 1H), 4.29-4.13 (m, 3H), 3.79 (ddd, J=8.2, 7.1, 6.0 Hz, 1H), 3.64 (ddd, J=8.2, 7.3, 6.4 Hz, 1H), 2.96 (bs, 4H), 2.66-2.51 (m, 4H), 2.29 (bs, 3H), 1.97-1.84 (m, 1H), 1.84-1.70 (m, 2H), 1.65-1.53 (m, 1H), 1.32 (dd, J=6.0 Hz, 6H). HRMS (ESI+): m/z calcd for C₂₅H₃₅N₆O₂ (M+H)⁺ 451.2816, found 451.2828 m/z calcd for C₂₅H₃₄N₆O₂Na (M+Na)⁺ 473.2635, found 473.2637; HPLC-MS (ESI+): m/z 451.1 [30% (M+H)⁺], 226.1 [100%, ((M+2H)²⁺/2)].

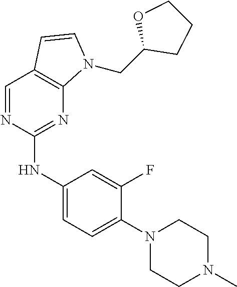 [SMILES: CN1CCN(c2ccc(Nc3ncc4ccn(C[C@H]5CCCO5)c4n3)cc2F)CC1]

(R)—N-(3-Fluoro-4-(4-methylpiperazin-1-yl)phenyl)-7-((tetrahydrofuran-2-yl)methyl)-7H-pyrrolo[2,3-d]pyrimidin-2-amine (SR8-095). The pyrrolopyrimidine SR8-095 was obtained as a pale yellow foam (0.036 g, 46% yield) from SR8-088 (0.050 g, 0.210 mmol) and 3-fluoro-4-(4-methylpiperazin-1-yl)aniline (0.040 g, 0.189 mmol) by following the general procedure B. HPLC: >98% [t_{R}=13.7 min, gradient 5-95% MeOH and water (with 0.1% TFA), 20 min]. ¹H NMR (500 MHz, DMSO) δ 9.43 (s, 1H), 8.68 (s, 1H), 7.88 (dd, J=15.8, 2.5 Hz, 1H), 7.50-7.41 (m, 1H), 7.26 (d, J=3.6 Hz, 1H), 6.97 (dd, J=10.1, 8.8 Hz, 1H), 6.43 (d, J=3.6 Hz, 1H), 4.25 (hept, J=6.0 Hz, 1H), 4.18 (d, J=5.6 Hz, 2H), 3.79 (dt, J=8.3, 6.6 Hz, 1H), 3.64 (dt, J=8.2, 6.9 Hz, 1H), 2.96 (bs, J=4.9 Hz, 4H), 2.45-2.50 (bs, 4H), 2.25 (s, 3H), 1.99-1.87 (m, 1H), 1.85-1.75 (m, 2H), 1.68-1.57 (m, 1H); ¹⁹F NMR (471 MHz, DMSO) δ −122.33 (dd, J=15.8, 10.1 Hz); HRMS (ESI+): m/z calcd for C₂₂H₂₈FN₆O (M+H)⁺ 411. 2303, found 411.2309 m/z calcd for C₂₂H₂₇FN₆ONa (M+Na)⁺ 433.2123, found 433.2211; HPLC-MS (ESI+): m/z 411.0 [40% (M+H)⁺], 206.1 [100%, ((M+2H)²⁺/2)].

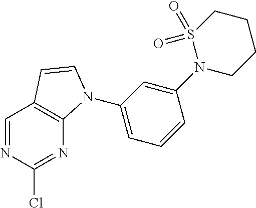 [SMILES: O=S1(=O)CCCCN1c1cccc(-n2ccc3cnc(Cl)nc32)c1]

2-(3-(2-Chloro-7H-pyrrolo[2,3-d]pyrimidin-7-yl)phenyl)-1,2-thiazinane 1,1-dioxide (SR8-127). 2-Chloro-7H-(2,3-d)pyrrolopyrimidine (0.156 g, 1.02 mmol), SR8-117 (0.378 g, 1.122 mmol), K₃PO₄ (0.650 g, 3.06 mmol) were added to a pressure flask along with dry 1,4-dioxane (8 mL). To the mixture, 1,2-diaminocyclohexane (0.047 g, 0.408 mmol) and CuI (0.039 g, 0.203 mmol) were added. The mixture was stirred at 100° C. for 24 h, cooled and filtered through Celite. The filtrate was concentrated under reduced pressure. The resulting residue was diluted with EtOAc and washed with water (25 mL) and brine (25 mL). The organic layer was dried (Na₂SO₄) and concentrated under vacuum. Purification by column chromatography (0-15% gradient elution, MeOH:DCM) afforded SR8-127 as an off-white solid (0.254 g, 70% yield). ¹H NMR (500 MHz, DMSO) δ 9.07 (s, 1H), 8.08 (d, J=3.8 Hz, 1H), 7.78-7.70 (m, 2H), 7.61 (t, J=8.4 Hz, 1H), 7.41 (ddd, J=8.2, 2.0, 1.0 Hz, 1H), 6.94 (d, J=3.7 Hz, 1H), 3.81-3.73 (m, 2H), 3.37-3.33 (m, 2H), 2.22-2.14 (m, 2H), 1.91-1.81 (m, 2H)¹³C NMR (126 MHz, DMSO) δ 153.4, 152.7, 151.6, 142.2, 137.2, 131.4, 130.2, 125.8, 122.5, 122.1, 119.2, 102.4, 53.6, 50.4, 24.4, 23.7. HPLC-MS (ESI+): m/z 362.9 [100% (M+H)⁺], 746.8 [60%, (2M+Na)⁺].

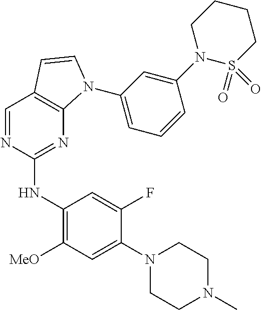 [SMILES: COc1cc(N2CCN(C)CC2)c(F)cc1Nc1ncc2ccn(-c3cccc(N4CCCCS4(=O)=O)c3)c2n1]

2-(3-(2-((5-Fluoro-2-methoxy-4-(4-methylpiperazin-1-yl)phenyl)amino)-7H-pyrrolo[2,3-d]pyrimidin-7-yl)phenyl)-1,2-thiazinane 1,1-dioxide (SR8-131). The pyrrolopyrimidine SR8-131 was obtained as a brown foam (0.034 g, 46% yield) from SR8-127 (0.050 g, 0.138 mmol) and 5-fluoro-2-methoxy-4-(4-methylpiperazin-1-yl)aniline (0.033 g, 0.138 mmol) by following the general procedure A. HPLC: >99% [t_{R}=17.2 min, gradient 5-95% MeOH and water (with 0.1% TFA), 20 min]. ¹H NMR (500 MHz, DMSO) δ 8.82 (s, 1H), 8.18 (d, J=14.9 Hz, 1H), 7.89 (ddd, J=8.1, 2.1, 0.9 Hz, 1H), 7.86 (s, 1H), 7.73-7.68 (m, 2H), 7.57 (t, J=8.1 Hz, 1H), 7.36 (ddd, J=8.1, 2.1, 0.9 Hz, 1H), 6.72 (d, J=3.8 Hz, 1H), 6.70 (d, J=8.2 Hz, 1H), 3.88 (s, 3H), 3.78-3.73 (m, 2H), 3.28 (m, 2H), 3.01 (m, 4H), 2.48 (m, 4H), 2.24 (s, 3H), 2.21-2.12 (m, 2H), 1.87-1.77 (m, 2H); ¹⁹F NMR (471 MHz, DMSO) δ −131.18 (dd, J=15.0, 8.1 Hz); HRMS (ESI+): m/z calcd for C₂₈H₃₃FN₇O₃S (M+H)⁺ 566.2344, found 566.2329 m/z calcd for C₂₈H₃₂FN₇O₃S Na (M+Na)⁺ 588.2164, found 588.2143; HPLC-MS (ESI+): m/z 566.0 [90% (M+H)⁺], 283.6 [100%, ((M+2H)²⁺/2)].

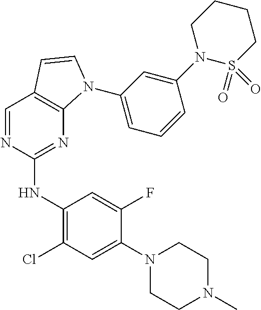 [SMILES: CN1CCN(c2cc(Cl)c(Nc3ncc4ccn(-c5cccc(N6CCCCS6(=O)=O)c5)c4n3)cc2F)CC1]

2-(3-(2-((2-Chloro-5-fluoro-4-(4-methylpiperazin-1-yl)phenyl)amino)-7H-pyrrolo[2,3-d]pyrimidin-7-yl)phenyl)-1,2-thiazinane 1,1-dioxide (SR8-134). The pyrrolopyrimidine SR8-134 was obtained as a beige foam (0.030 g, 42% yield) from SR8-127 (0.050 g, 0.138 mmol) and SR8-120 (0.030 g, 0.124 mmol) by following the general procedure A. HPLC: >98% [t_{R}=17.5 min, gradient 5-95% MeOH and water (with 0.1% TFA), 20 min]. ¹H NMR (500 MHz, DMSO) δ 8.82 (s, 1H), 8.41 (s, 1H), 7.94 (d, J=14.7 Hz, 1H), 7.84 (ddd, J=8.1, 2.3, 0.9 Hz, 1H), 7.73-7.70 (m, 2H), 7.52 (t, J=8.1 Hz, 1H), 7.33 (ddd, J=8.1, 2.2, 1.0 Hz, 1H), 7.10 (d, J=8.8 Hz, 1H), 6.72 (d, J=3.7 Hz, 1H), 3.76-3.67 (m, 2H), 3.31 (m, 2H), 3.00 (m, 4H), 2.47 (m, 4H), 2.23 (s, 3H), 2.22-2.12 (m, 2H), 1.88-1.77 (m, 2H); ¹⁹F NMR (471 MHz, DMSO) δ −122.76 (dd, J=14.7, 8.8 Hz); HRMS (ESI+): m/z calcd for C₂₇H₃₀ClFN₇O₂S (M+H)⁺ 570.1849, found 570.1832 m/z calcd for C₂₇H₂₉ClFN₇O₂S Na (M+Na)⁺ 592.1668, found 592.1653; HPLC-MS (ESI+): m/z 570.0 [100% (M+H)⁺], 285.6 [80%, ((M+2H)²⁺/2)].

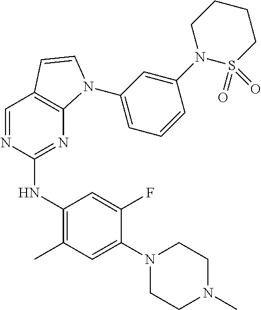 [SMILES: Cc1cc(N2CCN(C)CC2)c(F)cc1Nc1ncc2ccn(-c3cccc(N4CCCCS4(=O)=O)c3)c2n1]

2-(3-(2-((5-Fluoro-2-methyl-4-(4-methylpiperazin-1-yl)phenyl)amino)-7H-pyrrolo[2,3-d]pyrimidin-7-yl)phenyl)-1,2-thiazinane 1,1-dioxide (SR8-155). The pyrrolopyrimidine SR8-155 was obtained as a beige foam (0.030 g, 42% yield) from SR8-127 (0.050 g, 0.138 mmol) and 5-fluoro-2-methyl-4-(4-methylpiperazin-1-yl)aniline (0.028 g, 0.124 mmol) by following the general procedure A. HPLC: >99% [t_{R}=15.8 min, gradient 5-95% MeOH and water (with 0.1% TFA), 20 min]. ¹H NMR (500 MHz, DMSO) δ 8.74 (s, 1H), 8.47 (s, 1H), 7.81 (ddd, J=8.2, 2.2, 0.9 Hz, 1H), 7.71 (t, J=2.1 Hz, 1H), 7.64 (d, J=3.7 Hz, 1H), 7.52 (d, J=14.8 Hz, 1H), 7.48 (t, J=8.1 Hz, 1H), 7.29 (ddd, J=8.1, 2.1, 0.9 Hz, 1H), 6.85 (d, J=9.9 Hz, 1H), 6.66 (d, J=3.7 Hz, 1H), 3.66 (m, 2H), 3.29 (m, 2H), 2.98 (m, 4H), 2.48 (m, 4H), 2.23 (s, 3H), 2.20 (s, 3H), 2.19-2.11 (m, 2H), 1.83-1.75 (m, 2H); ¹⁹F NMR (471 MHz, DMSO) δ −126.28 (dd, J=14.8, 9.7 Hz); HRMS (ESI+): m/z calcd for C₂₈H₃₃FN₇O₂S (M+H)⁺ 550.2395, found 550.2391 m/z calcd for C₂₈H₃₂FN₇O₂S Na (M+Na)⁺ 572.2214, found 572.2204; HPLC-MS (ESI+): m/z 550.0 [90% (M+H)⁺], 275.6 [100%, ((M+2H)²⁺/2)].

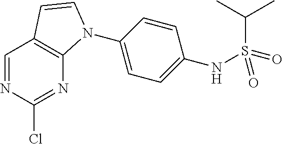 [SMILES: CC(C)S(=O)(=O)Nc1ccc(-n2ccc3cnc(Cl)nc32)cc1]

N-(4-(2-Chloro-7H-pyrrolo[2,3-d]pyrimidin-7-yl)phenyl)propane-2-sulfonamide (SR8-138). The sulfonamide SR8-138 was obtained as a foam (0.328 g, 68%) from (2-chloro-7H-(2,3-d)pyrrolopyrimidine (0.212 g, 1.383 mmol) and N-(4-iodophenyl)propane-2-sulfonamide (0.500 g, 1.537 mmol) by following the method described for SR8-127. ¹H NMR (500 MHz, DMSO) δ 10.03 (s, 1H), 9.05 (s, 1H), 7.98 (d, J=3.7 Hz, 1H), 7.72 (d, J=8.9 Hz, 2H), 7.43 (d, J=8.8 Hz, 2H), 6.91 (d, J=3.7 Hz, 1H), 3.32 (m, 1H), 1.29 (d, J=6.8 Hz, 6H); ¹³C NMR (126 MHz, DMSO) δ 153.3, 152.6, 151.5, 138.3, 132.2, 131.6, 125.6, 120.3, 118.9, 101.9, 52.0, 16.6. HPLC-MS (ESI+): m/z 350.9 [100% (M+H)⁺], 372.9 [60%, (M+Na)⁺].

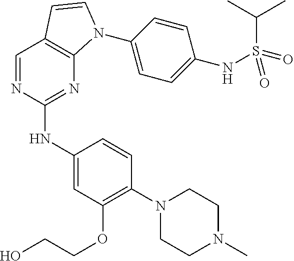 [SMILES: CC(C)S(=O)(=O)Nc1ccc(-n2ccc3cnc(Nc4ccc(N5CCN(C)CC5)c(OCCO)c4)nc32)cc1]

N-(4-(2-((3-(2-Hydroxyethoxy)-4-(4-methylpiperazin-1-yl)phenyl)amino)-7H-pyrrolo[2,3-d]pyrimidin-7-yl)phenyl)propane-2-sulfonamide (SR8-142). The pyrrolopyrimidine SR8-142 was obtained as an off-white foam (0.041 g, 57% yield) from SR8-138 (0.050 g, 0.142 mmol) and SR8-040 (0.032 g, 0.128 mmol) by following the general procedure A. HPLC: >99% [t_{R}=12.5 min, gradient 20-95% MeOH and water (with 0.1% TFA), 20 min]. 1H NMR (500 MHz, DMSO) δ 9.95 (s, 1H), 9.28 (s, 1H) 8.77 (s, 1H), 7.81 (m, 2H), 7.55 (d, J=3.7 Hz, 1H), 7.48 (d, J=2.4 Hz, 1H), 7.41 (m, 2H), 7.31 (dd, J=8.6, 2.4 Hz, 1H), 6.78 (d, J=8.6 Hz, 1H), 6.64 (d, J=3.7 Hz, 1H), 4.80 (bs, 1H), 3.90 (t, J=5.0 Hz, 2H), 3.69 (t, J=4.8 Hz, 2H), 3.29 (m, 1H), 2.98 (bs, 4H), 2.65-2.52 (m, 4H), 2.23 (bs, 3H), 1.29 (d, J=6.7 Hz, 6H); ¹³C NMR (126 MHz, DMSO) δ 156.8, 151.7, 151.6, 137.3, 136.9, 133.6, 126.4, 124.9, 120.3, 118.3, 112.9, 111.7, 106.4, 102.1, 70.9, 60.2, 55.2, 52.0, 50.5, 49.1, 16.6. HRMS (ESI+): m/z calcd for C₂₈H₃₆N₇O₄S (M+H)⁺ 566.2544, found 566.2542 m/z calcd for C₂₈H₃₅N₇O₄S Na (M+Na)⁺ 588.2363, found 588.2363; HPLC-MS (ESI+): m/z 566.0 [100% (M+H)⁺], 283.6 [60%, ((M+2H)²⁺/2)].

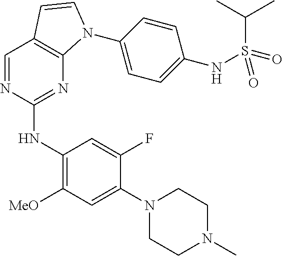 [SMILES: COc1cc(N2CCN(C)CC2)c(F)cc1Nc1ncc2ccn(-c3ccc(NS(=O)(=O)C(C)C)cc3)c2n1]

N-(4-(2-((5-Fluoro-2-methoxy-4-(4-methylpiperazin-1-yl)phenyl)amino)-7H-pyrrolo[2,3-d]pyrimidin-7-yl)phenyl)propane-2-sulfonamide (SR8-143). The pyrrolopyrimidine SR8-143 was obtained as light brown foam (0.030 g, 42% yield) from SR8-138 (0.050 g, 0.142 mmol) and YM2-091 (0.031 g, 0.128 mmol) by following the general procedure A. HPLC: >97% [t_{R}=13.2 min, isocratic 50% MeOH and 50% water (with 0.1% TFA), 20 min]. ¹H NMR (500 MHz, DMSO) δ 9.98 (s, 1H), 8.80 (s, 1H), 8.26 (d, J=15.2 Hz, 1H), 7.83 (m, 1H), 7.82 (d, J=8.9 Hz, 2H), 7.63 (d, J=3.7 Hz, 1H), 7.42 (d, J=8.9 Hz, 2H), 6.69 (m, 2H), 3.88 (s, 3H), 3.29 (m, 1H), 3.01 (m, 4H), 2.48 (m, 4H), 2.24 (s, 3H), 1.29 (d, J=6.9 Hz, 6H); ¹⁹F NMR (471 MHz, DMSO) δ −131.57 (dd, J=15.3, 8.1); HRMS (ESI+): m/z calcd for C₂₇H₃₃FN₇O₃S (M+H)⁺ 554.2344, found 554.2342 m/z calcd for C₂₇H₃₂FN₇O₃S Na (M+Na)⁺ 576.2164, found 576.2157; HPLC-MS (ESI+): m/z 554.0 [100% (M+H)⁺], 277.6 [90%, ((M+2H)²⁺/2)].

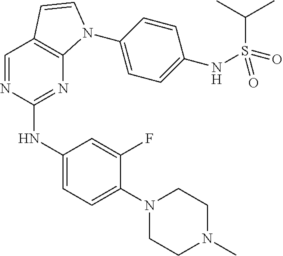 [SMILES: CC(C)S(=O)(=O)Nc1ccc(-n2ccc3cnc(Nc4ccc(N5CCN(C)CC5)c(F)c4)nc32)cc1]

N-(4-(2-((3-Fluoro-4-(4-methylpiperazin-1-yl)phenyl)amino)-7H-pyrrolo[2,3-d]pyrimidin-7-yl)phenyl)propane-2-sulfonamide (SR8-146). The pyrrolopyrimidine SR8-146 was obtained as off-white foam (0.046 g, 68% yield) from SR8-138 (0.050 g, 0.142 mmol) and 3-fluoro-4-(4-methylpiperazin-1-yl)aniline (0.027 g, 0.128 mmol) by following the general procedure A. HPLC: >97% [t_{R}=10.6 min, gradient 5-95% MeOH and water (with 0.1% TFA), 20 min]. ¹H NMR (500 MHz, DMSO) δ 9.97 (s, 1H), 9.55 (s, 1H), 8.80 (s, 1H), 7.95 (dd, J=16.0, 2.5 Hz, 1H), 7.84-7.79 (m, 2H), 7.60 (d, J=3.7 Hz, 1H), 7.48-7.38 (m, 2H), 7.32 (m, 1H), 6.93 (dd, J=10.1, 8.7 Hz, 1H), 6.67 (d, J=3.7 Hz, 1H), 3.31-3.23 (m, 1H), 2.95 (m, 4H), 2.48 (m, 4H), 2.24 (s, 3H), 1.30 (d, J=6.8 Hz, 6H); ¹⁹F NMR (471 MHz, DMSO) δ −122.38 (dd, J=15.8, 10.1 Hz); HRMS (ESI+): m/z calcd for C₂₆H₃₁FN₇O₂S (M+H)⁺ 524.2238, found 524.2236 m/z calcd for C₂₆H₃₀FN₇O₂S Na (M+Na)⁺ 546.2058, found 546.2050; HPLC-MS (ESI+): m/z 524.0 [100% (M+H)⁺], 262.6 [90%, ((M+2H)²⁺/2)].

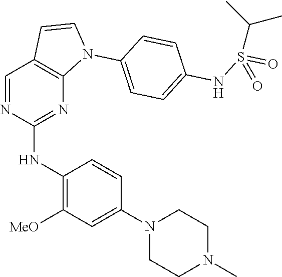 [SMILES: COc1cc(N2CCN(C)CC2)ccc1Nc1ncc2ccn(-c3ccc(NS(=O)(=O)C(C)C)cc3)c2n1]

N-(4-(2-((2-Methoxy-4-(4-methylpiperazin-1-yl)phenyl)amino)-7H-pyrrolo[2,3-d]pyrimidin-7-yl)phenyl)propane-2-sulfonamide (SR8-152). The pyrrolopyrimidine SR8-152 was obtained as light brown foam (0.044 g, 64% yield) from SR8-138 (0.050 g, 0.142 mmol) and 2-methoxy-4-(4-methylpiperazin-1-yl)aniline (0.028 g, 0.128 mmol) by following the general procedure A. HPLC: >99% [t_{R}=16.1 min, gradient 5-95% MeOH and water (with 0.1% TFA), 20 min]. 1H NMR (500 MHz, DMSO) δ 9.84 (s, 1H), 8.64 (s, 1H), 7.90 (d, J=8.8 Hz, 1H), 7.75 (d, J=9.0 Hz, 2H), 7.69 (s, 1H), 7.50 (d, J=3.8 Hz, 1H), 7.31 (d, J=8.9 Hz, 2H), 6.57 (dd, J=8.5, 3.1 Hz, 2H), 6.36

(dd, J=8.8, 2.6 Hz, 1H), 3.76 (s, 3H), 3.20 (sept, J=6.8 Hz, 1H), 3.04 (m, 4H), 2.45 (m, 4H), 2.18 (s, 3H), 1.21 (d, J=6.7 Hz, 6H); ¹³C NMR (126 MHz, DMSO) δ 157.1, 151.8, 151.7, 150.5, 147.8, 137.0, 133.6, 126.0, 124.4, 122.0, 121.6, 120.4, 113.0, 107.2, 102.2, 100.7, 56.1, 55.2, 52.0, 49.4, 46.2, 16.63.

HRMS (ESI+): m/z calcd for C₂₇H₃₄N₇O₃S (M+H)⁺ 536.2438, found 536.2433 m/z calcd for C₂₇H₃₃N₇O₃S Na (M+Na)⁺ 558.2258, found 558.2247; HPLC-MS (ESI+): m/z 536.0 [60% (M+H)⁺], 268.6 [100%, ((M+2H)²⁺/2)].

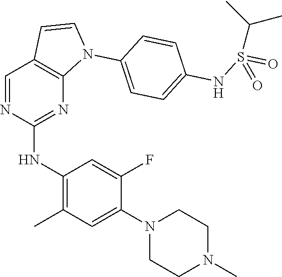 [SMILES: Cc1cc(N2CCN(C)CC2)c(F)cc1Nc1ncc2ccn(-c3ccc(NS(=O)(=O)C(C)C)cc3)c2n1]

N-(4-(2-((5-Fluoro-2-methyl-4-(4-methylpiperazin-1-yl)phenyl)amino)-7H-pyrrolo[2,3-d]pyrimidin-7-yl)phenyl)propane-2-sulfonamide (SR8-153). The pyrrolopyrimidine SR8-153 was obtained as a beige foam (0.034 g, 49% yield) from SR8-138 (0.050 g, 0.142 mmol) and 5-fluoro-2-methyl-4-(4-methylpiperazin-1-yl)aniline (0.029 g, 0.128 mmol) by following the general procedure A. HPLC: >99% [t_{R}=16.5 min, gradient 5-95% MeOH and water (with 0.1% TFA), 20 min]. ¹H NMR (500 MHz, DMSO) δ 9.91 (s, 1H), 8.72 (s, 1H), 8.44 (s, 1H), 7.80 (d, J=8.9 Hz, 2H), 7.65 (d, J=15.0 Hz, 1H), 7.57 (d, J=3.7 Hz, 1H), 7.34 (d, J=8.9 Hz, 2H), 6.84 (d, J=9.8 Hz, 1H), 6.63 (d, J=3.7 Hz, 1H), 3.23 (sept, J=6.8 Hz, 1H), 2.99 (m, 4H), 2.58-2.50 (m, 4H), 2.27 (s, 3H), 2.20 (s, 3H), 1.26 (d, J=6.8 Hz, 6H); HRMS (ESI+): m/z calcd for C₂₇H₃₃FN₇O₂S (M+H)⁺ 538.2395, found 538.2392 m/z calcd for C₂₇H₃₂FN₇O₂S Na (M+Na)⁺ 560.2214, found 560.2205; HPLC-MS (ESI+): m/z 538.0 [100% (M+H)⁺], 269.6 [100%, ((M+2H)²⁺/2)].

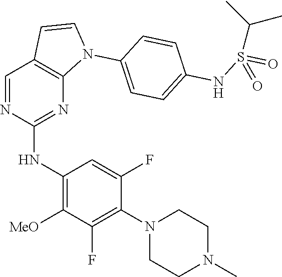 [SMILES: COc1c(Nc2ncc3ccn(-c4ccc(NS(=O)(=O)C(C)C)cc4)c3n2)cc(F)c(N2CCN(C)CC2)c1F]

N-(4-(2-((3,5-Difluoro-2-methoxy-4-(4-methylpiperazin-1-yl)phenyl)amino)-7H-pyrrolo[2,3-d]pyrimidin-7-yl)phenyl)propane-2-sulfonamide (SR8-139). The pyrrolopyrimidine SR8-139 was obtained as a brown foam (0.032 g, 44% yield) from SR8-138 (0.050 g, 0.142 mmol) and 3,5-difluoro-2-methoxy-4-(4-methylpiperazin-1-yl)aniline (0.033 g, 0.128 mmol) by following the general procedure A. HPLC: >99% [t_{R}=4.4 min, isocratic 50% MeOH and water (with 0.1% TFA), 20 min]. ¹H NMR (500 MHz, DMSO) δ 9.99 (s, 1H), 8.84 (s, 1H), 8.26 (s, 1H), 8.14 (dd, J=15.1, 2.0 Hz, 1H), 7.80 (d, J=8.9 Hz, 2H), 7.66 (d, J=3.7 Hz, 1H), 7.42 (d, J=8.9 Hz, 2H), 6.71 (d, J=3.7 Hz, 1H), 3.84 (s, 3H), 3.30-3.24 (m, 1H), 3.07 (m, 4H), 2.42 (m, 4H), 2.22 (s, 3H), 1.29 (d, J=6.8 Hz, 6H); ¹⁹F NMR (471 MHz, DMSO) δ −125.49 (d, J=15.0 Hz), δ −138.20 (d, J=4.0 Hz); HRMS (ESI+): m/z calcd for C₂₇H₃₂F₂N₇O₃S (M+H)⁺ 572.2250, found 572.2239 m/z calcd for C₂₇H₃₁F₂N₇O₃S Na (M+Na)⁺ 594.2060, found 594.2051; HPLC-MS (ESI+): m/z 572.0 [100% (M+H)⁺], 286.6 [60%, ((M+2H)²⁺/2)].

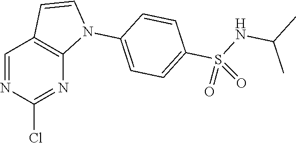 [SMILES: CC(C)NS(=O)(=O)c1ccc(-n2ccc3cnc(Cl)nc32)cc1]

4-(2-Chloro-7H-pyrrolo[2,3-d]pyrimidin-7-yl)-N-isopropylbenzenesulfonamide (SR8-145). The pyrrolopyrimidine SR8-145 was obtained as an off-white solid (0.680 g, 67%) from (2-chloro-7H-(2,3-d)pyrrolopyrimidine (0.449 g, 2.921 mmol) and 4-iodo-N-isopropylbenzenesulfonamide (0.950 g, 2.921 mmol) by following the method described for SR8-127. ¹H NMR (500 MHz, DMSO) δ 9.10 (s, 1H), 8.18 (d, J=3.8 Hz, 1H), 8.10 (d, J=8.8 Hz, 2H), 8.02 (d, J=8.7 Hz, 2H), 7.73 (d, J=7.1 Hz, 1H), 7.00 (d, J=3.8 Hz, 1H), 3.33 (m, 1H), 1.01 (d, J=6.5 Hz, 6H); ¹³C NMR (126 MHz, DMSO) δ 153.5, 152.9, 151.7, 140.6, 139.7, 131.1, 128.3, 124.2, 119.5, 103.1, 45.8, 23.8. HPLC-MS (ESI+): m/z 350.9 [100% (M+H)⁺], (ESI−): m/z 349.0 [100%, (M−1)⁻].

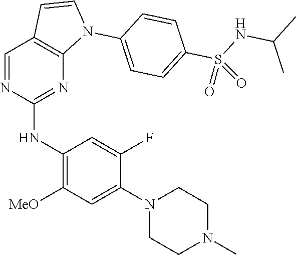 [SMILES: COc1cc(N2CCN(C)CC2)c(F)cc1Nc1ncc2ccn(-c3ccc(S(=O)(=O)NC(C)C)cc3)c2n1]

4-(2-((5-Fluoro-2-methoxy-4-(4-methylpiperazin-1-yl)phenyl)amino)-7H-pyrrolo[2,3-d]pyrimidin-7-yl)-N-isopropylbenzenesulfonamide (SR8-147). The pyrrolopyrimidine SR8-147 was obtained as a brown foam (0.034 g, 49% yield) from SR8-145 (0.050 g, 0.142 mmol) and 5-fluoro-2-methoxy-4-(4-methylpiperazin-1-yl)aniline (0.031 g, 0.128 mmol) by following the general procedure A. HPLC: >99% [t_{R}=18.3 min, gradient 5-95% MeOH and water (with 0.1% TFA), 20 min]. ¹H NMR (500 MHz, DMSO) δ 8.83 (s, 1H), 8.22-8.15 (m, 3H), 7.96 (m, 3H), 7.81 (d, J=3.8 Hz, 1H), 7.72 (d, J=7.0 Hz, 1H), 6.77 (d, J=3.8 Hz, 1H), 6.71 (d, J=8.2 Hz, 1H), 3.88 (s, 3H), 3.30-3.19 (m, 1H), 3.02 (m, 4H), 2.50 (m, 4H), 2.25 (s, 3H), 1.00 (d, J=6.5 Hz, 6H); ¹⁹F NMR (471 MHz, DMSO) δ −131.50 (dd, J=15.1, 8.2 Hz); HRMS (ESI+): m/z calcd for C₂₇H₃₃FN₇O₃S (M+H)⁺ 554.2344, found 554.2339 m/z calcd for C₂₇H₃₂FN₇O₃S Na (M+Na)⁺ 576.2164, found 576.2158; HPLC-MS (ESI+): m/z 554.0 [100% (M+H)⁺], 277.6 [60%, ((M+2H)²⁺/2)].

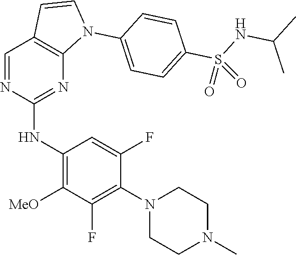 [SMILES: COc1c(Nc2ncc3ccn(-c4ccc(S(=O)(=O)NC(C)C)cc4)c3n2)cc(F)c(N2CCN(C)CC2)c1F]

4-(2-((3,5-Difluoro-2-methoxy-4-(4-methylpiperazin-1-yl)phenyl)amino)-7H-pyrrolo[2,3-d]pyrimidin-7-yl)-N-isopropylbenzenesulfonamide (SR8-148). The pyrrolopyrimidine SR8-148 was obtained as a brown foam (0.030 g, 41% yield) from SR8-145 (0.050 g, 0.142 mmol) and 3,5-difluoro-2-methoxy-4-(4-methylpiperazin-1-yl)aniline (0.033 g, 0.128 mmol) by following the general procedure A. HPLC: >97% [t_{R}=8.3 min, isocratic 55% MeOH and 45% water (with 0.1% TFA), 20 min]. ¹H NMR (500 MHz, DMSO) δ 8.88 (s, 1H), 8.41 (s, 1H), 8.19 (d, J=8.8 Hz, 2H), 8.10 (dd, J=14.9, 2.0 Hz, 1H), 7.96 (d, J=8.8 Hz, 2H), 7.85 (d, J=3.8 Hz, 1H), 7.74 (d, J=7.1 Hz, 1H), 6.80 (d, J=3.8 Hz, 1H), 3.85 (s, 3H), 3.29 (m, 1H), 3.09 (m, 4H), 2.43 (m, 4H), 2.23 (s, 3H), 1.01 (d, J=6.5 Hz, 6H); ¹⁹F NMR (471 MHz, DMSO) δ −125.40 (d, J=14.8 Hz), −138.02. HRMS (ESI+): m/z calcd for C₂₇H₃₂F₂N₇O₃S (M+H)⁺ 572.2250, found 572.2247 m/z calcd for C₂₇H₃₁F₂N₇O₃S Na (M+Na)⁺ 594.2069, found 594.2062; HPLC-MS (ESI+): m/z 572.0 [100% (M+H)⁺], 286.6 [40%, ((M+2H)²⁺/2)].

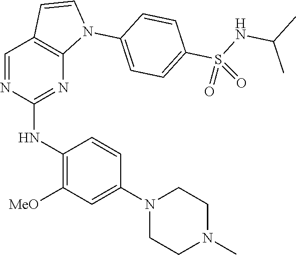 [SMILES: COc1cc(N2CCN(C)CC2)ccc1Nc1ncc2ccn(-c3ccc(S(=O)(=O)NC(C)C)cc3)c2n1]

N-isoPropyl-4-(2-((2-methoxy-4-(4-methylpiperazin-1-yl)phenyl)amino)-7H-pyrrolo[2,3-d]pyrimidin-7-yl)benzenesulfonamide (SR8-149). The pyrrolopyrimidine SR8-149 was obtained as a brown foam (0.032 g, 47% yield) from SR8-145 (0.050 g, 0.142 mmol) and 2-methoxy-4-(4-methylpiperazin-1-yl)aniline (0.028 g, 0.128 mmol) by following the general procedure A. HPLC: >97% [t_{R}=17.3 min, gradient 5-95% MeOH and water (with 0.1% TFA), 20 min]. ¹H NMR (500 MHz, DMSO) δ 8.67 (s, 1H), 8.11 (d, J=8.8 Hz, 2H), 7.87 (d, J=8.8 Hz, 2H), 7.84 (m, 2H), 7.68 (d, J=3.8 Hz, 1H), 7.62 (d, J=7.1 Hz, 1H), 6.64 (d, J=3.8 Hz, 1H), 6.59 (d, J=2.6 Hz, 1H), 6.41 (dd, J=8.9, 2.6 Hz, 1H), 3.76 (s, 3H), 3.21 (d, J=6.6 Hz, 1H), 3.05 (m, 4H), 2.40 (m, 4H), 2.17 (s, 3H), 0.93 (d, J=6.5 Hz, 6H); ¹³C NMR (126 MHz, DMSO) δ 157.3, 152.3, 152.1, 150.9, 148.1, 140.8, 139.1, 128.0, 125.4, 23.0, 121.9, 121.6, 113.2, 107.2, 103.4, 100.7, 56.1, 55.2, 49.4, 46.3, 45.8, 23.7. HRMS (ESI+): m/z calcd for C₂₇H₃₄N₇O₃S (M+H)⁺ 536.2438, found 536.2431 m/z calcd for C₂₇H₃₃N₇O₃S Na (M+Na)⁺ 558.2258, found 558.2251; HPLC-MS (ESI+): m/z 536.0 [100% (M+H)⁺], 268.6 [90%, ((M+2H)²⁺/2)].

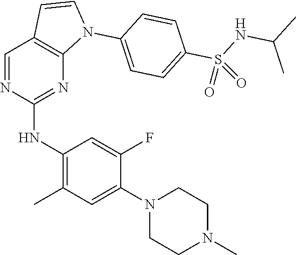 [SMILES: Cc1cc(N2CCN(C)CC2)c(F)cc1Nc1ncc2ccn(-c3ccc(S(=O)(=O)NC(C)C)cc3)c2n1]

4-(2-((5-Fluoro-2-methyl-4-(4-methylpiperazin-1-yl)phenyl)amino)-7H-pyrrolo[2,3-d]pyrimidin-7-yl)-N-isopropylbenzenesulfonamide (SR8-154). The pyrrolopyrimidine SR8-154 was obtained as a beige foam (0.027 g, 40% yield) from SR8-145 (0.050 g, 0.142 mmol) and 5-fluoro-2-methyl-4-(4-methylpiperazin-1-yl)aniline (0.029 g, 0.128 mmol) by following the general procedure A. HPLC: >98% [t_{R}=17.2 min, gradient 5-95% MeOH and water (with 0.1% TFA), 20 min]. ¹H NMR (500 MHz, DMSO) δ 8.76 (s, 1H), 8.60 (s, 1H), 8.18 (d, J=8.8 Hz, 2H), 7.88 (d, J=8.8 Hz, 2H), 7.77 (d, J=3.8 Hz, 1H), 7.70 (d, J=7.1 Hz, 1H), 7.61 (d, J=14.9 Hz, 1H), 6.86 (d, J=9.9 Hz, 1H), 6.72 (d, J=3.8 Hz, 1H), 3.30-3.23 (m, 1H), 2.99 (d, J=5.5 Hz, 5H), 2.51-2.41 (m, 17H), 2.24 (s, 4H), 2.21 (s, 4H), 0.99 (d, J=6.0 Hz, 6H); ¹⁹F NMR (471 MHz, DMSO) δ −126.65 (dd, J=14.8, 9.7 Hz); HRMS (ESI+): m/z calcd for C₂₇H₃₃FN₇O₃S (M+H)⁺ 538.2395, found 538.2389 m/z calcd for C₂₇H₃₂FN₇O₃Na (M+Na)⁺ 560.2214, found 560.2207; HPLC-MS (ESI+): m/z 538.1 [100% (M+H)⁺], 269.6 [60%, ((M+2H)²⁺/2)].

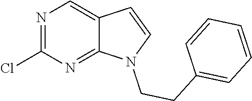 [SMILES: Clc1ncc2ccn(CCc3ccccc3)c2n1]

2-Chloro-7-phenethyl-7H-pyrrolo[2,3-d]pyrimidine (AM1-023): 2-Chloro-7H-pyrrolo[2,3-d]pyrimidine (1.0 g, 0.0065 mol), (2-bromoethyl)benzene (1.3 g 0.0072 mol), and potassium carbonate (0.9 g, 0.0065 mol) were dissolved in DMF (7 mL). The solution was left to stir at 80° C. for 24 hours, after which the solution was diluted with ethyl acetate (70 mL), washed with deionized water (35 mL×3), and washed with brine (35 mL). The organic layer was dried over sodium sulfate and concentrated under reduced pressure. The crude material was purified using gradient 50-100% ethyl acetate/hexane to yield the brown solid (1.2 g 71%). ¹H NMR (500 MHz, DMSO) δ 8.89 (s, 1H), 7.58 (d, J=3.6 Hz, 1H), 7.27-7.11 (m, 5H), 6.63 (d, J=3.6 Hz, 1H), 4.47 (dd, J=7.7, 6.7 Hz, 2H), 3.12 (t, J=7.2 Hz, 2H). HPLC-MS (ESI+) m/z, 258.1 (M+H)⁺.

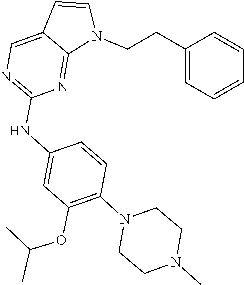 [SMILES: CC(C)Oc1cc(Nc2ncc3ccn(CCc4ccccc4)c3n2)ccc1N1CCN(C)CC1]

N-(3-isopropoxy-4-(4-methylpiperazin-1-yl)phenyl)-7-phenethyl-7H-pyrrolo[2,3-d]pyrimidin-2-amine (AM1-025): 2-chloro-7-phenethyl-7H-pyrrolo[2,3-d]pyrimidine (0.050 g, 0.194 mmol), 3-isopropoxy-4-(4-methylpiperazin-1-yl)aniline (0.044 g, 0.175 mmol), BINAP (0.012 g, 0.0194 mmol), and cesium carbonate (0.095 g, 0.291 mmol) were dissolved in 1,4 dioxane (2 mL). The solution was purged with argon, palladium acetate (0.0044 g, 0.0194 mmol) was added, and the solution was stirred at 105° C. for 24 hours. The solution was diluted with ethyl acetate (30 mL), washed with sodium bicarbonate (2×35 mL) and then brine (20 mL). The organic layer was dried over sodium sulfate and concentrated under reduced pressure. Purified by gradient DCM/MeOH 0%-15% to yield the desired product as a tan solid (0.070 g, 85%). ¹H NMR (500 MHz, DMSO) δ 9.18 (s, 1H), 8.64 (s, 1H), 7.68 (d, J=2.4 Hz, 1H), 7.33-7.22 (m, 7H), 6.82 (d, J=8.7 Hz, 1H), 6.37 (d, J=3.5 Hz, 1H), 4.54 (hept, J=6.0 Hz, 1H), 4.42-4.35 (m, 2H), 3.14 (dd, J=8.4, 6.7 Hz, 2H), 2.28 (s, 3H), 1.24 (d, J=6.0 Hz, 6H). HPLC-MS (ESI+) m/z, 471.3 (M+H)⁺; HRMS (ESI+) m/z calculated for C₂₈H₃₄N₆O (M) 470.2785, found 470.2794.

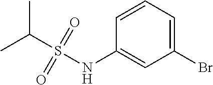

N-(3-Bromophenyl)propane-2-sulfonamide (AM1-034): Bromoaniline (1.0 g, 0.0058 mol) and sulfonyl isopropyl chloride (0.969 g, 0.0068 mol) were dissolved in pyridine (10 mL). The solution stirred overnight at 50° C. The solution was diluted with ethyl acetate (30 mL), washed with 1M HCl (2×15 mL), and brine (20 mL). The organic layer was dried over sodium sulfate and evaporated under reduced pressure to yield a tan solid (0.526 g, 32%). ¹H NMR (500 MHz, DMSO) δ 10.00 (s, 1H), 7.40 (t, 1H), 7.32-7.21 (m, 3H), 3.32-3.23 (m, 1H), 1.25 (d, J=6.8 Hz, 6H).

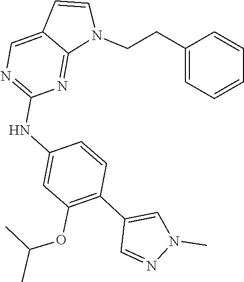 [SMILES: CC(C)Oc1cc(Nc2ncc3ccn(CCc4ccccc4)c3n2)ccc1-c1cnn(C)c1]

N-(3-isopropoxy-4-(1-methyl-1H-pyrazol-4-yl)phenyl)-7-phenethyl-7H-pyrrolo[2,3-d]pyrimidin-2-amine (AM1-038): 2-Chloro-7-phenethyl-7H-pyrrolo[2,3-d]pyrimidine (0.050 g, 0.194 mmol), 3-isopropoxy-4-(1-methyl-1H-pyrazol-4-yl)aniline (0.045 g, 0.194 mmol), BINAP (0.012 g, 0.0194 mmol), and cesium carbonate (0.095 g, 0.291 mmol) were dissolved in 1,4-dioxane (2 mL). The solution was purged with argon, palladium acetate (0.0044 g, 0.0194 mmol) was added, and the solution was stirred at 105° C. for 24 hours. The solution was diluted with ethyl acetate (30 mL), washed with sodium bicarbonate (2×25 mL) and then brine (20 mL). The organic layer was dried over sodium sulfate and concentrated under reduced pressure. Purified by gradient chromatography (EtOAc/hexanes 20%-50% then DCM/MeOH 0%-15%). Further purification by trituration with ethyl acetate/hexanes to yield white solid product to yield the desired product as a white solid (0.021 g, 23%). ¹H NMR (500 MHz, DMSO) δ 9.40 (s, 1H), 8.68 (s, 1H), 7.97 (s, 1H), 7.91 (d, J=2.2 Hz, 1H), 7.83 (d, J=0.7 Hz, 1H), 7.44 (d, J=8.4 Hz, 1H), 7.34-7.26 (m, 3H), 7.25-7.17 (m, 4H), 6.40 (d, J=3.6 Hz, 1H), 4.62 (hept, J=6.0 Hz, 1H), 4.43 (dd, J=8.2, 6.8 Hz, 2H), 3.86 (s, 3H), 3.16 (t, J=7.5 Hz, 2H), 1.31 (d, J=6.0 Hz, 6H). HPLC-MS (ESI+) m/z, 453.3 (M+H)⁺; HRMS (ESI+) m/z calculated for C₂₈H₃₄N₆O (M) 452.2322, found 452.2325.

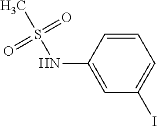

N-(3-Iodophenyl)methanesulfonamide (AM1-051): 3-iodoaniline (0.1 g, 0.456 mmol), pyridine (0.108 g, 1.368 mmol), and methanesulfonyl chloride (0.078 g, 0.684 mmol) were dissolved in dry DCM (2 mL). The solution stirred overnight at room temperature. The solution was diluted with ethyl acetate (30 mL), washed with 1M HCl (2×15 mL), and brine (20 mL). The organic layer was dried over sodium sulfate and evaporated under reduced pressure to yield a tan solid (0.116 g, 85%). ¹H NMR (600 MHz, DMSO) δ 9.90 (s, 1H), 7.55 (t, J=2.0 Hz, 1H), 7.46 (dt, J=7.7, 1.3 Hz, 1H), 7.26-7.22 (m, 1H), 7.14 (t, J=8.0 Hz, 1H), 3.02 (s, 3H). HPLC-MS (ESI+) m/z, 296.0 (M−H)⁻.

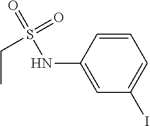

N-(3-Iodophenyl)ethanesulfonamide (AM1-053): 3-iodoaniline (0.1 g, 0.456 mmol), pyridine (0.108 g, 1.368 mmol), and ethanesulfonyl chloride (0.087 g, 0.684 mmol) were dissolved in dry DCM (2 mL). The solution stirred overnight at 50° C. The solution was diluted with ethyl acetate (30 mL), washed with 1M HCl (2×15 mL), and brine (20 mL). The organic layer was dried over sodium sulfate and evaporated under reduced pressure to yield a brown solid (0.158 g, 108%). ¹H NMR (600 MHz, DMSO) δ 9.94 (s, 1H), 7.56 (t, J=1.9 Hz, 1H), 7.44 (d, J=7.8 Hz, 1H), 7.24 (dd, J=8.1, 2.2 Hz, 1H), 7.12 (t, J=8.0 Hz, 1H), 3.13 (q, J=7.3 Hz, 2H), 1.19 (t, J=7.3 Hz, 3H). HPLC-MS (ESI+) m/z, 310.0 (M−H)⁻.

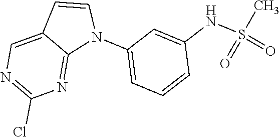 [SMILES: CS(=O)(=O)Nc1cccc(-n2ccc3cnc(Cl)nc32)c1]

N-(3-(2-Chloro-7H-pyrrolo[2,3-d]pyrimidin-7-yl)phenyl)methanesulfonamide (AM1-055): N-(3-iodophenyl)methanesulfonamide (0.050 g 0.168 mmol), 2-chloro-7H-pyrrolo[2,3-d]pyrimidine (0.020 g, 0.129 mmol), copper iodide (0.0025 g, 0.0129 mmol), (1R,2R)-cyclohexane-1,2-diamine (0.0015 g, 0.0129 mmol) and potassium phosphate (0.082 g, 0.387 mmol) were dissolved in 1,4 dioxane (2 mL). The solution was stirred at 100° C. for 24 hours. (1R,2R)-cyclohexane-1,2-diamine (5 uL) was added and reaction was stirred for 24 hours at 150° C. The solution was diluted with 30 mL of ethyl acetate, washed with ammonium chloride (2×15 mL), and brine (2×15 mL). The organic layer was dried over sodium sulfate and evaporated under reduced pressure. Purified by chromatography (hexane/EtOAc 30%-100% then DCM/MeOH 0%-5% to yield tan solid product (0.010 g, 24%). HPLC-MS (ESI+) m/z, 323.1 (M+H)⁺.

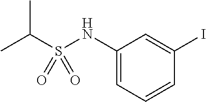

N-(3-Iodophenyl)propane-2-sulfonamide (AM1-061): 3-Iodoaniline (1.0 g, 0.0046 mol) and pyridine (0.433 g, 0.0055 mol) were dissolved in DCM (22 mL, 0.2M). The solution was brought to 0° C., purged with argon, and propane-2-sulfonyl chloride (0.65 g, 0.0046 mol) was added to the solution. The solution was gradually brought to room temperature and stirred overnight under argon. The solution was quenched with water (1 mL) and stirred for 30 minutes. After which, the solution was diluted with DCM (30 mL), washed with 1M HCl (20 mL), sodium bicarbonate (20 mL), and brine (20 mL). The organic layer was dried over sodium sulfate and evaporated under reduced pressure to yield a red solid (1.445 g, 97%). ¹H NMR (500 MHz, DMSO) δ 9.91 (s, 1H), 7.57 (t, J=1.9 Hz, 1H), 7.43 (dt, J=7.8, 1.2 Hz, 1H), 7.26 (dd, J=8.2, 2.2 Hz, 1H), 7.11 (t, J=8.0 Hz, 1H), 3.26 (h, J=6.8 Hz, 1H), 1.24 (d, J=6.7 Hz, 6H). HPLC-MS (ESI+) m/z, 348.1 (M+Na)⁺.

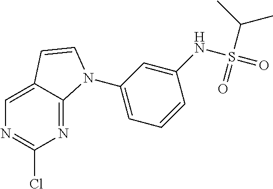 [SMILES: CC(C)S(=O)(=O)Nc1cccc(-n2ccc3cnc(Cl)nc32)c1]

N-(3-(2-Chloro-7H-pyrrolo[2,3-d]pyrimidin-7-yl)phenyl)propane-2-sulfonamide (AM1-063-B2): N-(3-Iodophenyl)propane-2-sulfonamide (1.27 g, 3.907 mmol), 2-chloro-7H-pyrrolo[2,3-d]pyrimidine (0.500 g, 3.256 mmol), copper iodide (0.124 g, 1.628 mmol) and potassium phosphate (2.073 g, 9.768 mmol) were dissolved with 1,4-dioxane (16 mL, 0.2M). (1R,2R)-cyclohexane-1,2-diamine (0.186 g, 1.628 mmol) was added. The solution was stirred at 120° C. for 24 hours. The solution was diluted with 75 mL of ethyl acetate, washed with ammonium chloride (2×30 mL), and brine (30 mL). The organic layer was dried over sodium sulfate and evaporated under reduced pressure. Purified by chromatography (hexane/EtOAc 30%-100% then DCM/MeOH 0%-10%) to yield tan solid product (0.592 g, 51%). ¹H NMR (600 MHz, DMSO) δ 10.13 (s, 1H), 9.07 (s, 1H), 8.02 (d, J=3.7 Hz, 1H), 7.74 (t, J=2.2 Hz, 1H), 7.53 (t, J=8.1 Hz, 1H), 7.43 (dd, J=7.9, 2.1 Hz, 1H), 7.27 (dd, J=8.3, 2.2 Hz, 1H), 6.94 (d, J=3.7 Hz, 1H), 3.45 (p, J=6.8 Hz, 1H), 1.31 (d, J=6.8 Hz, 6H). HPLC-MS (ESI+) m/z, 351.1 (M+H)⁺.

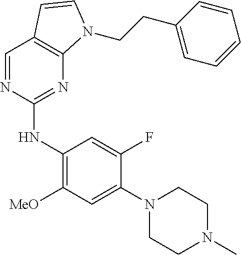 [SMILES: COc1cc(N2CCN(C)CC2)c(F)cc1Nc1ncc2ccn(CCc3ccccc3)c2n1]

N-(5-Fluoro-2-methoxy-4-(4-methylpiperazin-1-yl)phenyl)-7-phenethyl-7H-pyrrolo[2,3-d]pyrimidin-2-amine (AM1-079): 2-Chloro-7-phenethyl-7H-pyrrolo[2,3-d]pyrimidine (0.050 g, 0.194 mmol), 5-fluoro-2-methoxy-4-(4-methylpiperazin-1-yl)aniline (0.046 g, 0.194 mmol), BINAP (0.012 g, 0.0194 mmol), and cesium carbonate (0.095 g, 0.291 mmol) were dissolved in 1,4 dioxane (2 mL, 0.097M). The flask was capped and purged with argon for ten minutes. Palladium acetate (0.0044 g, 0.0194 mmol) was added, and the solution was stirred at 105° C. for 18 hours. The solution was diluted with ethyl acetate (30 mL), washed with sodium bicarbonate (20 mL), and brine (15 mL). The organic layer was dried over sodium sulfate and concentrated under reduced pressure. Purified by gradient DCM/MeOH 0%-20% to yield the desired product as a brown solid (0.033 g, 37%). ¹H NMR (500 MHz, DMSO) δ 8.67 (s, 1H), 8.37 (d, J=15.1 Hz, 1H), 7.68 (d, J=1.3 Hz, 1H), 7.29-7.16 (m, 6H), 6.73 (d, J=8.2 Hz, 1H), 6.42 (d, J=3.6 Hz, 1H), 4.40-4.34 (m, 2H), 3.90 (s, 3H), 3.12 (t, J=7.5 Hz, 2H), 3.04 (d, J=5.1 Hz, 5H), 2.27 (s, 4H), 1.24 (s, 1H). HPLC-MS (ESI+) m/z, 461.3 (M+H)⁺; HRMS (ESI+) m/z calculated for C₂₆H₂₉FN₆O (M) 461.2460, found 461.2457.

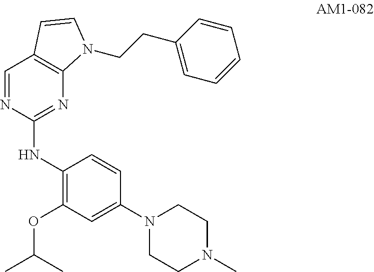 [SMILES: CC(C)Oc1cc(N2CCN(C)CC2)ccc1Nc1ncc2ccn(CCc3ccccc3)c2n1]

N-(2-isoPropoxy-4-(4-methylpiperazin-1-yl)phenyl)-7-phenethyl-7H-pyrrolo[2,3-d]pyrimidin-2-amine (AM1-082): 2-Chloro-7-phenethyl-7H-pyrrolo[2,3-d]pyrimidine (0.050 g, 0.194 mmol), 2-isopropoxy-4-(4-methylpiperazin-1-yl)aniline (0.048 g, 0.194 mmol), BINAP (0.012 g, 0.0194 mmol), and cesium carbonate (0.095 g, 0.291 mmol) were dissolved in 1,4 dioxane (2 mL, 0.097M). The solution was capped and purged with argon for ten minutes. Palladium acetate (0.0044 g, 0.0194 mmol) was added, and the solution was stirred at 105° C. for 18 hours. The solution was diluted with ethyl acetate (30 mL) and washed with sodium bicarbonate (15 mL) and then brine (15 mL). The organic layer was dried over sodium sulfate and concentrated under reduced pressure. Purified by gradient hexane/EtOAc 70%-100% then DCM/MeOH 0%-15% to yield the desired product as a yellow oil (0.036 g, 40%). ¹H NMR (500 MHz, DMSO) δ 8.61 (s, 1H), 8.31 (d, J=8.8 Hz, 1H), 7.51 (s, 1H), 7.31-7.24 (m, 2H), 7.24-7.14 (m, 4H), 6.69 (d, J=2.6 Hz, 1H), 6.54 (dd, J=8.9, 2.6 Hz, 1H), 6.38 (d, J=3.5 Hz, 1H), 4.70 (hept, J=6.0 Hz, 1H), 4.36 (dd, J=8.2, 6.6 Hz, 2H), 3.16-3.08 (m, 6H), 2.26 (s, 3H), 1.32 (d, J=6.1 Hz, 7H). HPLC-MS (ESI+) m/z, 471.3 (M+H)⁺; HRMS (ESI+) m/z calculated for C₂₈H₃₄N₆O (M) 471.2867, found 471.2864.

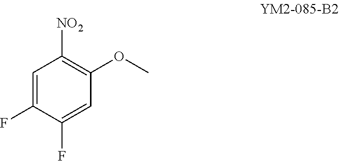

1,2-Difluoro-4-methoxy-5-nitrobenzene (YM2-085-B2): 2,5-Difluoro-6-nitrophenol (2.0 g, 0.011 mol) was dissolved in DMF (16 mL). Potassium carbonate (2.21 g, 0.016 mol) was slowly added, followed by methyl iodide (2.02 g, 0.014 mol). The solution was left to stir at room temperature for 18 hours, after which the solution was quenched with water (50 mL) and extracted with ethyl acetate (3×50 mL). The organic layers were combined, dried over sodium sulfate, and concentrated under reduced pressure to yield the desired product as a yellow oil (1.96 g, 90%). ¹H NMR (500 MHz, DMSO) δ 8.24 (dd, J=10.2, 8.4 Hz, 1H), 7.64 (dd, J=12.6, 6.8 Hz, 1H), 3.94 (s, 3H).

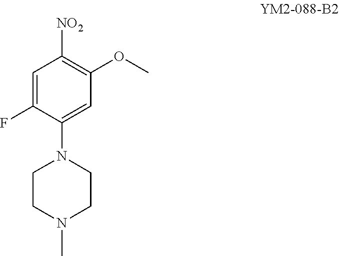 [SMILES: COc1cc(N2CCN(C)CC2)c(F)cc1[N+](=O)[O-]]

1-(2-Fluoro-5-methoxy-4-nitrophenyl)-4-methylpiperazine (YM2-088-B2): 1,2-difluoro-4-methoxy-5-nitrobenzene (1.94 g, 0.010 mol) was dissolved in acetonitrile (29 mL). 1-methylpiperazine (1.13 g, 0.011 mol) was added, and the solution stirred at 80 C for 18 hours. The solution was concentrated under reduced pressure then diluted with ethyl acetate (60 mL) and washed with water (40 mL). The aqueous layer was re-extracted with ethyl acetate (6×40 mL) and the organic layers were combined, dried over sodium sulfate, and concentrated under reduced pressure. Purified by gradient DCM/MeOH 0-15% to yield the desired product as a yellow solid (2.522 g, 91%). ¹H NMR (500 MHz, DMSO) δ 7.85 (d, J=13.6 Hz, 1H), 6.68 (d, J=7.6 Hz, 1H), 3.94 (s, 3H), 2.26 (s, 3H). HPLC-MS (ESI+) m/z, 270.2 (M+H)⁺.

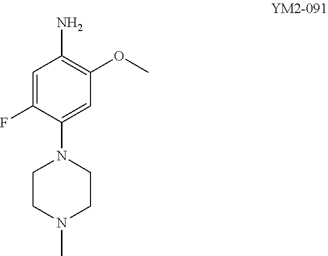 [SMILES: COc1cc(N2CCN(C)CC2)c(F)cc1N]

5-Fluoro-2-methoxy-4-(4-methylpiperazin-1-yl)aniline (YM2-091): Palladium on carbon (0.371 g, 0.0037) was added to a flask and purged with argon. Methanol (10 mL) was added and the solution was degassed for 10 minutes. 1-(2-fluoro-5-methoxy-4-nitrophenyl)-4-methylpiperazine (1 g, 0.0037 mol) was dissolved in methanol (10 mL) and added to the flask. The solution was evacuated and filled with argon (4×). A hydrogen balloon was attached to the reaction vessel. The vessel was evacuated and filled with hydrogen (4×). The hydrogen was bubbled through the solution, the flask was evacuated once more, and the solution was stirred at 50° C. for 3 hours. The solution was filtered through celite and concentrated under reduced pressure to yield the desired product as a dark purple solid (0.839 g, 94%). ¹H NMR (600 MHz, DMSO) δ 6.53 (d, J=8.1 Hz, 1H), 6.42 (d, J=13.6 Hz, 1H), 4.60 (s, 2H), 3.73 (s, 3H), 2.88 (t, J=4.9 Hz, 4H), 2.48 (s, 5H), 2.24 (s, 3H). HPLC-MS (ESI+) m/z, 240.4 (M+H)⁺.

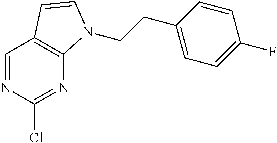 [SMILES: Fc1ccc(CCn2ccc3cnc(Cl)nc32)cc1]

2-Chloro-7-(4-fluorophenethyl)-7H-pyrrolo[2,3-d]pyrimidine (AM1-092): 2-chloro-7H-pyrrolo[2,3-d]pyrimidine (0.3 g, 0.0020 mol), 1-(2-bromoethyl)-4-fluorobenzene (0.436 g, 0.0021 mol), and potassium carbonate (0.27 g, 0.0020 mol) were dissolved in DMF (2.7 mL, 0.7M). The solution was stirred at 80° C. for 24 hours. The solution was diluted with ethyl acetate (40 mL), washed with deionized water (20 mL×3), and washed with brine (20 mL). The organic layer was dried over sodium sulfate and concentrated under reduced pressure. The crude material was purified using gradient 40-100% ethyl acetate/hexane to yield the light pink solid (0.363 g, 67%). ¹H NMR (600 MHz, DMSO) δ 8.88 (s, 1H), 7.57 (d, J=3.6 Hz, 1H), 7.17-7.12 (m, 3H), 7.07-7.00 (m, 2H), 6.64 (d, J=3.6 Hz, 1H), 4.45 (t, J=7.1 Hz, 2H), 3.11 (t, J=7.1 Hz, 3H). HPLC-MS (ESI+) m/z, 275.9 (M+H)⁺.

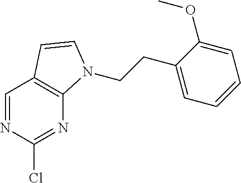 [SMILES: COc1ccccc1CCn1ccc2cnc(Cl)nc21]

2-Chloro-7-(2-methoxyphenethyl)-7H-pyrrolo[2,3-d]pyrimidine (AM1-093): 2-chloro-7H-pyrrolo[2,3-d]pyrimidine (0.3 g, 0.0020 mol), 1-(2-bromoethyl)-2-methoxybenzene (0.462 g 0.0021 mol), and potassium carbonate (0.27 g, 0.0020 mol) were dissolved in DMF (2.7 mL, 0.7M). The solution was left to stir at 80° C. for 24 hours. The solution was diluted with ethyl acetate (40 mL), washed with deionized water (20 mL×3), and washed with brine (20 mL). The organic layer was dried over sodium sulfate and concentrated under reduced pressure. The crude material was purified using gradient 0-100% ethyl acetate/hexane to yield a clear oil (0.246 g, 43%). ¹H NMR (500 MHz, DMSO) δ 8.86 (s, 1H), 7.48 (d, J=3.6 Hz, 1H), 7.19-7.11 (m, 1H), 6.94-6.88 (m, 2H), 6.73 (td, J=7.4, 1.1 Hz, 1H), 6.60 (d, J=3.5 Hz, 1H), 4.45 (t, J=6.9 Hz, 2H), 3.72 (s, 4H), 3.06 (t, J=6.9 Hz, 2H). HPLC-MS (ESI+) m/z, 288.1 (M+H)⁺.

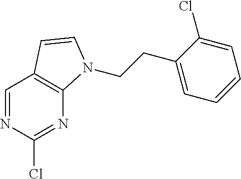 [SMILES: Clc1ncc2ccn(CCc3ccccc3Cl)c2n1]

2-Chloro-7-(2-chlorophenethyl)-7H-pyrrolo[2,3-d]pyrimidine (AM1-094): 2-Chloro-7H-pyrrolo[2,3-d]pyrimidine (0.3 g, 0.0020 mol), 1-(2-bromoethyl)-2-chlorobenzene (0.47 g 0.0021 mol), and potassium carbonate (0.27 g, 0.0020 mol) were dissolved in DMF (2.7 mL, 0.7M). The solution was left to stir at 80° C. for 24 hours. The solution was diluted with ethyl acetate (40 mL), washed with deionized water (20 mL×3), and washed with brine (20 mL). The organic layer was dried over sodium sulfate and concentrated under reduced pressure. The crude material was purified using gradient 0-100% ethyl acetate/hexane to yield the light pink solid (0.145, 25%). ¹H NMR (500 MHz, DMSO) δ 8.88 (s, 1H), 7.53 (d, J=3.6 Hz, 1H), 7.39 (dd, J=7.9, 1.3 Hz, 1H), 7.27-7.09 (m, 3H), 6.63 (d, J=3.5 Hz, 1H), 4.51 (t, J=6.9 Hz, 2H), 3.24 (t, J=6.9 Hz, 2H). HPLC-MS (ESI+) m/z, 292.1 (M+H)⁺.

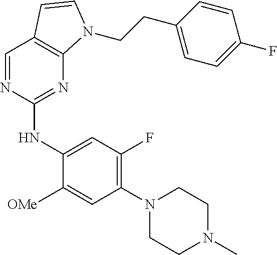 [SMILES: COc1cc(N2CCN(C)CC2)c(F)cc1Nc1ncc2ccn(CCc3ccc(F)cc3)c2n1]

N-(5-Fluoro-2-methoxy-4-(4-methylpiperazin-1-yl)phenyl)-7-(4-fluorophenethyl)-7H-pyrrolo[2,3-d]pyrimidin-2-amine (AM1-107): 2-Chloro-7-(4-fluorophenethyl)-7H-pyrrolo[2,3-d]pyrimidine (0.050 g, 0.181 mmol), 5-fluoro-2-methoxy-4-(4-methylpiperazin-1-yl)aniline (0.039 g, 0.163 mmol), BINAP (0.011 g, 0.0181 mmol), and cesium carbonate (0.088 g, 0.272 mmol) were dissolved in 1,4 dioxane (2 mL, 0.08M). The flask was capped and purged with argon for ten minutes. Palladium acetate (0.0041 g, 0.0181 mmol) was added, and the solution was stirred at 105° C. for 18 hours. The solution was diluted with ethyl acetate (30 mL), washed with sodium bicarbonate (20 mL×3), and brine (15 mL×2). The organic layer was dried over sodium sulfate and concentrated under reduced pressure. Purified by gradient EtOAc/hexanes 70%-100% then DCM/MeOH 0%-15% to yield the desired product as a brown solid (0.062 g, 79%). ¹H NMR (500 MHz, DMSO) δ 8.67 (s, 1H), 8.34 (d, J=15.0 Hz, 1H), 7.67 (s, 1H), 7.27-7.18 (m, 3H), 7.09-7.01 (m, 2H), 6.73 (d, J=8.2 Hz, 1H), 6.42 (d, J=3.5 Hz, 1H), 4.39-4.33 (m, 2H), 3.90 (s, 3H), 3.12 (t, J=7.3 Hz, 2H), 3.06 (s, 4H), 2.60 (s, 5H), 2.34 (s, 4H). HPLC-MS (ESI+) m/z, 479.3 (M+H)⁺; HRMS (ESI+) m/z calculated for C₂₆H₂₈F₂N₆O (M) 479.5553, found 479.2360.

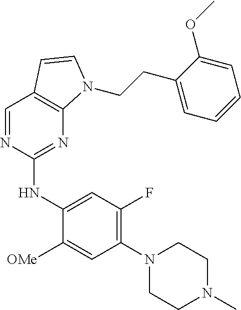 [SMILES: COc1ccccc1CCn1ccc2cnc(Nc3cc(F)c(N4CCN(C)CC4)cc3OC)nc21]

N-(5-Fluoro-2-methoxy-4-(4-methylpiperazin-1-yl)phenyl)-7-(2-methoxyphenethyl)-7H-pyrrolo[2,3-d]pyrimidin-2-amine (AM1-116): 2-chloro-7-(2-methoxyphenethyl)-7H-pyrrolo[2,3-d]pyrimidine (0.050 g, 0.174 mmol), 5-fluoro-2-methoxy-4-(4-methylpiperazin-1-yl)aniline (0.037 g, 0.156 mmol), BINAP (0.011 g, 0.0174 mmol), and cesium carbonate (0.085 g, 0.261 mmol) were dissolved in 1,4 dioxane (2 mL, 0.097 M). The flask was capped and purged with argon for ten minutes. Palladium acetate (0.004 g, 0.0174 mmol) was added, and the solution was stirred at 105° C. for 18 hours. The solution was diluted with ethyl acetate (30 mL), washed with sodium bicarbonate (20 mL×3), and brine (20 mL×3). The organic layer was dried over sodium sulfate and concentrated under reduced pressure. Purified by gradient chromatography (EtOAc/hexanes 70%-100% then DCM/MeOH 0%-20%) to yield the desired product as a tan solid (0.044 g, 52%). ¹H NMR (500 MHz, DMSO) δ 8.65 (s, 1H), 8.36 (d, J=15.0 Hz, 1H), 7.65 (d, J=1.3 Hz, 1H), 7.21 (d, J=3.6 Hz, 1H), 7.17 (td, J=7.8, 1.7 Hz, 1H), 7.03 (dd, J=7.4, 1.7 Hz, 1H), 6.93 (dd, J=8.3, 1.1 Hz, 1H), 6.81-6.70 (m, 2H), 6.41 (d, J=3.5 Hz, 1H), 4.35 (dd, J=8.1, 6.5 Hz, 2H), 3.90 (s, 3H), 3.74 (s, 3H), 3.13-3.03 (m, 7H), 2.29 (s, 3H), 1.92 (s, 1H). HPLC-MS (ESI+) m/z, 491.0 (M+H)⁺; HRMS (ESI+) m/z calculated for C₂₇H₃₁FN₆O₂(M) 491.5909, found 491.2556.

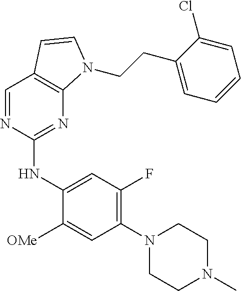 [SMILES: COc1cc(N2CCN(C)CC2)c(F)cc1Nc1ncc2ccn(CCc3ccccc3Cl)c2n1]

7-(2-chlorophenethyl)-N-(5-fluoro-2-methoxy-4-(4-methylpiperazin-1-yl)phenyl)-7H-pyrrolo[2,3-d]pyrimidin-2-amine (AM1-118): 2-chloro-7-(2-chlorophenethyl)-7H-pyrrolo[2,3-d]pyrimidine (0.050 g, 0.171 mmol), 5-fluoro-2-methoxy-4-(4-methylpiperazin-1-yl)aniline (0.037 g, 0.154 mmol), BINAP (0.011 g, 0.0171 mmol), and cesium carbonate (0.084 g, 0.257 mmol) were dissolved in 1,4 dioxane (2 mL, 0.09M). The flask was capped and purged with argon for ten minutes. Palladium acetate (0.004 g, 0.0171 mmol) was added, and the solution was stirred at 105° C. for 18 hours. The solution was diluted with ethyl acetate (30 mL), washed with sodium bicarbonate (20 mL×4), and brine (20 mL×4). The organic layer was dried over sodium sulfate and concentrated under reduced pressure. Purified by gradient EtOAc/hexanes 70%-100% then DCM/MeOH 0%-20% to yield the desired product as a yellow oil (0.010 g, 11%). ¹H NMR (500 MHz, DMSO) δ 8.66 (s, 1H), 8.34 (d, J=14.8 Hz, 1H), 7.64 (d, J=1.2 Hz, 1H), 7.40-7.35 (m, 1H), 7.28 (d, J=3.5 Hz, 1H), 7.27-7.18 (m, 3H), 6.73 (d, J=8.1 Hz, 1H), 6.44 (d, J=3.6 Hz, 1H), 4.42 (dd, J=7.9, 6.5 Hz, 2H), 3.90 (s, 3H), 3.24 (t, J=7.2 Hz, 2H), 3.07 (s, 5H). HPLC-MS (ESI+) m/z, 495.0 (M+H)⁺; HRMS (ESI+) m/z calculated for C₂₆H₂₈ClFN₆O (M) 496.0069, found 495.2059.

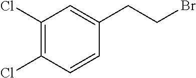 [SMILES: Clc1ccc(CCBr)cc1Cl]

4-(2-Bromoethyl)-1,2-dichlorobenzene (AM1-120): 2-(3,4-Dichlorophenyl)ethan-1-ol (2.0 g, 0.0105 mol), NBS (2.235 g, 0.0126 mol), and PPh₃ (3.294 g, 0.0126 mol) were dissolved in DCM (50 mL, 0.21M) and left to stir for two hours at 0° C. The solution was quenched with water (25 mL) and left to stir for ten minutes. The water was separated, and the organic layer was washed with water (25 mL) and brine (25 mL). The organic layer was dried over sodium sulfate and concentrated under reduced pressure. Purified by column chromatography of hexane to yield a yellow oil (1.642 g, 62%). ¹H NMR (500 MHz, DMSO) δ 7.63-7.55 (m, 2H), 7.31 (dd, J=8.2, 2.1 Hz, 1H), 3.76 (t, J=7.0 Hz, 2H), 3.15 (t, J=7.0 Hz, 2H). HPLC-MS (ESI+) m/z, 279.0 (M+Na)⁺.

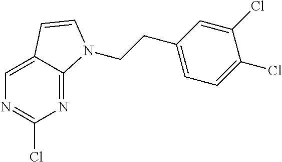 [SMILES: Clc1ncc2ccn(CCc3ccc(Cl)c(Cl)c3)c2n1]

2-Chloro-7-(3,4-dichlorophenethyl)-7H-pyrrolo[2,3-d]pyrimidine (AM1-122): 4-(2-Bromoethyl)-1,2-dichlorobenzene (0.5 g, 0.0020 mol), 2-chloro-7H-pyrrolo[2,3-d]pyrimidine (0.252 g 0.0016 mol), and potassium carbonate (0.227 g, 0.0016 mol) were dissolved in DMF (3 mL). The solution was left to stir at 100° C. for 24 hours. Purified by column chromatography gradient EtOAc/hexanes 0%-80% to yield a yellow oil (0.108 g, 20%). ¹H NMR (500 MHz, DMSO) δ 8.89 (s, 1H), 7.60 (d, J=3.6 Hz, 1H), 7.41 (d, J=2.1 Hz, 1H), 7.06 (dd, J=8.2, 2.1 Hz, 1H), 6.65 (d, J=3.6 Hz, OH), 4.48 (t, J=7.0 Hz, 1H), 3.12 (t, J=6.9 Hz, 1H). HPLC-MS (ESI+) m/z, 325.8 (M+H)⁺.

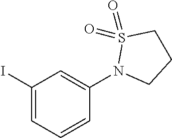 [SMILES: O=S1(=O)CCCN1c1cccc(I)c1]

2-(3-Iodophenyl)isothiazolidine 1,1-dioxide (AM1-124): To a solution of 3-iodoaniline (1.0 g, 4.57 mmol) and Et₃N (1.23 mL, 8.73 mmol) in DCM (9 mL, 0.5 M), 3-chloropropane-1-sulfonyl chloride (0.75 mL, 6.15 mmol) was added. The mixture was stirred for 60 hours at room temperature, diluted with DCM (20 mL), washed with 4N HCl (15 mL×2), and evaporated under reduced pressure. The resulting crude mixture was dissolved in DMF (7 mL, 0.7 M) and DBU (0.8 mL, 5.58 mmol) was added. After being stirred for 3 hours at room temperature, the reaction mixture was poured into 200 mL of hexane/ethyl acetate (1/1) and washed with 4N HCl (100 mL). The organic layer was dried over sodium sulfate and evaporated under reduced pressure to yield an orange oil (1.2 g, 84%). ¹H NMR (500 MHz, DMSO) δ 7.53 (t, 1H), 7.47 (dt, J=7.6, 1.3 Hz, 1H), 7.23 (ddd, J=8.3, 2.3, 1.1 Hz, 1H), 7.17 (t, 1H), 3.75 (t, J=6.5 Hz, 2H), 3.54 (t, J=7.4 Hz, 2H), 2.40 (q, 2H). HPLC-MS (ESI+) m/z, 323.8 (M+H)⁺.

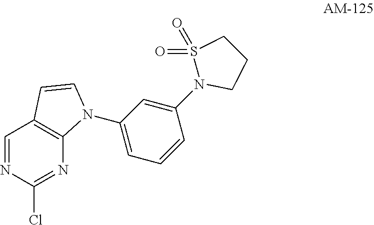 [SMILES: O=S1(=O)CCCN1c1cccc(-n2ccc3cnc(Cl)nc32)c1]

2-(3-(2-Chloro-7H-pyrrolo[2,3-d]pyrimidin-7-yl)phenyl)isothiazolidine 1,1-dioxide (AM1-125): 4-(2-bromoethyl)-1,2-dichlorobenzene (0.300 g, 0.928 mmol), 2-Chloro-7H-pyrrolo[2,3-d]pyrimidine (0.119 g, 0.774 mmol), copper iodide (0.030 g, 0.155 mmol) and potassium phosphate (0.493 g, 2.32 mmol) were dissolved with 1,4 dioxane (4 mL, 0.2 M). The flask was purged with argon and then (1R,2R)-cyclohexane-1,2-diamine (0.047 mL, 0.387 mmol) was added. The solution was stirred at 120° C. for 24 hours. The solution was diluted with ethyl acetate (50 mL), washed with ammonium chloride (2×30 mL) and brine (30 mL). The organic layer was dried over sodium sulfate and evaporated under reduced pressure. Purified by chromatography (hexane/EtOAc 70%-100% then DCM/MeOH 0%-15%) to yield a white solid (0.101 g, 37%). ¹H NMR (500 MHz, DMSO) δ 9.13 (s, 1H), 8.11 (d, J=3.8 Hz, 1H), 7.65 (t, J=8.1 Hz, 1H), 7.63-7.57 (m, 2H), 7.39 (ddd, J=8.2, 2.3, 1.0 Hz, 1H), 7.00 (d, J=3.7 Hz, 1H), 3.90 (t, J=6.5 Hz, 2H), 3.64 (t, J=7.4 Hz, 2H), 2.51 (p, 1H). HPLC-MS (ESI+) m/z, 348.9 (M+H)⁺.

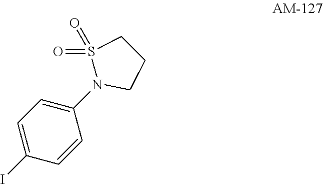 [SMILES: O=S1(=O)CCCN1c1ccc(I)cc1]

2-(4-Iodophenyl)isothiazolidine 1,1-dioxide (AM1-127): To a solution of 4-iodoaniline (1 g, 4.57 mmol) and Et₃N (1.23 mL, 8.73 mmol) in DCM (9 mL, 0.5 M), 3-chloropropane-1-sulfonyl chloride (0.75 mL, 6.15 mmol) was added. The mixture was stirred for 3 hours at 60° C. The solution was cooled to room temperature, diluted with DCM (20 mL), and washed with 1 N HCl (15 mL). The organic layer was separated from the aqueous layer, dried over sodium sulfate, and evaporated under reduced pressure. The resulting crude mixture was dissolved in DMF (7 mL, 0.6 M) and DBU (0.8 mL, 5.58 mmol) was added. After being stirred for 3 hours at room temperature, the reaction mixture was poured into 100 mL of hexane/ethyl acetate (1/1) and washed with 1 N HCl (100 mL). The organic layer was dried over sodium sulfate and evaporated under reduced pressure to yield a brown solid (1.45 g, 98%). ¹H NMR (500 MHz, DMSO) δ 7.70 (dt, 2H), 7.02 (dt, 2H), 3.72 (t, J=6.5 Hz, 2H), 3.52 (t, J=7.4 Hz, 2H), 2.40 (p, J=6.9 Hz, 2H). ¹³C NMR (126 MHz, DMSO) δ 138.61, 138.20, 120.43, 87.30, 48.77, 46.82, 18.90. HPLC-MS (ESI+) m/z, 323.8 (M+H)⁺.

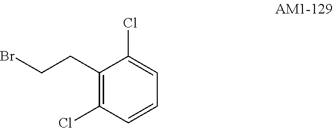 [SMILES: Clc1cccc(Cl)c1CCBr]

2-(2-Bromoethyl)-1,3-dichlorobenzene (AM1-129): 2-(2,6-Dichlorophenyl)ethan-1-ol (1 g, 5.2 mmol), NBS (1.12 g, 6.3 mmol), and PPh₃ (1.65 g, 6.3 mmol) were dissolved in DCM (25 mL, 0.21 M) and left to stir for four hours at 0° C. The solution was quenched with water (25 mL) and left to stir for ten minutes. The water was separated, and the organic layer was washed with water (25 mL) and brine (25 mL). The organic layer was dried over sodium sulfate and concentrated under reduced pressure. Purified by column chromatography of hexane to yield a clear oil (0.437 g, 33%). ¹H NMR (500 MHz, DMSO) δ 7.50 (d, J=8.1 Hz, 1H), 7.35 (ddd, J=8.4, 7.7, 1.9 Hz, 1H), 3.80 (t, J=8.0, 7.1 Hz, 1H), 3.64 (t, J=8.4, 7.1 Hz, 1H), 3.44 (t, J=8.4, 7.2 Hz, 1H), 3.36 (t, J=7.6 Hz, 1H). HPLC-MS (ESI+) m/z, 279.0 (M+H)⁺.

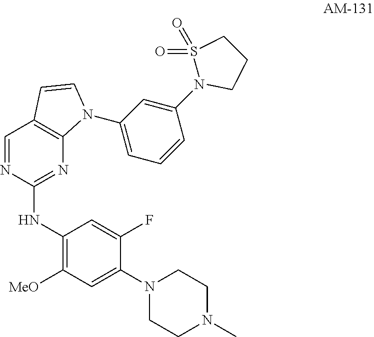 [SMILES: COc1cc(N2CCN(C)CC2)c(F)cc1Nc1ncc2ccn(-c3cccc(N4CCCS4(=O)=O)c3)c2n1]

2-(3-(2-((5-Fluoro-2-methoxy-4-(4-methylpiperazin-1-yl)phenyl)amino)-7H-pyrrolo[2,3-d]pyrimidin-7-yl)phenyl)isothiazolidine 1,1-dioxide (AM1-131): 2-(3-(2-chloro-7H-pyrrolo[2,3-d]pyrimidin-7-yl)phenyl)isothiazolidine 1,1-dioxide (0.05 g, 0.144 mmol), 5-fluoro-2-methoxy-4-(4-methylpiperazin-1-yl)aniline (0.034 g, 0.144 mmol), Pd₂(dba)₃ (0.013 g, 0.0144 mmol), Xphos (0.0137 g, 0.0288 mmol), and tert-butanol (2 mL, 0.07 M) were combined. The flask was capped and purged with argon for ten minutes. Pd₂(dba)₃ (0.013 g, 0.014 mmol) was added, and the solution was stirred at 105° C. for 24 hours. The solution was diluted with ethyl acetate (30 mL) and washed with sodium bicarbonate (20 mL×2) and then brine (30 mL). The organic layer was dried over sodium sulfate and concentrated under reduced pressure. Purified by column chromatography gradient Hexane/EtOAc 70%-100% then DCM/MeOH 0%-20% to yield the desired product as a yellow solid (0.013 g, 16%). ¹H NMR (500 MHz, DMSO) δ 8.81 (s, 1H), 8.21 (d, J=14.8 Hz, 1H), 7.86 (s, 1H), 7.67 (d, J=3.7 Hz, 1H), 7.65-7.61 (m, 1H), 7.57 (dd, J=4.6, 2.5 Hz, 1H), 7.54 (d, J=8.1 Hz, 1H), 7.29 (dd, 1H), 6.73-6.70 (m, 1H), 6.69 (s, 1H), 3.87 (s, 3H), 3.80 (t, J=6.5 Hz, 2H), 3.56 (t, J=7.4 Hz, 2H), 3.02 (s, 4H), 2.43 (p, J=6.9 Hz, 2H), 2.26 (s, 4H). ¹⁹F NMR (471 MHz, proton decoupled, DMSO) δ −131.2934. ¹⁹F NMR (471 MHz, proton coupled, DMSO) δ −131.29 (dd, J=15.0, 8.1 Hz). HPLC-MS (ESI+) m/z, 551.9 (M+H)⁺. HRMS (ESI+) m/z calculated for C₂₇H₃₀FN₇O₃S (M) 551.2115, found 551.2106.

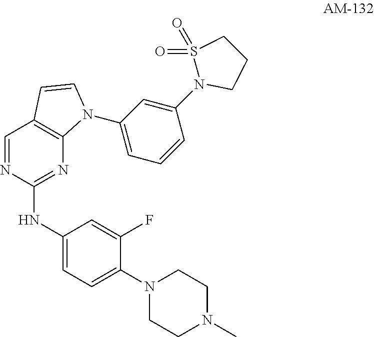 [SMILES: CN1CCN(c2ccc(Nc3ncc4ccn(-c5cccc(N6CCCS6(=O)=O)c5)c4n3)cc2F)CC1]

2-(3-(2-((3-Fluoro-4-(4-methylpiperazin-1-yl)phenyl)amino)-7H-pyrrolo[2,3-d]pyrimidin-7-yl)phenyl)isothiazolidine 1,1-dioxide (AM1-132): 2-(3-(2-chloro-7H-pyrrolo[2,3-d]pyrimidin-7-yl)phenyl)isothiazolidine 1,1-dioxide (0.04 g, 0.115 mmol), 1-(2-fluoro-4-aminophenyl)-4-methylpiperazine (0.024 g, 0.115 mmol), Xphos (0.011 g, 0.023 mmol), potassium carbonate (0.032 g, 0.23 mmol), and tert-butanol (2 mL, 0.06 M) were combined. The flask was capped and purged with argon for ten minutes. Pd₂(dba)₃ (0.011 g, 0.012 mmol) was added, and the solution was stirred at 110° C. for 24 hours. The solution was diluted with ethyl acetate (30 mL) and washed with sodium bicarbonate (20 mL×3) and then brine (30 mL). The organic layer was dried over sodium sulfate and concentrated under reduced pressure. Purified by column chromatography gradient Hexane/EtOAc 70%-100% then DCM/MeOH 0%-20% to yield the desired product as a yellow solid (0.034 g, 56%). ¹H NMR (500 MHz, DMSO) δ 9.62 (s, 1H), 8.87 (s, 1H), 7.86 (dd, J=15.7, 2.4 Hz, 1H), 7.71-7.66 (m, 2H), 7.63 (t, J=8.1 Hz, 1H), 7.58 (t, J=2.1 Hz, 1H), 7.50 (dd, J=8.7, 2.4 Hz, 1H), 7.38 (ddd, J=8.1, 2.3, 1.0 Hz, 1H), 7.00 (t, J=10.1, 8.7 Hz, 1H), 6.75 (d, J=3.7 Hz, 1H), 3.88 (t, J=6.5 Hz, 2H), 3.62 (t, J=7.4 Hz, 2H), 3.00 (s, 4H), 2.49 (p, J=6.9 Hz, 2H), 2.31 (s, 3H). ¹⁹F NMR (471 MHz, proton coupled, DMSO) δ −122.29 (dd, J=15.6, 10.2 Hz). ¹⁹F NMR (471 MHz, proton decoupled, DMSO) δ −122.29. HPLC-MS (ESI+) m/z, 521.9 (M+H)⁺. HRMS (ESI+) m/z calculated for C₂₆H₂₈FN₇O₂S (M) 521.2009, found 521.1999.

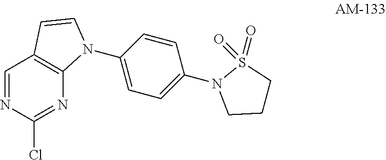 [SMILES: O=S1(=O)CCCN1c1ccc(-n2ccc3cnc(Cl)nc32)cc1]

2-(4-(2-chloro-7H-pyrrolo[2,3-d]pyrimidin-7-yl)phenyl)isothiazolidine 1,1-dioxide (AM1-133): 2-(4-iodophenyl)isothiazolidine 1,1-dioxide (0.300 g, 0.928 mmol), 2-Chloro-7H-pyrrolo[2,3-d]pyrimidine (0.119 g, 0.774 mmol), copper iodide (0.030 g, 0.155 mmol) and potassium phosphate (0.493 g, 2.32 mmol) were dissolved with 1,4 dioxane (4 mL, 0.2 M). The flask was purged with argon and then (1R,2R)-cyclohexane-1,2-diamine (0.047 mL, 0.387 mmol) was added. The solution was stirred at 120° C. for 24 hours. The solution was diluted with ethyl acetate (50 mL), washed with ammonium chloride (3×30 mL) and brine (30 mL). The organic layer was dried over sodium sulfate and evaporated under reduced pressure. The residue was purified by chromatography (hexane/EtOAc 0%-70%) to yield a white solid product (0.110 g, 41%). ¹H NMR (500 MHz, DMSO) δ 9.05 (s, 1H), 8.01 (d, J=3.7 Hz, 1H), 7.82-7.75 (m, 2H), 7.44-7.37 (m, 2H), 6.92 (d, J=3.7 Hz, 1H), 3.83 (t, J=6.5 Hz, 2H), 3.57 (t, J=7.4 Hz, 2H), 2.45 (p, J=6.9 Hz, 2H). HPLC-MS (ESI+) m/z, 348.9 (M+H)⁺.

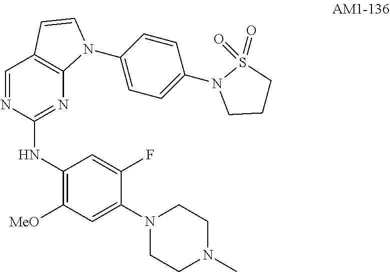 [SMILES: COc1cc(N2CCN(C)CC2)c(F)cc1Nc1ncc2ccn(-c3ccc(N4CCCS4(=O)=O)cc3)c2n1]

2-(4-(2-((5-Fluoro-2-methoxy-4-(4-methylpiperazin-1-yl)phenyl)amino)-7H-pyrrolo[2,3-d]pyrimidin-7-yl)phenyl)isothiazolidine 1,1-dioxide (AM1-136): 2-(4-(2-chloro-7H-pyrrolo[2,3-d]pyrimidin-7-yl)phenyl)isothiazolidine 1,1-dioxide (0.050 g, 0.143 mmol), 5-fluoro-2-methoxy-4-(4-methylpiperazin-1-yl)aniline (0.031 g, 0.129 mmol), Xphos (0.014 g, 0.029 mmol), potassium carbonate (0.040 g, 0.286 mmol), and tert-butanol (2 mL, 0.07 M) were combined. The flask was capped and purged with argon for ten minutes. Pd₂(dba)₃ (0.013 g, 0.014 mmol) was added, and the solution was stirred at 105° C. for 24 hours. The solution was diluted with ethyl acetate (30 mL) and washed with sodium bicarbonate (20 mL×3) and then brine (30 mL). The organic layer was dried over sodium sulfate and concentrated under reduced pressure. Purified by column chromatography gradient Hexane/EtOAc 70%-100% then DCM/MeOH 0%-5% to yield the desired product as a white solid (0.024 g, 34%). ¹H NMR (500 MHz, DMSO) δ 8.80 (s, 1H), 8.22 (d, J=15.0 Hz, 1H), 7.93-7.86 (m, 2H), 7.84 (s, 1H), 7.66 (d, J=3.7 Hz, 1H), 7.41-7.34 (m, 2H), 6.73-6.67 (m, 2H), 3.87 (s, 3H), 3.81 (t, J=6.5 Hz, 2H), 3.57 (t, J=7.4 Hz, 2H), 3.01 (s, J=4.7 Hz, 4H), 2.49-2.42 (m, 2H), 2.25 (s, 4H). ¹⁹F NMR (471 MHz, proton coupled, DMSO) δ −131.31 (dd, J=15.0, 8.1 Hz). ¹⁹F NMR (471 MHz, proton decoupled, DMSO) δ −131.31. HPLC-MS (ESI+) m/z, 552.0 (M+H)⁺. HRMS (ESI+) m/z calculated for C₂₇H₃₀FN₇O₃S (M) 551.2115, found 551.2109.

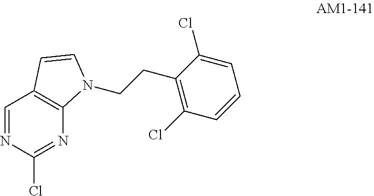 [SMILES: Clc1ncc2ccn(CCc3c(Cl)cccc3Cl)c2n1]

2-Chloro-7-(2,6-dichlorophenethyl)-7H-pyrrolo[2,3-d]pyrimidine (AM1-141): 2-Chloro-7H-pyrrolo[2,3-d]pyrimidine (0.22 g, 1.43 mmol), 2-(2,6-Dichlorophenyl)ethyl bromide (0.40 g, 1.58 mmol), and potassium carbonate (0.20 g, 1.43 mmol) were dissolved in DMF (3 mL, 0.5 M). The solution was left to stir at 100° C. for 24 hours. The solution was diluted with ethyl acetate (40 mL), washed with deionized water (20 mL×3), and washed with brine (20 mL). The organic layer was dried over sodium sulfate and concentrated under reduced pressure. The crude material was triturated with ethyl acetate/hexane (1:10), the supernatant was separated from the pellet, and the supernatant was dried under reduced pressure. The crude was purified using column chromatography gradient 0-50% ethyl acetate/hexane to yield the tan solid (0.101 g, 22%). ¹H NMR (500 MHz, DMSO) δ 8.87 (s, 1H), 7.43 (d, J=3.6 Hz, 1H), 7.39-7.35 (m, 2H), 7.26 (dd, J=8.6, 7.4 Hz, 1H), 6.61 (d, J=3.6 Hz, 1H), 4.52 (t, J=6.5 Hz, 2H), 3.38 (t, J=6.5 Hz, 2H). ¹³C NMR (126 MHz, CDCl₃) δ 152.84, 152.13, 150.22, 135.91, 133.70, 130.1802, 128.80, 128.29, 117.67, 100.17, 42.85, 31.64. HPLC-MS (ESI+) m/z, 327.9 (M+H)⁺.

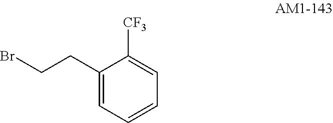 [SMILES: FC(F)(F)c1ccccc1CCBr]

1-(2-Bromoethyl)-2-(trifluoromethyl)benzene (AM1-143): 2-[2-(Trifluoromethyl)phenyl]ethanol (1 g, 5.2 mmol), NBS (1.12 g, 6.3 mmol), and PPh₃ (1.65 g, 6.3 mmol) were dissolved in DCM (20 mL, 0.3 M) and left to stir for two hours at 0° C. The solution was quenched with water (10 mL) and left to stir for ten minutes. The water was separated, and the organic layer was washed with water (15 mL×2) and brine (15 mL). The organic layer was dried over sodium sulfate and concentrated under reduced pressure. The crude residue was triturated with DCM/hexanes (1:10), separated from supernatant from the pellet, and the supernatant was dried under reduced pressure. The crude product was purified by column chromatography of hexane to yield a clear oil (0.414 g, 31%). ¹H NMR (500 MHz, DMSO) δ 7.72 (dd, J=7.8, 1.4 Hz, 1H), 7.69-7.60 (m, 2H), 7.52-7.45 (m, 1H), 3.73 (t, 2H), 3.29 (t, J=7.7 Hz, 2H). ¹⁹F NMR (471 MHz, proton decoupled and coupled, DMSO) δ −58.11. HPLC-MS (ESI+) m/z, 279.0 (M+Na)⁺.

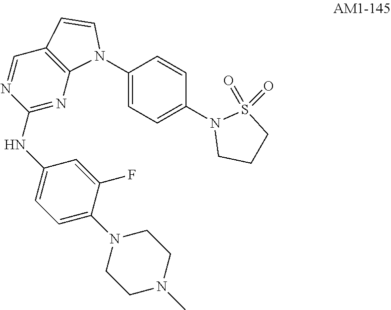 [SMILES: CN1CCN(c2ccc(Nc3ncc4ccn(-c5ccc(N6CCCS6(=O)=O)cc5)c4n3)cc2F)CC1]

2-(4-(2-((3-Fluoro-4-(4-methylpiperazin-1-yl)phenyl)amino)-7H-pyrrolo[2,3-d]pyrimidin-7-yl)phenyl)isothiazolidine 1,1-dioxide (AM1-145): 2-(4-(2-chloro-7H-pyrrolo[2,3-d]pyrimidin-7-yl)phenyl)isothiazolidine 1,1-dioxide (0.040 g, 0.115 mmol), 1-(2-Fluoro-4-aminophenyl)-4-methylpiperazine (0.024 g, 0.115 mmol), Xphos (0.011 g, 0.023 mmol), potassium carbonate (0.032 g, 0.23 mmol), and tert-butanol (2 mL, 0.06 M) were combined. The flask was capped and purged with argon for ten minutes. Pd₂(dba)₃ (0.011 g, 0.011 mmol) was added, and the solution was stirred at 110° C. for 24 hours. The solution was diluted with ethyl acetate (30 mL) and washed with sodium bicarbonate (20 mL) and then brine (20 mL). The organic layer was dried over sodium sulfate and concentrated under reduced pressure. Purified by reverse phase column chromatography gradient H₂O (0.1% formic acid)/MeOH 0%-10% to yield the desired product as a white solid (0.018 g, 30%). ¹H NMR (500 MHz, DMSO) δ 9.54 (s, 1H), 8.81 (s, 1H), 8.14 (s, 1H), 7.89-7.87 (m, 2H), 7.84 (dd, J=15.8, 2.5 Hz, 1H), 7.62 (d, J=3.7 Hz, 1H), 7.44-7.36 (m, 3H), 6.95 (t, J=9.4 Hz, 1H), 6.68 (d, J=3.7 Hz, 1H), 3.82 (t, J=6.5 Hz, 2H), 3.57 (t, J=7.4 Hz, 2H), 2.95 (t, J=4.8 Hz, 4H), 2.45 (q, J=7.0 Hz, 2H), 2.27 (s, 3H). ¹⁹F NMR (471 MHz, proton coupled, DMSO) δ −122.30 (dd, J=15.7, 9.9 Hz). ¹⁹F NMR (471 MHz, proton decoupled, DMSO) δ −122.30. HPLC-MS (ESI+) m/z, 522.0 (M+H)⁺. HRMS (ESI+) m/z calculated for C₂₆H₂₈FN₇O₂S (M) 521.2009, found 521.2007.

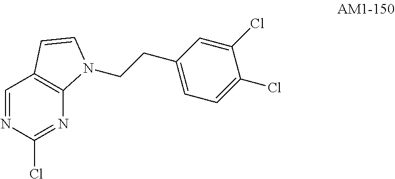 [SMILES: Clc1ncc2ccn(CCc3ccc(Cl)c(Cl)c3)c2n1]

2-Chloro-7-(3,4-dichlorophenethyl)-7H-pyrrolo[2,3-d]pyrimidine (AM1-150): 2-Chloro-7H-pyrrolo[2,3-d]pyrimidine (0.050 g, 0.328 mmol) was added to a solution of sodium hydride (0.014 g, 0.361 mmol, 60% w/w) in acetonitrile (0.3 mL) and left to stir at room temperature for 30 minutes. To the solution, 4-(2-bromoethyl)-1,2-dichlorobenzene was added and left to stir for 24 hours at room temperature. The solution was evaporated under reduced pressure, resuspended with ethyl acetate (10 mL), washed with deionized water (10 mL), and extracted with ethyl acetate (3×10 mL). The combined organic layers were dried over sodium sulfate and evaporated under reduced pressure. Purified by gradient EtOAc/hexanes 0%-35% to yield the desired product as a white solid (0.034 g, 32%). ¹H NMR (500 MHz, DMSO) δ 8.81 (s, 1H), 7.52 (d, J=3.5 Hz, 1H), 7.36 (d, J=8.2 Hz, 1H), 7.33 (d, J=2.1 Hz, 1H), 6.98 (dd, J=8.2, 2.1 Hz, 1H), 6.57 (d, J=3.6 Hz, 1H), 4.40 (t, J=6.9 Hz, 2H), 3.04 (t, J=6.9 Hz, 2H). HPLC-MS (ESI+) m/z, 327.9 (M+H)⁺.

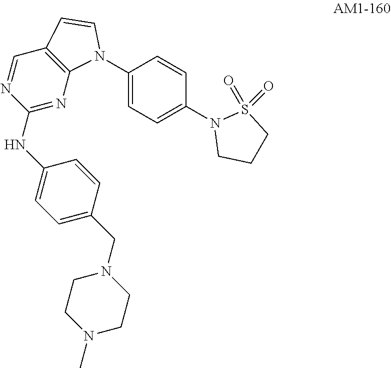 [SMILES: CN1CCN(Cc2ccc(Nc3ncc4ccn(-c5ccc(N6CCCS6(=O)=O)cc5)c4n3)cc2)CC1]

2-(4-(2-((4-((4-Methylpiperazin-1-yl)methyl)phenyl)amino)-7H-pyrrolo[2,3-d]pyrimidin-7-yl)phenyl)isothiazolidine 1,1-dioxide (AM1-160): 2-(4-(2-chloro-7H-pyrrolo[2,3-d]pyrimidin-7-yl)phenyl)isothiazolidine 1,1-dioxide (0.050 g, 0.143 mmol), 4-[(4-methyl-1-piperazinyl)methyl]aniline (0.029 g, 0.143 mmol), Xphos (0.014 g, 0.029 mmol), potassium carbonate (0.040 g, 0.286 mmol), and tert-butanol (2 mL, 0.07 M) were combined. The flask was capped and purged with argon for ten minutes. Pd₂(dba)₃ (0.013 g, 0.014 mmol) was added, and the solution was stirred at 105° C. for 18 hours. The solution was diluted with ethyl acetate (30 mL) and washed with sodium bicarbonate (20 mL) and then brine (20 mL). The organic layer was dried over sodium sulfate and concentrated under reduced pressure. Purified by reverse phase column chromatography gradient H₂O (0.1% formic acid)/MeOH 0%-30% to yield the desired product as a tan solid (0.010 g, 14%). ¹H NMR (500 MHz, DMSO) δ 9.48 (s, 1H), 8.80 (s, 1H), 7.92-7.85 (m, 2H), 7.76 (d, J=8.4 Hz, 2H), 7.62 (d, J=3.7 Hz, 1H), 7.43-7.37 (m, 2H), 7.16 (d, J=8.2 Hz, 2H), 6.67 (d, J=3.7 Hz, 1H), 3.83 (t, J=6.5 Hz, 2H), 3.57 (t, J=7.4 Hz, 2H), 2.46 (p, J=7.3 Hz, 2H), 2.21 (s, 4H). HPLC-MS (ESI+) m/z, 518.0 (M+H)⁺. HRMS (ESI+) m/z calculated for C₂₇H₃₁N₇O₂S (M) 517.226, found 517.2261.

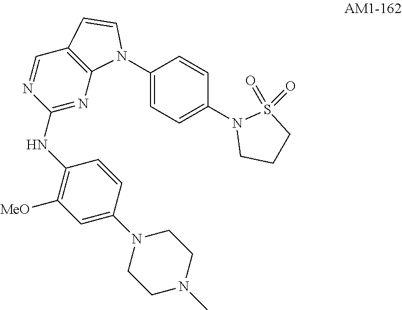 [SMILES: COc1cc(N2CCN(C)CC2)ccc1Nc1ncc2ccn(-c3ccc(N4CCCS4(=O)=O)cc3)c2n1]

2-(4-(2-((2-Methoxy-4-(4-methylpiperazin-1-yl)phenyl)amino)-7H-pyrrolo[2,3-d]pyrimidin-7-yl)phenyl)isothiazolidine 1,1-dioxide (AM1-162): 2-(4-(2-chloro-7H-pyrrolo[2,3-d]pyrimidin-7-yl)phenyl)isothiazolidine 1,1-dioxide (0.050 g, 0.143 mmol), 2-methoxy-4-(4-methylpiperazin-1-yl)aniline (0.032 g, 0.143 mmol), Xphos (0.014 g, 0.029 mmol), potassium carbonate (0.040 g, 0.286 mmol), and tert-butanol (2 mL, 0.07 M) were combined. The flask was capped and purged with argon for ten minutes. Pd₂(dba)₃ (0.013 g, 0.014 mmol) was added, and the solution was stirred at 105° C. for 18 hours. The solution was diluted with ethyl acetate (30 mL), washed with sodium bicarbonate (20 mL) and then brine (20 mL). The organic layer was dried over sodium sulfate and concentrated under reduced pressure. Purified column chromatography gradient DCM/MeOH 0%-5% to yield the desired product as a tan solid (0.016 g, 21%). HPLC-MS (ESI+) m/z, 534.0 (M+H)⁺. HRMS (ESI+) m/z calculated for C₂₇H₃₁N₇O₃S (M) 533.2209, found 533.2206.

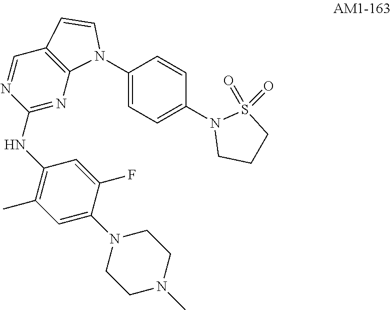 [SMILES: Cc1cc(N2CCN(C)CC2)c(F)cc1Nc1ncc2ccn(-c3ccc(N4CCCS4(=O)=O)cc3)c2n1]

2-(4-(2-((5-Fluoro-2-methyl-4-(4-methylpiperazin-1-yl)phenyl)amino)-7H-pyrrolo[2,3-d]pyrimidin-7-yl)phenyl)isothiazolidine 1,1-dioxide (AM1-163): 2-(4-(2-chloro-7H-pyrrolo[2,3-d]pyrimidin-7-yl)phenyl)isothiazolidine 1,1-dioxide (0.050 g, 0.143 mmol), 5-fluoro-2-methyl-4-(4-methylpiperazin-1-yl)aniline (0.032 g, 0.143 mmol), Xphos (0.014 g, 0.029 mmol), potassium carbonate (0.040 g, 0.286 mmol), and tert-butanol (2 mL, 0.07 M) were combined. The flask was capped and purged with argon for ten minutes. Pd₂(dba)₃ (0.013 g, 0.014 mmol) was added, and the solution was stirred at 105° C. for 18 hours. The solution was diluted with ethyl acetate (30 mL), washed with sodium bicarbonate (20 mL) and then brine (20 mL). The organic layer was dried over sodium sulfate and concentrated under reduced pressure. Purified by column chromatography gradient EtOAc/hexanes 70-100% then DCM/MeOH 0%-5% to yield the desired product as a tan solid (0.016 g, 21%). ¹H NMR (600 MHz, DMSO) δ 8.72 (s, 1H), 8.46 (s, 1H), 7.88-7.83 (m, 2H), 7.60 (d, J=3.7 Hz, 1H), 7.55 (d, J=14.8 Hz, 1H), 7.32-7.27 (m, 2H), 6.84 (d, J=9.9 Hz, 1H), 6.64 (d, J=3.7 Hz, 1H), 3.78 (t, J=6.5 Hz, 2H), 3.55 (t, J=7.4 Hz, 2H), 2.98 (s, 4H), 2.4413 (p, J=6.9 Hz, 2H), 2.24 (s, 4H), 2.19 (s, 3H). ¹⁹F NMR (471 MHz, proton decoupled, DMSO) δ −126.59. ¹⁹F NMR (471 MHz, proton coupled, DMSO) δ −126.59 (dd, J=14.9, 9.7 Hz). HPLC-MS (ESI+) m/z, 536.0 (M+H)⁺. HRMS (ESI+) m/z calculated for C₂₇H₃₀N₇O₂S (M) 535.2166, found 535.2162.

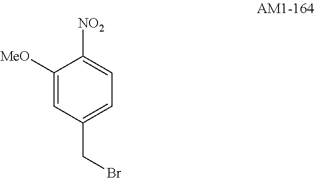 [SMILES: COc1cc(CBr)ccc1[N+](=O)[O-]]

4-Bromomethyl-2-methoxy-1-nitrobenzene (AM1-164-B2): N-Bromosuccinimide (2.56 g, 14.36 mmol) was added in portions over a period of 5 minutes to a well stirred solution of 4-methyl-2methoxy-1-nitro benzene (2.0 g, 11.96 mmol) and 1,1′-azobis(cyclohexanecarbonitrile) (0.039 g, 0.239 mmol) in dichloroethane (20 mL, 0.6 M) and the resulting mixture was heated to reflux under UV light for 3 hours. After cooling to room temperature, the solution was diluted with 60 mL of DCM, washed with sodium bicarbonate (40 mL), water (40 mL), and brine (40 mL). The organic layer was dried over sodium sulfate and evaporated under reduced pressure. The residue was used in the next step without further purification. ¹H NMR (600 MHz, DMSO) δ 7.88 (d, J=8.3 Hz, 1H), 7.48 (d, J=1.7 Hz, 1H), 7.21-7.18 (m, 1H), 4.74 (s, 2H), 3.93 (s, 3H). HPLC-MS (ESI+) m/z, 269.9 (M+Na)⁺.

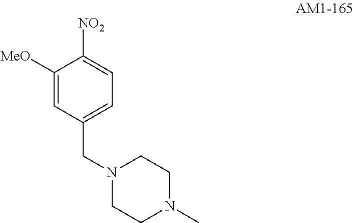 [SMILES: COc1cc(CN2CCN(C)CC2)ccc1[N+](=O)[O-]]

1-(3-Methoxy-4-nitrobenzyl)-4-methylpiperazine (AM1-165): 4-Bromomethyl-2-methoxy-1-nitrobenzene (2.75 g, 11.176 mmol), Et₃N (3.115 mL, 22.35 mmol), and 1-Methylpiperazine (2.48 mL, 22.35 mmol) were dissolved in dry DCM (20 mL, 0.56 M) stirred at room temperature for 1 hour. The solution was diluted with DCM (20 mL), washed with water (3×40 mL), and 1 N HCl (40 mL). The acidic aqueous layer was re-extracted with DCM (3×40 mL) and the organic layers were combined, basified with sodium bicarbonate (40 mL), dried over sodium sulfate, and concentrated under reduced pressure to yield the desired product as a dark orange oil (1.6 g, 54%). ¹H NMR (600 MHz, DMSO) δ 7.84 (d, J=8.2 Hz, 1H), 7.26 (d, J=1.6 Hz, 1H), 7.05 (dd, J=8.3, 1.5 Hz, 1H), 3.91 (s, 3H), 3.54 (s, 2H), 2.43-2.25 (m, 4H), 2.15 (s, 3H). HPLC-MS (ESI+) m/z, 266.1 (M+H)⁺.

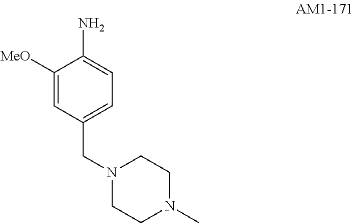 [SMILES: COc1cc(CN2CCN(C)CC2)ccc1N]

2-Methoxy-4-((4-methylpiperazin-1-yl)methyl)aniline (AM1-171): Palladium on carbon (0.377 g, 10 wt %) was added to a flask and purged with argon. Methanol (20 mL) was added and the solution was degassed for 10 minutes. 1-(3-methoxy-4-nitrobenzyl)-4-methylpiperazine (1 g, 3.77 mmol) was dissolved in methanol (10 mL) and added to the flask. The solution was evacuated and filled with argon (4×). A hydrogen balloon was attached to the reaction vessel. The vessel was evacuated and filled with hydrogen (4×). The hydrogen was bubbled through the solution, the flask was evacuated once more, and the solution was stirred at 50 C for 24 hours. Palladium on carbon (0.100 g, 10 wt %) was added to a flask, and the solution was stirred for an additional 24 hours. The solution was filtered through celite and concentrated under reduced pressure. Purified by gradient column chromatography 0-20% MeOH/DCM to yield the desired product as a dark brown solid (0.531 g, 60%). ¹H NMR (500 MHz, DMSO) δ 6.70 (d, J=1.6 Hz, 1H), 6.61-6.52 (m, 2H), 4.59 (s, 2H), 3.74 (s, 3H), 3.29 (s, 2H), 2.33 (s, 4H), 2.16 (s, 3H). HPLC-MS (ESI+) m/z, 236.2 (M+H)⁺.

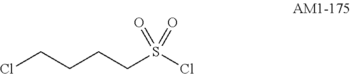

4-Chlorobutane-1-sulfonyl chloride (AM1-175): 1,2-oxathiane 2,2-dioxide (2.5 g, 0.018 mol) was added to a round bottom flask and purged with argon. SOCl₂ (4.5 mL, 3.3 eq) and DMF (0.5 mL) were added and stirred under argon at 80° C. for 20 hours. The reaction was cooled to room temperature, diluted with DCM, and evaporated under reduced pressure with a sodium hydroxide base trap. This process of dilution and evaporation was repeated twice to yield a dark yellow oil (3.25 g, 92%). ¹H NMR (500 MHz, CDCl3) δ 3.78-3.71 (m, 2H), 3.63 (t, J=6.2 Hz, 2H), 2.32-2.22 (m, 2H), 2.07-2.00 (m, 2H).

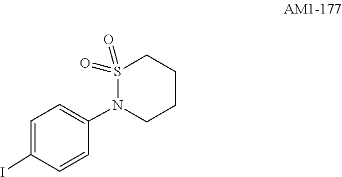 [SMILES: O=S1(=O)CCCCN1c1ccc(I)cc1]

2-(4-Iodophenyl)-1,2-thiazinane 1,1-dioxide (AM1-177): To a solution of 4-iodoaniline (1.5 g, 6.848 mmol) and Et₃N (1.81 mL, 1.32 mmol) in DCM (13 mL), 4-chlorobutane-1-sulfonyl chloride (1.7 g, 8.903 mmol) was added. The mixture was stirred for 24 hours at 50° C., cooled to room temperature, diluted with DCM (20 mL), washed with 1 N HCl (20 mL), and evaporated under reduced pressure. The resulting crude mixture was dissolved in DMF (10 mL) and DBU (1.22 mL, 8.218 mmol) was added. After being stirred for 3 hours at room temperature, the reaction mixture was poured into 30 mL of hexane/ethyl acetate (1/1), washed with 1 N HCl (20 mL), dried over sodium sulfate, and evaporated under reduced pressure to yield a brown solid (1.777 g, 77%). ¹H NMR (500 MHz, DMSO) δ 7.73 (d, J=8.5 Hz, 2H), 7.12 (d, J=8.5 Hz, 2H), 3.65-3.61 (m, 2H), 3.31-3.26 (m, 2H), 2.15 (ddt, J=12.1, 8.5, 4.9 Hz, 2H), 1.80 (ddt, J=11.7, 8.3, 4.6 Hz, 2H). ¹³C NMR (126 MHz, DMSO) δ 140.94, 138.13, 129.23, 92.64, 53.33, 50.42, 24.32, 23.87. HPLC-MS (ESI+) m/z, 337.8 (M+H)⁺.

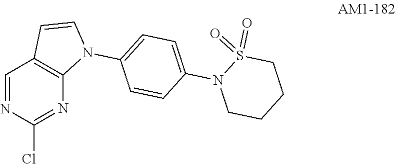 [SMILES: O=S1(=O)CCCCN1c1ccc(-n2ccc3cnc(Cl)nc32)cc1]

2-(4-(2-Chloro-7H-pyrrolo[2,3-d]pyrimidin-7-yl)phenyl)-1,2-thiazinane 1,1-dioxide (AM1-182): 2-(4-iodophenyl)-1,2-thiazinane 1,1-dioxide (0.092 g, 0.297 mmol), 2-chloro-7H-pyrrolo[2,3-d]pyrimidine (0.038 g, 0.247 mmol), copper iodide (0.0094 g, 0.049 mmol) and potassium phosphate (0.157 g, 0.741 mmol) were dissolved with 1,4 dioxane (1 mL, 0.3 M). The flask was purged with argon and then (1R,2R)-cyclohexane-1,2-diamine (0.0089 mL, 0.074 mmol) was added. The solution was stirred at 100° C. for 24 hours. The solution was diluted with ethyl acetate (20 mL), washed with ammonium chloride (15 mL) and brine (15 mL). The organic layer was dried over sodium sulfate and evaporated under reduced pressure. A second batch was prepared: 2-(4-iodophenyl)-1,2-thiazinane 1,1-dioxide (0.500 g, 1.48 mmol), 2-Chloro-7H-pyrrolo[2,3-d]pyrimidine (0.228 g, 1.48 mmol), copper iodide (0.051 g, 0.44 mmol) and potassium phosphate (0.057 g, 0.297 mmol) were dissolved with 1,4 dioxane (5 mL, 0.3 M). The flask was purged with argon and then (1R,2R)-cyclohexane-1,2-diamine (0.0534 mL, 0.44 mmol) was added. The solution was stirred at 100° C. for 24 hours. The solution was diluted with ethyl acetate (50 mL), washed with ammonium chloride (25 mL) and brine (25 mL). The organic layer was dried over sodium sulfate and evaporated under reduced pressure. The residues from the two reactions were combined and purified by column chromatography gradient DCM/MeOH 0-20% to yield a white solid product (0.311 g, 50%). ¹H NMR (500 MHz, DMSO) δ 9.06 (s, 1H), 8.05 (d, J=3.7 Hz, 1H), 7.84-7.77 (m, 2H), 7.57-7.50 (m, 2H), 6.94 (d, J=3.7 Hz, 1H), 3.77-3.71 (m, 2H), 2.23-2.15 (m, 2H), 1.89-1.83 (m, 3H); HPLC-MS (ESI+) m/z, 363.2 (M+H)+.

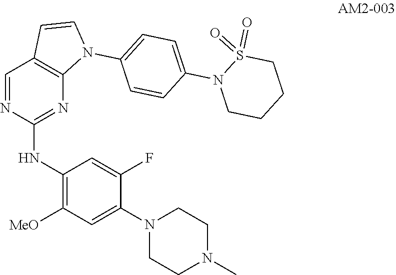 [SMILES: COc1cc(N2CCN(C)CC2)c(F)cc1Nc1ncc2ccn(-c3ccc(N4CCCCS4(=O)=O)cc3)c2n1]

2-(4-(2-((5-Fluoro-2-methoxy-4-(4-methylpiperazin-1-yl)phenyl)amino)-7H-pyrrolo[2,3-d]pyrimidin-7-yl)phenyl)-1,2-thiazinane 1,1-dioxide (AM2-003): 2-(4-(2-chloro-7H-pyrrolo[2,3-d]pyrimidin-7-yl)phenyl)-1,2-thiazinane 1,1-dioxide (0.050 g, 0.138 mmol), 5-fluoro-2-methoxy-4-(4-methylpiperazin-1-yl)aniline (0.036 g, 0.152 mmol), Xphos (0.013 g, 0.028 mmol), potassium carbonate (0.038 g, 0.276 mmol), and tert-butanol (1.5 mL, 0.09 M) were combined. The flask was capped and purged with argon for ten minutes. Pd₂(dba)₃ (0.013 g, 0.014 mmol) was added, and the solution was stirred at 100° C. for 24 hours. The solution was diluted with ethyl acetate (25 mL) and washed with sodium bicarbonate (15 mL×3) and then brine (15 mL). The organic layer was dried over sodium sulfate and concentrated under reduced pressure. Purified by column chromatography gradient DCM/MeOH 0%-5% to yield a tan solid (0.033 g, 42%). ¹H NMR (500 MHz, DMSO) δ 8.81 (s, 1H), 8.20 (d, J=15.0 Hz, 1H), 7.93-7.90 (m, 2H), 7.87 (s, 1H), 7.69 (d, J=3.7 Hz, 1H), 7.52-7.45 (m, 2H), 6.71 (d, J=3.8 Hz, 2H), 3.87 (s, 3H), 3.74-3.68 (m, 3H), 3.00 (t, J=4.8 Hz, 4H), 2.23 (s, 3H), 2.21-2.16 (m, 2H), 1.86 (ddt, J=8.2, 5.7, 3.0 Hz, 2H). ¹⁹F NMR (471 MHz, proton decoupled, DMSO) δ −131.52. ¹⁹F NMR (471 MHz, proton coupled, DMSO) δ −131.52 (dd, J=15.1, 8.2 Hz). HPLC-MS (ESI+) m/z, 566.2 (M+H)⁺. HRMS (ESI+) m/z calculated for C₂₈H₃₂N₇O₃S (M) 565.2271, found 565.2273.

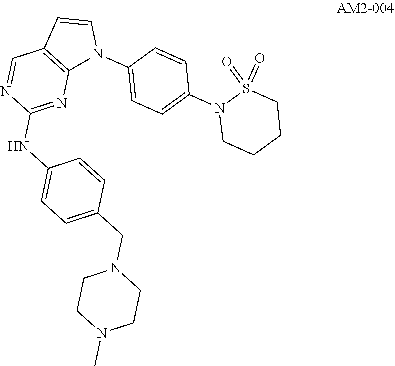 [SMILES: CN1CCN(Cc2ccc(Nc3ncc4ccn(-c5ccc(N6CCCCS6(=O)=O)cc5)c4n3)cc2)CC1]

2-(4-(2-((4-((4-Methylpiperazin-1-yl)methyl)phenyl)amino)-7H-pyrrolo[2,3-d]pyrimidin-7-yl)phenyl)-1,2-thiazinane 1,1-dioxide (AM2-004): 2-(4-(2-chloro-7H-pyrrolo[2,3-d]pyrimidin-7-yl)phenyl)-1,2-thiazinane 1,1-dioxide (0.050 g, 0.138 mmol), 4-((4-methylpiperazin-1-yl)methyl)aniline (0.031 g, 0.152 mmol), Xphos (0.013 g, 0.028 mmol), potassium carbonate (0.038 g, 0.276 mmol), and tert-butanol (1.5 mL, 0.09 M) were combined. The flask was capped and purged with argon for ten minutes. Pd₂(dba)₃ (0.013 g, 0.014 mmol) was added, and the solution was stirred at 100° C. for 18 hours. 1,4-dioxane (1 mL) was added to the reaction, and the solution stirred at 100° C. for 18 hours. Additional 1,4-dioxane (0.5 mL) and Pd₂(dba)₃ (10 mol %) was added, and the solution stirred at 120° C. for 18 hours. The solution was diluted with ethyl acetate (25 mL) and washed with sodium bicarbonate (15 mL×3) and then brine (15 mL). The organic layer was dried over sodium sulfate and concentrated under reduced pressure. Purified by column chromatography gradient DCM/MeOH 0%-20% to yield a yellow solid (0.025 g, 34%). ¹H NMR (500 MHz, DMSO) δ 9.49 (s, 1H), 8.81 (s, 1H), 7.95-7.88 (m, 2H), 7.75 (d, J=8.2 Hz, 2H), 7.66 (d, J=3.7 Hz, 1H), 7.54-7.47 (m, 2H), 7.18 (d, J=8.2 Hz, 2H), 6.69 (d, J=3.7 Hz, 1H), 3.72 (t, J=5.6 Hz, 2H), 2.25 (s, 3H), 2.23-2.16 (m, 2H), 1.87 (t, J=4.6 Hz, 2H). HPLC-MS (ESI+) m/z, 532.3 (M+H)⁺. HRMS (ESI+) m/z calculated for C₂₈H₃₃N₇O₂S (M) 531.2416, found 531.2417.

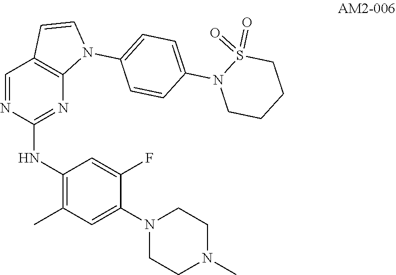 [SMILES: Cc1cc(N2CCN(C)CC2)c(F)cc1Nc1ncc2ccn(-c3ccc(N4CCCCS4(=O)=O)cc3)c2n1]

2-(4-(2-((5-Fluoro-2-methyl-4-(4-methylpiperazin-1-yl)phenyl)amino)-7H-pyrrolo[2,3-d]pyrimidin-7-yl)phenyl)-1,2-thiazinane 1,1-dioxide (AM2-006): 2-(4-(2-chloro-7H-pyrrolo[2,3-d]pyrimidin-7-yl)phenyl)-1,2-thiazinane 1,1-dioxide (0.050 g, 0.138 mmol), 5-fluoro-2-methyl-4-(4-methylpiperazin-1-yl)aniline (0.034 g, 0.152 mmol), Xphos (0.013 g, 0.028 mmol), potassium carbonate (0.038 g, 0.276 mmol), and tert-butanol (1.5 mL, 0.09 M) were combined. The flask was capped and purged with argon for ten minutes. Pd₂(dba)₃ (0.013 g, 0.014 mmol) was added, and the solution was stirred at 110° C. for 24 hours. The solution was diluted with ethyl acetate (25 mL) and washed with sodium bicarbonate (15 mL×3) and then brine (15 mL). The organic layer was dried over sodium sulfate and concentrated under reduced pressure. Purified by column chromatography gradient EtOAc/Hexane 80-100% then DCM/MeOH 0%-5% to yield a tan solid (0.013 g, 27%). ¹H NMR (500 MHz, DMSO) δ 8.73 (s, 1H), 8.50 (s, 1H), 7.89 (d, J=8.4 Hz, 2H), 7.63 (d, J=3.8 Hz, 1H), 7.54 (d, J=14.8 Hz, 1H), 7.42 (d, J=8.6 Hz, 3H), 6.85 (d, J=9.9 Hz, 1H), 6.66 (d, J=3.7 Hz, 1H), 3.68 (t, J=5.5 Hz, 2H), 2.98 (t, J=4.9 Hz, 4H), 2.23 (s, 4H), 2.19 (d, J=8.9 Hz, 6H), 1.85 (p, J=6.1 Hz, 2H).). ¹⁹F NMR (471 MHz, proton decoupled, DMSO) δ −126.71. ¹⁹F NMR (471 MHz, proton coupled, DMSO) δ −126.71 (dd, J=14.9, 9.9 Hz). HPLC-MS (ESI+) m/z, 550.3 (M+H)⁺. HRMS (ESI+) m/z calculated for C₂₈H₃₂N₇O₃S (M) 549.2322, found 549.232.

The following biological activity data was obtained for the below representative compounds of the present disclosure:

| | | DSF | |
|---|---|---|---|
| | ACK1 IC₅₀ | (ΔTm | Cell viability |
| Compound | (nM) | (° C.) | EC50 |
| 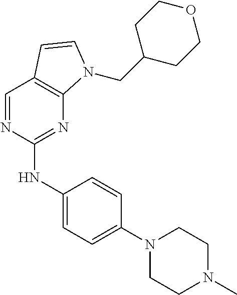 [SMILES: CN1CCN(c2ccc(Nc3ncc4ccn(CC5CCOCC5)c4n3)cc2)CC1] | <250 nM nM | | |
| 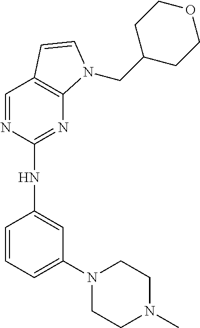 [SMILES: CN1CCN(c2cccc(Nc3ncc4ccn(CC5CCOCC5)c4n3)c2)CC1] | <500 nM nM | | |
| 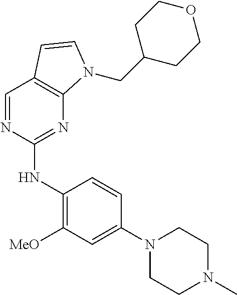 [SMILES: COc1cc(N2CCN(C)CC2)ccc1Nc1ncc2ccn(CC3CCOCC3)c2n1] | <250 nM | | |
| 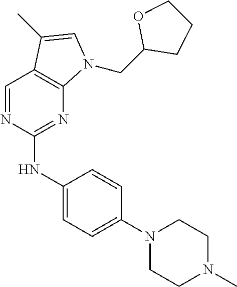 [SMILES: Cc1cn(CC2CCCO2)c2nc(Nc3ccc(N4CCN(C)CC4)cc3)ncc12] | <250 nM | | |
| 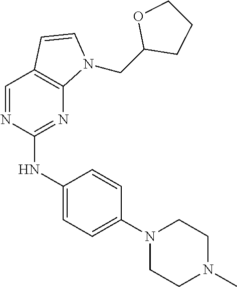 [SMILES: CN1CCN(c2ccc(Nc3ncc4ccn(CC5CCCO5)c4n3)cc2)CC1] | <250 nM | | |
| 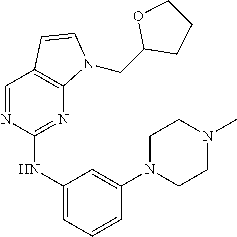 [SMILES: CN1CCN(c2cccc(Nc3ncc4ccn(CC5CCCO5)c4n3)c2)CC1] | <250 nM | | |
| 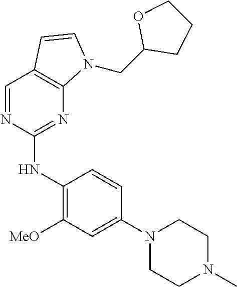 [SMILES: COc1cc(N2CCN(C)CC2)ccc1Nc1ncc2ccn(CC3CCCO3)c2n1] | <250 nM | | |
| 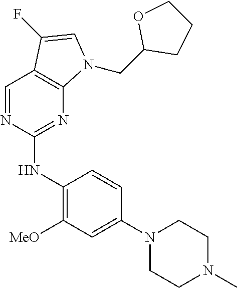 [SMILES: COc1cc(N2CCN(C)CC2)ccc1Nc1ncc2c(F)cn(CC3CCCO3)c2n1] | <250 nM | | |
| 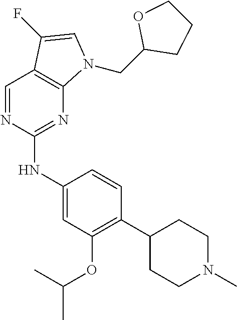 [SMILES: CC(C)Oc1cc(Nc2ncc3c(F)cn(CC4CCCO4)c3n2)ccc1C1CCN(C)CC1] | <250 nM | | |
| 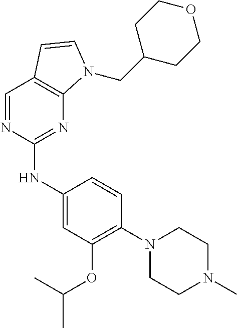 [SMILES: CC(C)Oc1cc(Nc2ncc3ccn(CC4CCOCC4)c3n2)ccc1N1CCN(C)CC1] | <250 nM | | 3.1 uM (UKE1) 3.5 uM (MDA-MB- 231) |
| 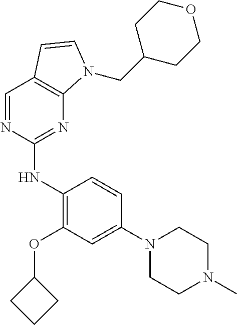 [SMILES: CN1CCN(c2ccc(Nc3ncc4ccn(CC5CCOCC5)c4n3)c(OC3CCC3)c2)CC1] | <250 nM | | |
| 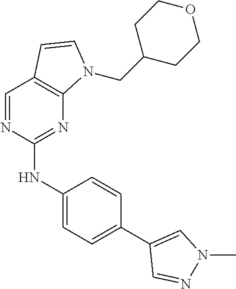 [SMILES: Cn1cc(-c2ccc(Nc3ncc4ccn(CC5CCOCC5)c4n3)cc2)cn1] | <250 nM | | |
| 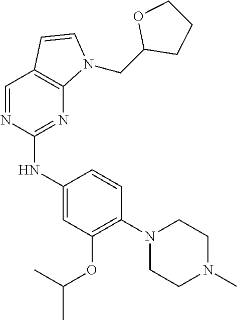 [SMILES: CC(C)Oc1cc(Nc2ncc3ccn(CC4CCCO4)c3n2)ccc1N1CCN(C)CC1] | <250 nM | | 1.1 uM (UKE1) 2.0 uM (MDA-MB- 231) |
| 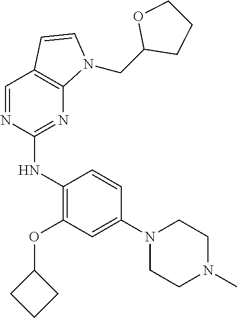 [SMILES: CN1CCN(c2ccc(Nc3ncc4ccn(CC5CCCO5)c4n3)c(OC3CCC3)c2)CC1] | <250 nM | | |
| 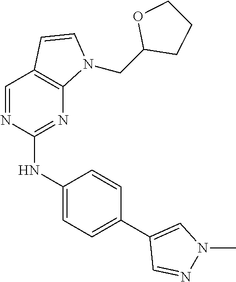 [SMILES: Cn1cc(-c2ccc(Nc3ncc4ccn(CC5CCCO5)c4n3)cc2)cn1] | <250 nM | | |
| 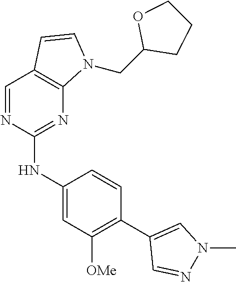 [SMILES: COc1cc(Nc2ncc3ccn(CC4CCCO4)c3n2)ccc1-c1cnn(C)c1] | <250 nM | | |
| 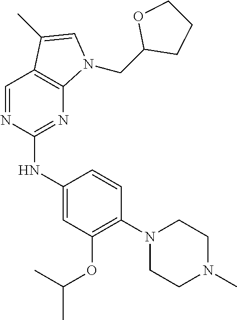 [SMILES: Cc1cn(CC2CCCO2)c2nc(Nc3ccc(N4CCN(C)CC4)c(OC(C)C)c3)ncc12] | <250 nM | | |
| 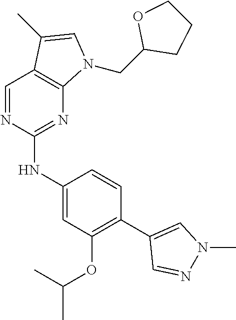 [SMILES: Cc1cn(CC2CCCO2)c2nc(Nc3ccc(-c4cnn(C)c4)c(OC(C)C)c3)ncc12] | <500 nM | 2.1 | |
| 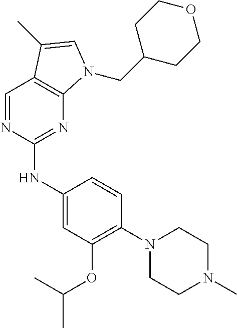 [SMILES: Cc1cn(CC2CCOCC2)c2nc(Nc3ccc(N4CCN(C)CC4)c(OC(C)C)c3)ncc12] | <250 nM | | |
| 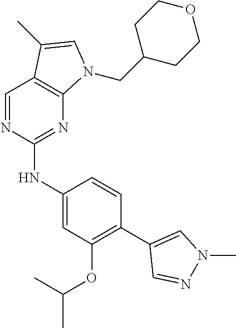 [SMILES: Cc1cn(CC2CCOCC2)c2nc(Nc3ccc(-c4cnn(C)c4)c(OC(C)C)c3)ncc12] | <250 nM | | |
| 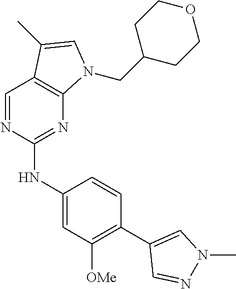 [SMILES: COc1cc(Nc2ncc3c(C)cn(CC4CCOCC4)c3n2)ccc1-c1cnn(C)c1] | <250 nM | | |
| 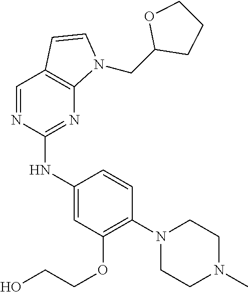 [SMILES: CN1CCN(c2ccc(Nc3ncc4ccn(CC5CCCO5)c4n3)cc2OCCO)CC1] | <250 nM | 5.3 | 1.28 uM (UKE1) 0.71 uM (UKE1) 0.72 uM (MDA-MB- 231) 1.28 uM (MDA-MB- 231) |
| 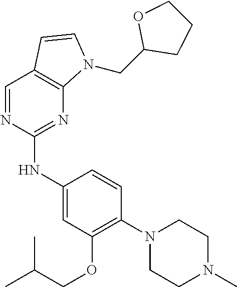 [SMILES: CC(C)COc1cc(Nc2ncc3ccn(CC4CCCO4)c3n2)ccc1N1CCN(C)CC1] | <250 nM | | |
| 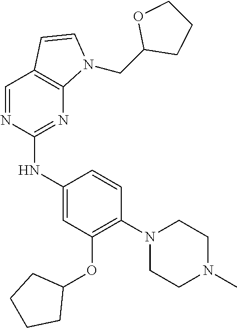 [SMILES: CN1CCN(c2ccc(Nc3ncc4ccn(CC5CCCO5)c4n3)cc2OC2CCCC2)CC1] | <250 nM | | 2.1 uM (UKE1) 2.10 uM (MDA-MB- 231) 1.05 uM (MDA-MB- 231) |
| 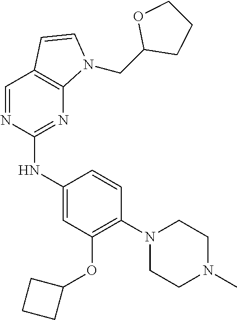 [SMILES: CN1CCN(c2ccc(Nc3ncc4ccn(CC5CCCO5)c4n3)cc2OC2CCC2)CC1] | <250 nM | | 1.72 uM (UKE1) 1.5 uM (UKE1) 1.50 uM (MDA-MB- 231) 1.71 uM (MDA-MB- 231) |
| 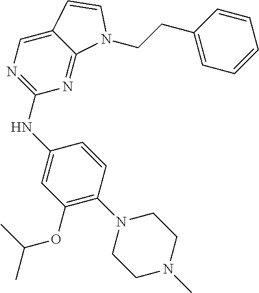 [SMILES: CC(C)Oc1cc(Nc2ncc3ccn(CCc4ccccc4)c3n2)ccc1N1CCN(C)CC1] | <250 nM | 4.5 | 1.8 uM (UKE1) 3.42 uM (MDA-MB- 231) |
| 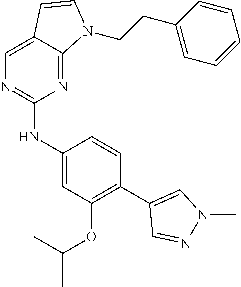 [SMILES: CC(C)Oc1cc(Nc2ncc3ccn(CCc4ccccc4)c3n2)ccc1-c1cnn(C)c1] | <750 nM | | |
| 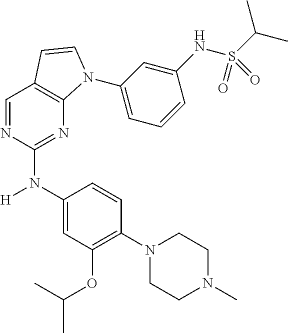 [SMILES: CC(C)Oc1cc(Nc2ncc3ccn(-c4cccc(NS(=O)(=O)C(C)C)c4)c3n2)ccc1N1CCN(C)CC1] | <250 nM | 5.2 | 0.31 uM (UKE1) 164 nM (MDA-MB- 231) |
| 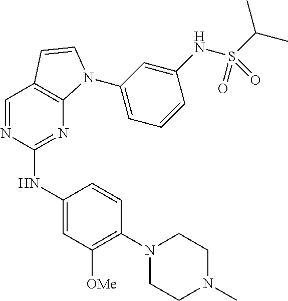 [SMILES: COc1cc(Nc2ncc3ccn(-c4cccc(NS(=O)(=O)C(C)C)c4)c3n2)ccc1N1CCN(C)CC1] | <250 nM | 6.4 | 0.11 uM (UKE1) 231 nM (MDA-MB- 231) |
| 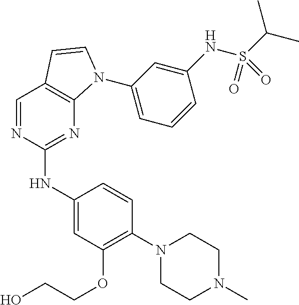 [SMILES: CC(C)S(=O)(=O)Nc1cccc(-n2ccc3cnc(Nc4ccc(N5CCN(C)CC5)c(OCCO)c4)nc32)c1] | <250 nM | 6.4 | 0.39 uM (UKE1) 151 nM (MDA-MB- 231) |
| 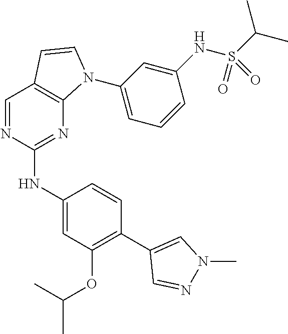 [SMILES: CC(C)Oc1cc(Nc2ncc3ccn(-c4cccc(NS(=O)(=O)C(C)C)c4)c3n2)ccc1-c1cnn(C)c1] | <250 nM | | |
| 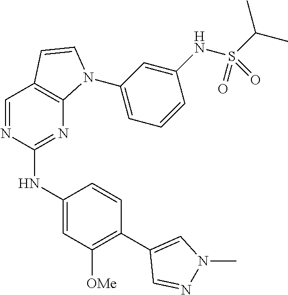 [SMILES: COc1cc(Nc2ncc3ccn(-c4cccc(NS(=O)(=O)C(C)C)c4)c3n2)ccc1-c1cnn(C)c1] | <250 nM | | |
| 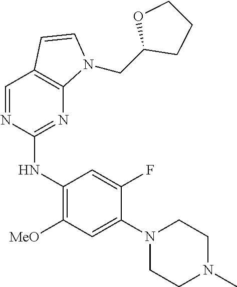 [SMILES: COc1cc(N2CCN(C)CC2)c(F)cc1Nc1ncc2ccn(C[C@H]3CCCO3)c2n1] | <250 nM | | |
| 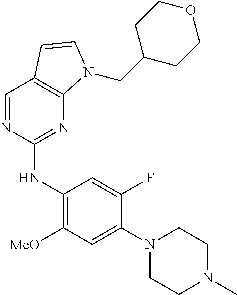 [SMILES: COc1cc(N2CCN(C)CC2)c(F)cc1Nc1ncc2ccn(CC3CCOCC3)c2n1] | <250 nM | | >10 uM (UKE1) >10 uM (MDA-MB- 231) |
| 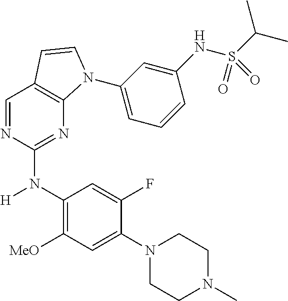 [SMILES: COc1cc(N2CCN(C)CC2)c(F)cc1Nc1ncc2ccn(-c3cccc(NS(=O)(=O)C(C)C)c3)c2n1] | <250 nM | 4.8 | 0.94 uM (UKE1) 1.58 uM (MDA-MB- 231) |
| 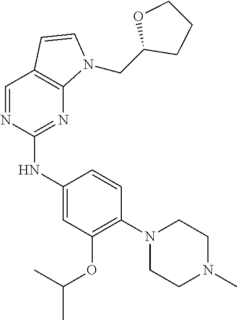 [SMILES: CC(C)Oc1cc(Nc2ncc3ccn(C[C@H]4CCCO4)c3n2)ccc1N1CCN(C)CC1] | <250 nM | 5.3 | 1.0 uM (UKE1) 1.1 uM (MDA-MB- 231) |
| 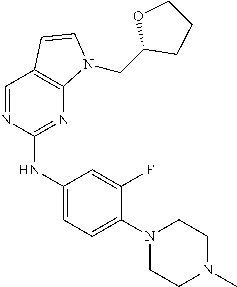 [SMILES: CN1CCN(c2ccc(Nc3ncc4ccn(C[C@H]5CCCO5)c4n3)cc2F)CC1] | <250 nM | | |
| 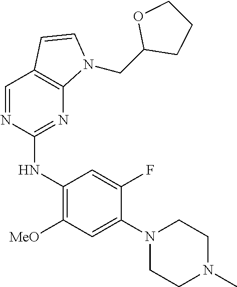 [SMILES: COc1cc(N2CCN(C)CC2)c(F)cc1Nc1ncc2ccn(CC3CCCO3)c2n1] | <250 nM | | |
| 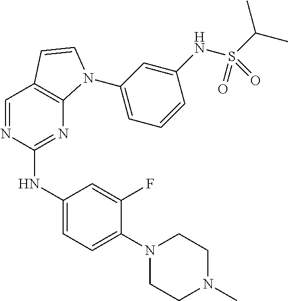 [SMILES: CC(C)S(=O)(=O)Nc1cccc(-n2ccc3cnc(Nc4ccc(N5CCN(C)CC5)c(F)c4)nc32)c1] | <250 nM | 4.5 | 0.074 uM (UKE1) 462 nM (MDA-MB- 231) |
| 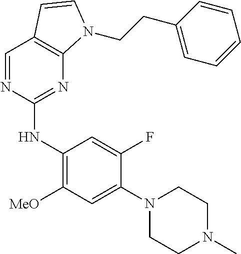 [SMILES: COc1cc(N2CCN(C)CC2)c(F)cc1Nc1ncc2ccn(CCc3ccccc3)c2n1] | <250 nM | | |
| 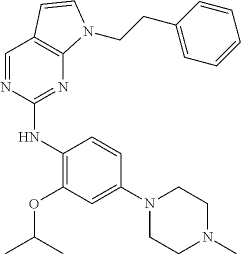 [SMILES: CC(C)Oc1cc(N2CCN(C)CC2)ccc1Nc1ncc2ccn(CCc3ccccc3)c2n1] | <1000 nM | | |
| 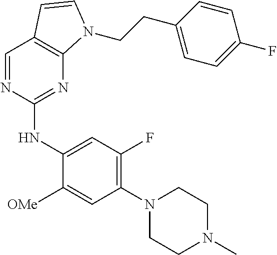 [SMILES: COc1cc(N2CCN(C)CC2)c(F)cc1Nc1ncc2ccn(CCc3ccc(F)cc3)c2n1] | <250 nM | | |
| 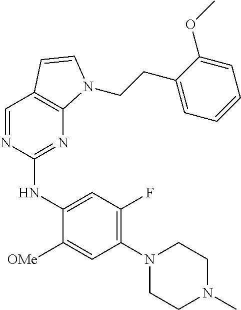 [SMILES: COc1ccccc1CCn1ccc2cnc(Nc3cc(F)c(N4CCN(C)CC4)cc3OC)nc21] | <250 nM | | |
| 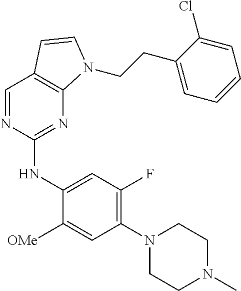 [SMILES: COc1cc(N2CCN(C)CC2)c(F)cc1Nc1ncc2ccn(CCc3ccccc3Cl)c2n1] | <250 nM | | |
| 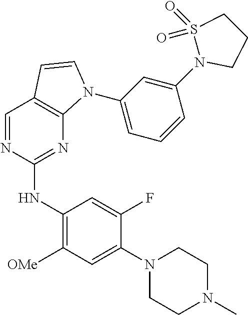 [SMILES: COc1cc(N2CCN(C)CC2)c(F)cc1Nc1ncc2ccn(-c3cccc(N4CCCS4(=O)=O)c3)c2n1] | <250 nM | | |
| 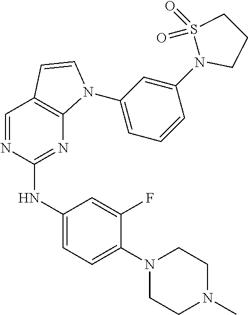 [SMILES: CN1CCN(c2ccc(Nc3ncc4ccn(-c5cccc(N6CCCS6(=O)=O)c5)c4n3)cc2F)CC1] | <250 nM | | 0.055 μM (UKE1) 0.84 uM (MDA-MB- 231) |
| 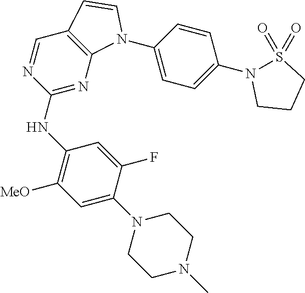 [SMILES: COc1cc(N2CCN(C)CC2)c(F)cc1Nc1ncc2ccn(-c3ccc(N4CCCS4(=O)=O)cc3)c2n1] | <250 nM | 5.2 | 1.4 μM (UKE1) 3.94 μM (MDA-MB- 231) |
| 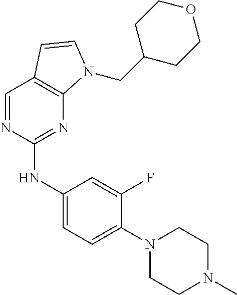 [SMILES: CN1CCN(c2ccc(Nc3ncc4ccn(CC5CCOCC5)c4n3)cc2F)CC1] | <250 nM | | |
| 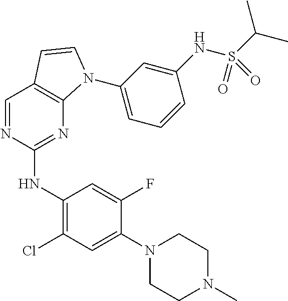 [SMILES: CC(C)S(=O)(=O)Nc1cccc(-n2ccc3cnc(Nc4cc(F)c(N5CCN(C)CC5)cc4Cl)nc32)c1] | <250 nM | | |
| 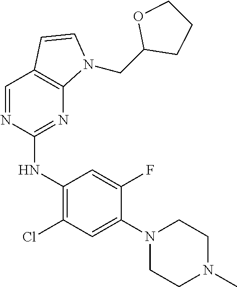 [SMILES: CN1CCN(c2cc(Cl)c(Nc3ncc4ccn(CC5CCCO5)c4n3)cc2F)CC1] | <250 nM | | |
| 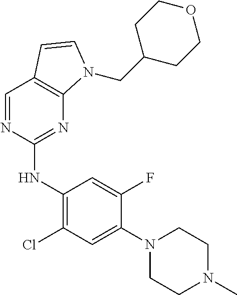 [SMILES: CN1CCN(c2cc(Cl)c(Nc3ncc4ccn(CC5CCOCC5)c4n3)cc2F)CC1] | <250 nM | 6.4 | |
| 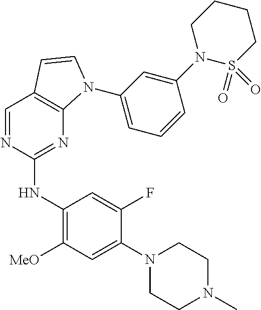 [SMILES: COc1cc(N2CCN(C)CC2)c(F)cc1Nc1ncc2ccn(-c3cccc(N4CCCCS4(=O)=O)c3)c2n1] | <250 nM | | 1.3 μM (UKE1) 3.40 μM (MDA-MB- 231) |
| 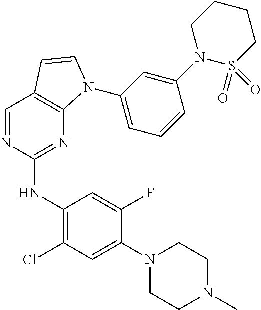 [SMILES: CN1CCN(c2cc(Cl)c(Nc3ncc4ccn(-c5cccc(N6CCCCS6(=O)=O)c5)c4n3)cc2F)CC1] | <250 nM | | |
| 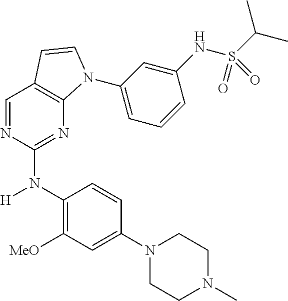 [SMILES: COc1cc(N2CCN(C)CC2)ccc1Nc1ncc2ccn(-c3cccc(NS(=O)(=O)C(C)C)c3)c2n1] | <250 nM | 7.6 | 2.7 μM (UKE1) 5.15 μM (MDA-MB- 231) |
| 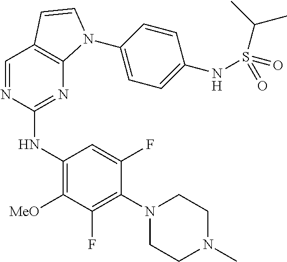 [SMILES: COc1c(Nc2ncc3ccn(-c4ccc(NS(=O)(=O)C(C)C)cc4)c3n2)cc(F)c(N2CCN(C)CC2)c1F] | <250 nM | | |
| 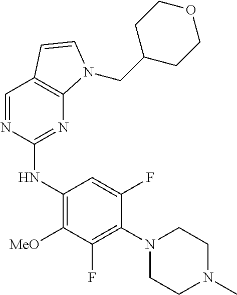 [SMILES: COc1c(Nc2ncc3ccn(CC4CCOCC4)c3n2)cc(F)c(N2CCN(C)CC2)c1F] | >1000 nM | | |
| 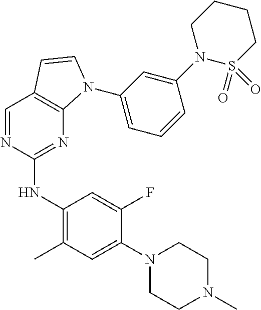 [SMILES: Cc1cc(N2CCN(C)CC2)c(F)cc1Nc1ncc2ccn(-c3cccc(N4CCCCS4(=O)=O)c3)c2n1] | ND | 6.2 | |
| 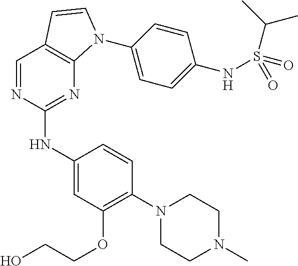 [SMILES: CC(C)S(=O)(=O)Nc1ccc(-n2ccc3cnc(Nc4ccc(N5CCN(C)CC5)c(OCCO)c4)nc32)cc1] | ND | 5.3 | |
| 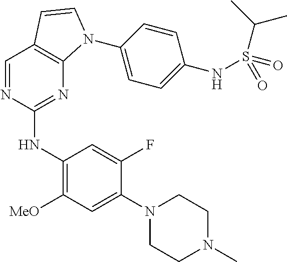 [SMILES: COc1cc(N2CCN(C)CC2)c(F)cc1Nc1ncc2ccn(-c3ccc(NS(=O)(=O)C(C)C)cc3)c2n1] | ND | 9.3 | |
| 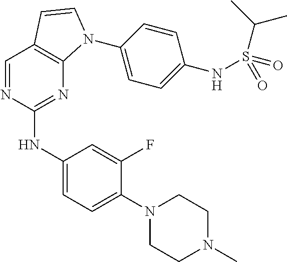 [SMILES: CC(C)S(=O)(=O)Nc1ccc(-n2ccc3cnc(Nc4ccc(N5CCN(C)CC5)c(F)c4)nc32)cc1] | ND | 6.1 | |
| 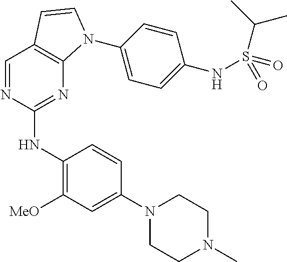 [SMILES: COc1cc(N2CCN(C)CC2)ccc1Nc1ncc2ccn(-c3ccc(NS(=O)(=O)C(C)C)cc3)c2n1] | ND | 6.5 | |
| 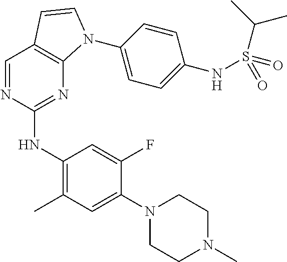 [SMILES: Cc1cc(N2CCN(C)CC2)c(F)cc1Nc1ncc2ccn(-c3ccc(NS(=O)(=O)C(C)C)cc3)c2n1] | ND | 7.3 | |
| 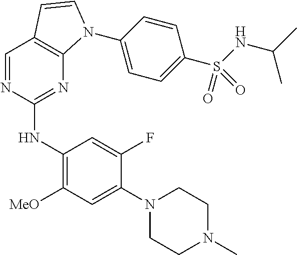 [SMILES: COc1cc(N2CCN(C)CC2)c(F)cc1Nc1ncc2ccn(-c3ccc(S(=O)(=O)NC(C)C)cc3)c2n1] | ND | 4.4 | |
| 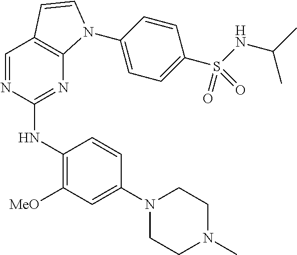 [SMILES: COc1cc(N2CCN(C)CC2)ccc1Nc1ncc2ccn(-c3ccc(S(=O)(=O)NC(C)C)cc3)c2n1] | ND | 8.1 | |
| 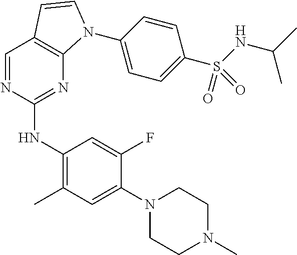 [SMILES: Cc1cc(N2CCN(C)CC2)c(F)cc1Nc1ncc2ccn(-c3ccc(S(=O)(=O)NC(C)C)cc3)c2n1] | ND | 7.7 | |
| 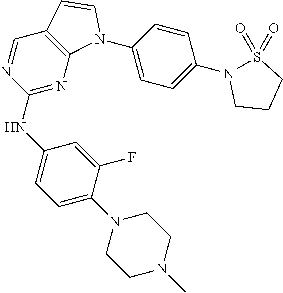 [SMILES: CN1CCN(c2ccc(Nc3ncc4ccn(-c5ccc(N6CCCS6(=O)=O)cc5)c4n3)cc2F)CC1] | ND | 5.6 | |

ND: not determined: DSF = Differential Scanning Fluorimetry, thermal shift

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula I 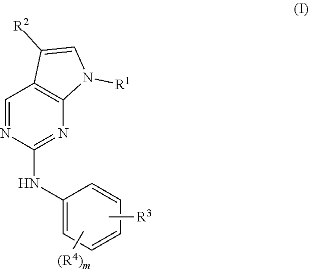
or a pharmaceutically acceptable salt thereof,
wherein:
R¹ is selected from —(C₀-C₃ alkyl)(3- to 8-membered monocyclic or bicyclic heterocyclyl) and —(C₀-C₃ alkyl)(6- to 10-membered monocyclic or bicyclic aryl), wherein R¹ may be optionally substituted with one or more groups selected from Z as allowed by valency;
R² is selected from hydrogen, halogen, and C₁-C₆ alkyl;
R³ is selected from —(C₀-C₃ alkyl)(3- to 8-membered monocyclic or bicyclic heterocyclyl) and —(C₀-C₃ alkyl)(5- to 10-membered moncyclic or bicyclic heteroaryl), wherein R³ may be optionally substituted with one or more groups selected from Z as allowed by valency;
m is 0, 1, 2, 3, or 4;
R⁴ is independently selected at each occurrence from hydrogen, halogen, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, each of which may be substituted one or more groups selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. (canceled)

3. The compound of claim 1, wherein R¹ is —(C₀-C₃ alkyl)(5- to 6-membered monocyclic or bicyclic heterocyclyl) optionally substituted with one or more Z groups.

4. The compound of claim 1, wherein R¹ is —(C₀-C₃ alkyl)(tetrahydrofuranyl or tetrohydropyranyl) optionally substituted with one or more Z groups.

5. The compound of claim 1, R¹ is selected from —CH₂(tetrahydrofuranyl) or —CH₂(tetrahydropyranyl) optionally substituted with one or more Z groups.

6. The compound of claim 1, wherein R¹ is selected from:

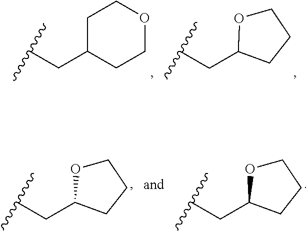

7. (canceled)

8. The compound of claim 1, wherein R¹ is —(C₀-C₃ alkyl)(phenyl) optionally substituted with one or more Z groups.

9. The compound of claim 1, wherein R¹ is selected from:

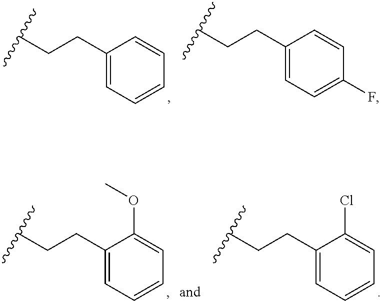

10. The compound of claim 1, wherein R¹ is phenyl optionally substituted with one or more Z groups.

11. The compound of claim 1, wherein R¹ is phenyl substituted with a group selected from —NHS(O)₂(C₁-C₆ alkyl) and 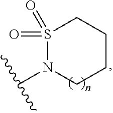
wherein n is 0 or 1.

12. The compound of claim 1, wherein R¹ is selected from:

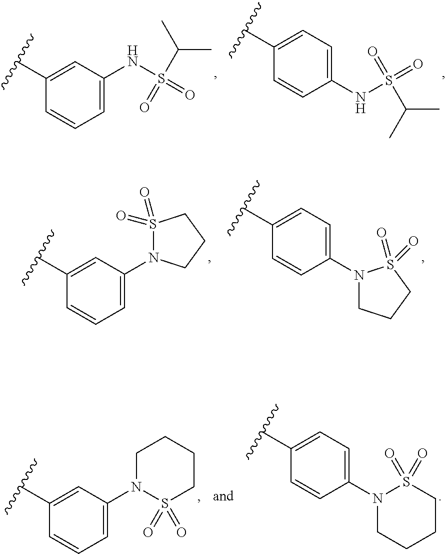

13. The compound of claim 1, wherein R² is selected from hydrogen, fluoro, and methyl.

14-16. (canceled)

17. The compound of claim 1, wherein 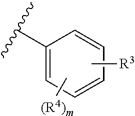
is selected from:

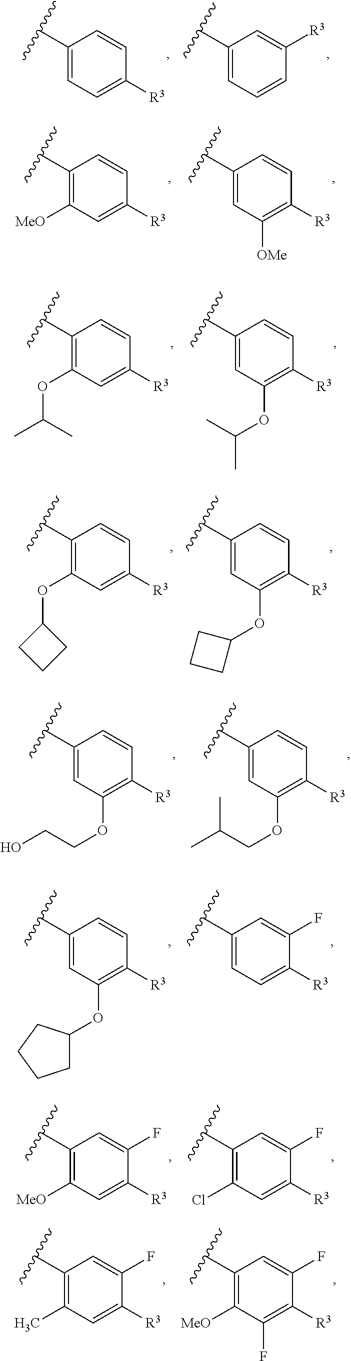

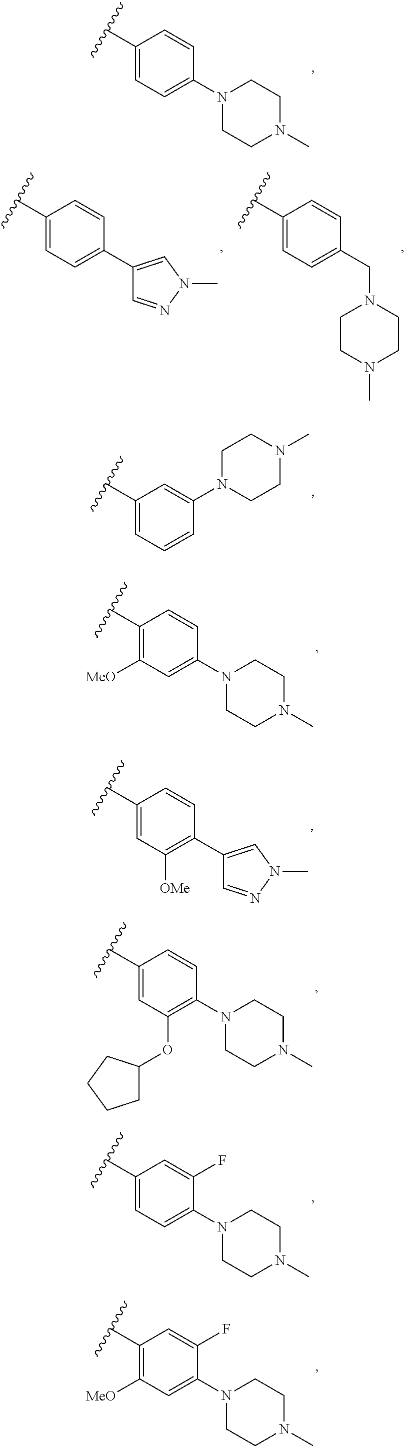

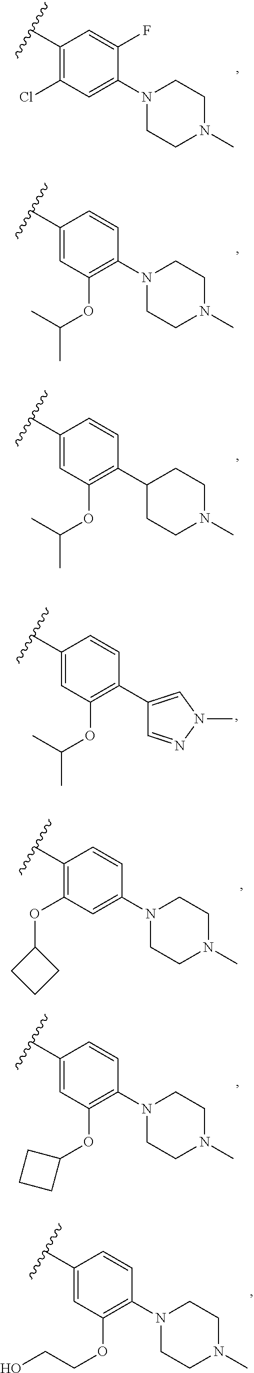

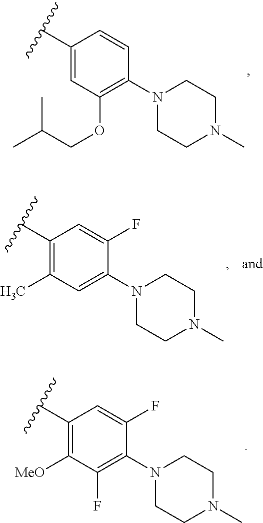 [SMILES: *c1ccc(N2CCN(C)CC2)c(OCC(C)C)c1 ; *c1cc(F)c(N2CCN(C)CC2)cc1C ; *c1cc(F)c(N2CCN(C)CC2)c(F)c1OC]

18. The compound of claim 1, wherein R³ is selected from:

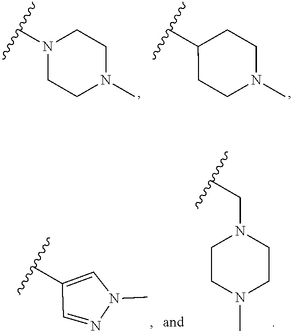

19. (canceled)

20. A compound selected from: 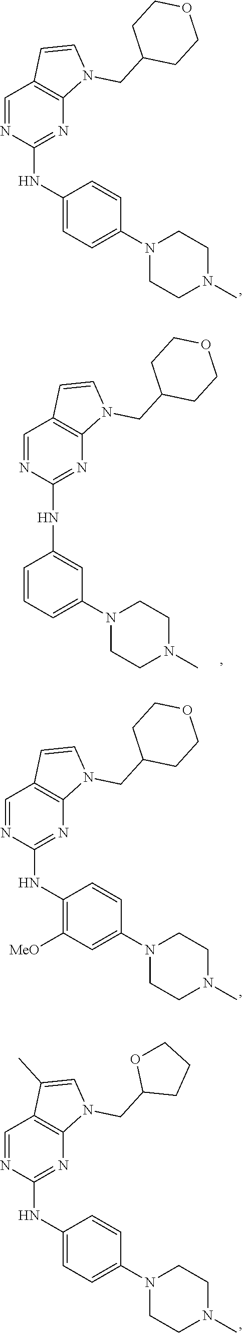 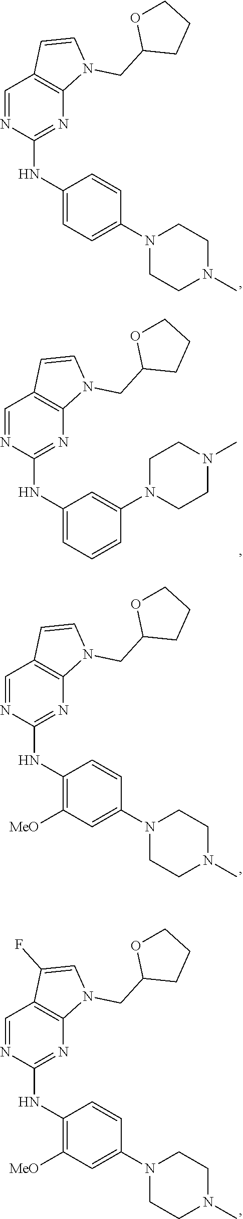 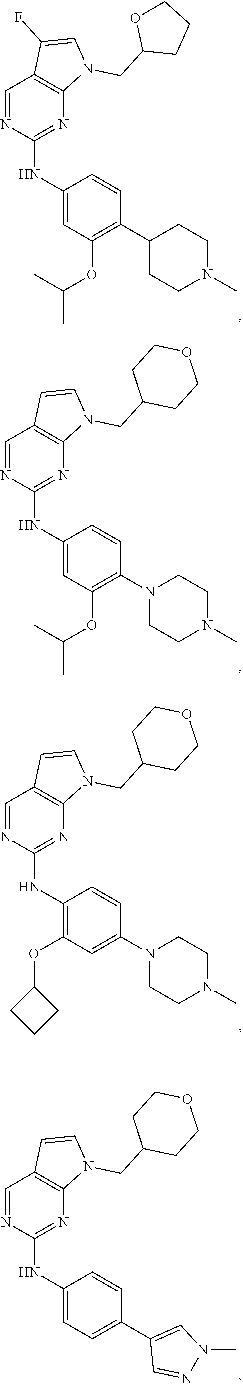 [SMILES: CC(C)Oc1cc(Nc2ncc3c(F)cn(CC4CCCO4)c3n2)ccc1C1CCN(C)CC1 ; CC(C)Oc1cc(Nc2ncc3ccn(CC4CCOCC4)c3n2)ccc1N1CCN(C)CC1 ; CN1CCN(c2ccc(Nc3ncc4ccn(CC5CCOCC5)c4n3)c(OC3CCC3)c2)CC1 ; Cn1cc(-c2ccc(Nc3ncc4ccn(CC5CCOCC5)c4n3)cc2)cn1] 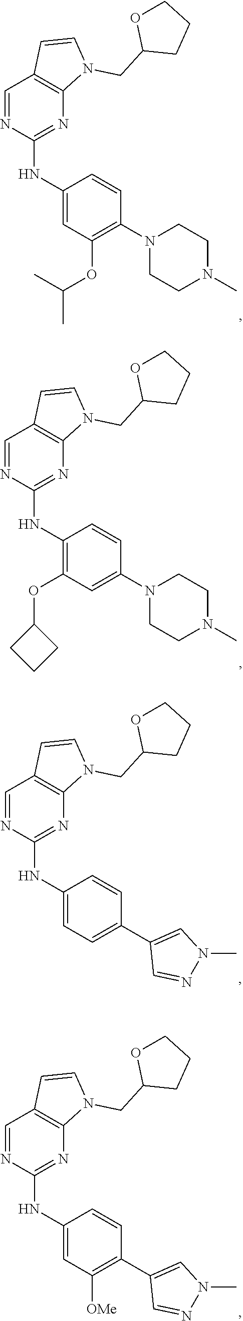 [SMILES: CC(C)Oc1cc(Nc2ncc3ccn(CC4CCCO4)c3n2)ccc1N1CCN(C)CC1 ; CN1CCN(c2ccc(Nc3ncc4ccn(CC5CCCO5)c4n3)c(OC3CCC3)c2)CC1 ; Cn1cc(-c2ccc(Nc3ncc4ccn(CC5CCCO5)c4n3)cc2)cn1 ; COc1cc(Nc2ncc3ccn(CC4CCCO4)c3n2)ccc1-c1cnn(C)c1] 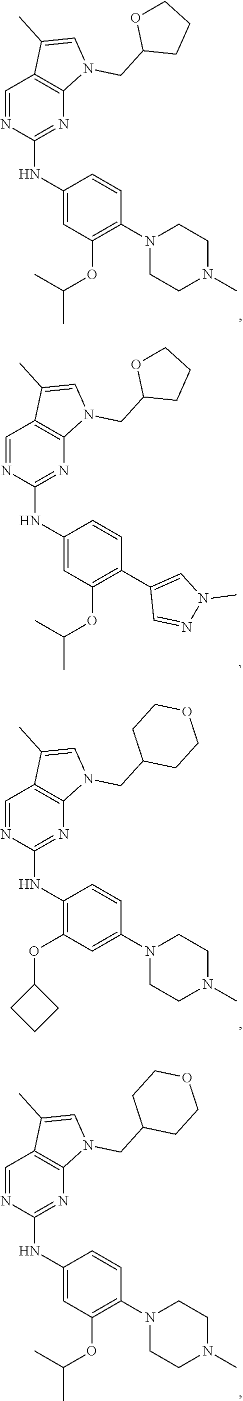 [SMILES: Cc1cn(CC2CCCO2)c2nc(Nc3ccc(N4CCN(C)CC4)c(OC(C)C)c3)ncc12 ; Cc1cn(CC2CCCO2)c2nc(Nc3ccc(-c4cnn(C)c4)c(OC(C)C)c3)ncc12 ; Cc1cn(CC2CCOCC2)c2nc(Nc3ccc(N4CCN(C)CC4)cc3OC3CCC3)ncc12 ; Cc1cn(CC2CCOCC2)c2nc(Nc3ccc(N4CCN(C)CC4)c(OC(C)C)c3)ncc12] 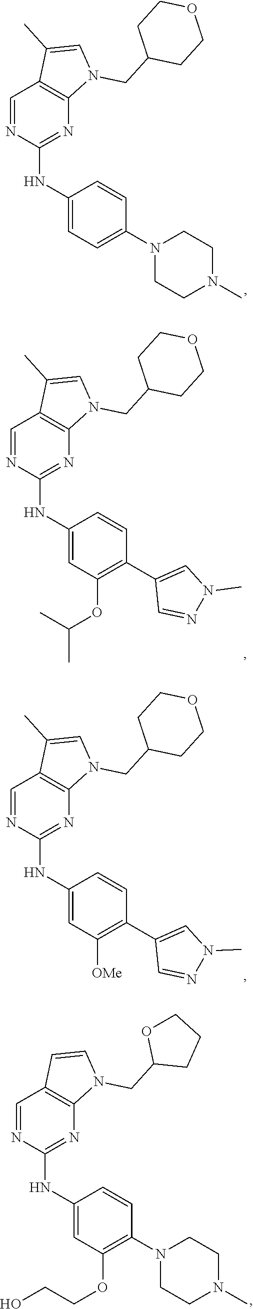 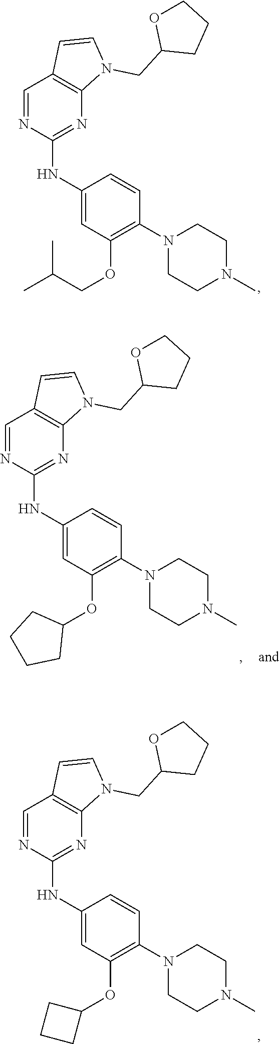 [SMILES: CC(C)COc1cc(Nc2ncc3ccn(CC4CCCO4)c3n2)ccc1N1CCN(C)CC1 ; CN1CCN(c2ccc(Nc3ncc4ccn(CC5CCCO5)c4n3)cc2OC2CCCC2)CC1 ; CN1CCN(c2ccc(Nc3ncc4ccn(CC5CCCO5)c4n3)cc2OC2CCC2)CC1]
or a pharmaceutically acceptable salt thereof.

21. (canceled)

22. A pharmaceutical composition comprising a compound of claim 1, or a pharmaceutically acceptable salt thereof, and a pharmaceutically acceptable carrier or excipient.

23. A method of treating a cancer in a subject in need thereof comprising administering to the subject a therapeutically effective amount of a compound of claim 1, or a pharmaceutically acceptable salt thereof.

24. The method of claim 23, further comprising an additional therapeutic agent.

25. The method of claim 24, wherein the additional therapeutic agent comprises an anti-cancer agent or an anti-inflammatory agent.

26. The method of claim 23, further comprising administering an effective amount of ionizing radiation to the subject.

27. A method of killing a tumor cell comprising contacting the tumor cell with an effective amount of a compound of claim 1, or a pharmaceutically acceptable salt thereof.
